(19) United States
(12) Patent Application Publication
(10) Pub. No.: US 2025/0101511 A1
(43) Pub. Date: Mar. 27, 2025

(54) OLIGO-MEDIATED DEPOSITION OF FUNCTIONAL MOLECULES FOR SPATIALLY RESOLVED GENOMICS, TRANSCRIPTOMICS, AND PROTEOMICS

(71) Applicant: UNIVERSITY OF WASHINGTON, Seattle, WA (US)

(72) Inventors: David SHECHNER, Seattle, WA (US); Brian BELIVEAU, Seattle, WA (US); Shreeram AKILESH, Seattle, WA (US); Sahar ATTAR, Seattle, WA (US); Conor CAMPLISSON, Seattle, WA (US); Evan KANIA, Seattle, WA (US); Ashley TSUE, Seattle, WA (US)

(21) Appl. No.: 18/728,937

(22) PCT Filed: Jan. 13, 2023
(86) PCT No.: PCT/US2023/060620

§ 371 Date: Jul. 15, 2024

Related U.S. Application Data
(60) Provisional application No. 63/300,125, filed on Jan. 17, 2022.

(51) Int. Cl. C12Q 1/6874; C12Q 1/6841; G01N 33/68

(52) U.S. Cl. CPC C12Q 1/6874; C12Q 1/6841; G01N 33/6848; G01N 2458/10

(57) ABSTRACT

Disclosed are methods and kits for detecting and visualizing biomolecules of interest, RNA molecules, and/or genomic loci, as well as the proteins, RNAs, or chromatin loci associated with the biomolecules of interest, RNA molecules, or genomic loci. The methods and kits can be used for elucidating molecular interactions in situ.

### CROSS REFERENCES

This application claim priority to U.S. Provisional Patent Application Ser. No. 63/300,125, filed Jan. 17, 2022, incorporated by reference here in its entirety.

### STATEMENT OF GOVERNMENT SUPPORT

This invention was made with government support under Grant Nos. R01 GM138799 and R35 GM137916 and U24 DK115255, awarded by the National Institutes of Health. The govemment has certain rights in the invention.

### BACKGROUND

Within the context of the cell, very little RNA is naked. Direct binding interactions with other biomolecules (proteins, RNAs, genomic loci) regulate all aspects of an RNA's lifecycle, including its biogenesis, localization, turnover, and its protein-coding or noncoding functions^{4,5}. Moreover, higher-order interactions between transcripts and their local microenvironment are critical for organizing subcellular architecture and compartmentalization. In humans, for example, RNAs are central determinants of chromatin folding⁸⁻¹⁰, and they nucleate and scaffold a host of biomolecular condensates that collectively control cellular metabolic, epigenetic, and stress-signaling pathways¹¹⁻¹³. Yet, most of these critical structures have eluded detailed molecular characterization, in part due to a lack of robust methods for elucidating RNA subcellular interactions at both local (Å-nm) and compartment-level (nm-μm) distances^{6,14}.

Current RNA interaction-discovery approaches using biotinylated antisense oligonucleotides to pull down a target RNA and its molecular partners from cell lysates come with several key limitations. First, eliminating the spurious capture of off-target RNAs can be challenging. Second, they can be plagued by artifactual interactions with abundant, nonspecific RNA-binding proteins, leading to false positives. Overcoming this experimental background often requires large input masses (˜10⁸ cells), especially for low-abundance RNAs. Finally, these techniques have difficulty capturing higher-order interactions that depend upon intact subcellular structure, and which may not survive a pulldown.

Applying proximity-biotinylation to RNAs also has significant limitations. Unlike proteins, transcripts cannot be genetically fused to the biotinylating enzyme, and hence established strategies seek to engineer artificial complexes between the enzyme and its target RNA. These transgenic approaches produce substantial pools of mislocalized or unbound biotinylating enzymes, resulting in background labeling that can blur or exceed the experimental signal. These methods also require complex cell engineering to simultaneously overexpress multiple components, often including the RNA itself.

### SUMMARY DISCLOSURE

In a first aspect, the disclosure provides a method for detecting one or more proteins, RNAs or genomic loci associated with a biomolecule of interest including contacting a biological sample with a first probe to produce a modified sample. The first probe has a first binding site capable of binding the biomolecule of interest, and one or a plurality of second binding sites each capable of binding with a detector oligonucleotide. The modified sample includes the first binding site of the first probe bound to the biomolecule of interest. The method for detecting one or more proteins associated with a biomolecule of interest further includes contacting the modified sample with one or more detector oligonucleotides to produce a complexed sample. Each detector oligonucleotide has a first binding site capable of binding to one or more of the second binding sites of the first probe, and an enzyme. The complexed sample includes the first binding site of the first probe bound to the biomolecule of interest; and the first binding site of one or more of the detector oligonucleotides bound with a second binding site of the first probe one or plurality of second binding sites. The method for detecting one or more proteins associated with a biomolecule of interest further includes contacting the complexed sample with a substrate capable of being converted to a reactive visible form to produce a labeled sample. The labeled sample includes the first binding site of the first probe bound to the biomolecule of interest, the first binding site of one or more of the detector oligonucleotides bound to a second binding site of the first probe one or plurality of second binding sites, and a covalent linkage between the substrate and the one or more proteins associated with the biomolecule of interest. The method for detecting one or more proteins associated with a biomolecule of interest further includes detecting the one or more proteins which are associated with to the biomolecule of interest.

In a second aspect, the disclosure provides a method for detecting one or more proteins associated with an RNA molecule or a genomic locus of interest including contacting a biological sample with a first oligonucleotide to produce a modified sample. The first oligonucleotide has a first binding site capable of binding the RNA molecule or a genomic locus of interest, and one or a plurality of second binding sites each capable of base pairing with a second oligonucleotide. The modified sample includes the first binding site of the first oligonucleotide base paired with the RNA molecule or a genomic locus of interest. The method for detecting one or more proteins associated with an RNA molecule or a genomic locus of interest further includes contacting the modified sample with one or more second oligonucleotide to produce a complexed sample. Each second oligonucleotide bas a first binding site capable of base pairing to the second binding site of the first oligonucleotide, and horseradish peroxidase (HRP). The complexed sample includes the first binding site of the first oligonucleotide base paired with the RNA molecule or a genomic locus of interest; and the first binding site of one or more of the second oligonucleotide base paired with a second binding site of the first oligonucleotide one or plurality of second binding sites. The method for detecting one or more proteins associated with an RNA molecule or a genomic locus of interest further includes contacting the complexed sample with biotin tyramide and H₂O₂ to produce a biotinylated sample. The biotinylated sample includes the first binding site of the first oligonucleotide base paired with the RNA molecule or a genomic locus of interest, the first binding site of one or more of the second oligonucleotide base paired with a second binding site of the first oligonucleotide one or plurality of second binding sites, and a covalent linkage between the biotin tyramide and the one or more proteins associated with the RNA molecule or a genomic locus of interest. The method for detecting one or more proteins associated with an RNA molecule or a genomic locus of interest further includes detecting the one or more proteins which are associated with the RNA molecule or genomic locus of interest.

In a third aspect, the disclosure provides a method for visualizing a biomolecule of interest including contacting a biological sample with a first probe to produce a modified biological sample. The first probe has a first binding site capable of binding to a biomolecule of interest, and a plurality of second binding sites, each capable of binding to a detector oligonucleotide. The modified biological sample includes the first binding site of the first probe bound to the biomolecule of interest. The method for visualizing a biomolecule of interest further includes contacting the modified biological sample with one or more detector oligonucleotides to produce a complexed sample. Each detector oligonucleotide has a first binding site capable of binding to the second binding site of the first probe, and an enzyme. The complexed sample includes the first binding site of the first probe bound to the biomolecule of interest and the first binding site of each detector oligonucleotide bound to a second binding site of the first probes plurality of second binding sites. The method for visualizing a biomolecule of interest further includes contacting the biological sample with one more visualizing agents and visualizing the biomolecule of interest. The one or more visualizing agents are activated by the enzyme.

In a fourth aspect, the disclosure provides a kit including a first probe having a first binding site that binds to biomolecule of interest, and a plurality of second binding sites, each second binding site capable of binding to a detector oligonucleotide. The kit further includes a detector oligonucleotide having a first binding site capable of binding to the second binding site of the first probe, and an enzyme.

In a fifth aspect, the disclosure provides a kit including a first oligonucleotide having a first binding site that binds to biomolecule of interest, and a plurality of second binding sites, each second binding site capable of base pairing with a second oligonucleotide. The kit further includes a second oligonucleotide having a first binding site capable of base pairing to the second binding site of the first oligonucleotide, and an enzyme.

### BRIEF DESCRIPTION OF FIGURES

FIG. 1 shows a scheme for O-MAP Design and Implementation. a. Overview of O-MAP. Specimens are chemically fixed, and pools of antisense DNA probes are hybridized to the target RNA. These probes recruit a common, horse-radish peroxidase (HRP)-conjugated secondary probe that catalyzes in situ proximity-biotinylation. b. O-MAP enables precise RNA-targeted proximity labeling, enabling interaction-discovery. In situ biotinylation imaged by neutravidin staining; NPM1 by immunofluorescence. Note nucleolar biotinylation in 47S O-MAP, nucleoplasmic biotinylation in 7SK O-MAP. c. Nucleolar-targeted APEX2 exhibits substantial off-target, nucleoplasmic labeling. d. O-MAP Probe Validation Assay. Primary probes are split into sub-pools that enable O-MAP and RNA-FISH to be performed simultaneously. Lack of co-localization suggests probe off-targeting. e. Probe Validation Assays on 47S-pre-rRNA and 7SK, in Hela cells, and Xist, in mouse Patski cells. f. Recovery of O-MAP-biotinylated proteins. Top: Streptavidin-HRP blot of whole cell lysates. Note ladder of biotinylated proteins from 47S O-MAP Bottom: Ponceau stain. All scale bars: 20 μm.

FIG. 2 shows O-MAP-MS for probing RNA-proximal proteomes. a. Strategy for characterizing the HeLa nucleolar proteome. Parallel O-MAP experiments targeting the 47S (nuclcolar), 7SK (nucleoplasmic) or using scrambled probes (background) were processed as indicated. Samples were quantified by TMTPro mass-spectrometry; each replicate (n=3) was labeled with a unique mass-tag. b-d. analysis of a single-shot 47S/7SK O-MAP-MS experiment, using 5.5×10⁶ cells per replicate. b. Recovery of known nucleolar proteins (left) and 7SK interactors (right). Two different probe sets, requiring different labeling times, were used in 7SK experiments; these were matched to scrambled-probe controls. c. Global enrichment of known nucleolar and nucleplasmic marker proteins, defined by the Human Protein Atlas (HPA). Histogram (top) plots the enrichment of proteins in each compartment (nucleolar, nucleoplasmic) independently, not the total combined proteome. d. Unbiased analysis of all significantly enriched (p_{adj}≥0.05; log₂ (fold change)≥2) proteins, called as tentative 47S-proximal and 7SK-proximal interactors. For each group, the ten highest ranked Gene Ontology (GO) terms, and Gene Set Enrichment Analysis of a top GO Biological Process (GOBP) term are shown. e-g. Higher coverage of the nucleolar proteome using an O-MAP in situ biotinylation time course. e. Approach. 47S O-MAP was performed in parallel for the indicated times, enriched as TMTPro-labeled, as above (n=3). A single 7SK probe set and time point was used for normalization. In situ biotinylation visualized by neutravidin staining. Scale bars: 20 μm. f. k-medoid clustering yields a clade of 313 high-confidence nucleolar proteins. Notable marker genes are indicated. g. nearly all (87%) members of the nucleolar medoid group corresponds to annotated or manually curated nucleolar proteins (FIG. 13) shows RNA-FISH validation of the 47S pre-rRNA-targeting probe set.

FIG. 3 shows O-MAP-Seq for probing RNA-proximal transcripts. a. In situ biotinylated proteins are used to enrich nearby transcripts, which are then quantified by RNA-sequencing. b-f. O-MAP-Seq characterization of the HeLa nucleolar transcriptome, b. Volcano plots of 47S O-MAP-Seq data, demonstrating enrichment of snoRNAs, IncRNAs, pseudogenes (left), and de-enrichment of mRNAs (right) (n=3). c. Summary of enriched and de-enriched RNA classes in the nucleolar transcriptome. d. Nucleolar transcripts expressed from protein-coding genes are predominantly noncoding variants; de-enriched transcripts are predominantly mRNAs. NSD: nonstop decay; NMD: nonsense-mediated decay. e. Nearly half of the nucleolar transcriptome is encoded from loci within nucleolar-associated chromatin loci (NADs; FIG. 4). Fisher's exact test. f. O-MAP-Seq identifies novel nucleolar-localized transcripts. Note co-localization between ENSG00000-286147.1 RNA-FISH and NPM1 Immunofluorescence (arrows; highlighted in zoom insets), quantified on right. g. Nucleolar transcripts are enriched in Transposable Element (TE) domains. Left: Z-scores of variance stabilizing transformed (VST) data, corresponding to major TE families. Right: volcano plot of individual TE classes in 47S O-MAP-Seq data. h-j. Characterizing transcripts near the inactive X-chromosome (Xi), in Patski cells. h. Volcano plots of Xist O-MAP-Seq data. Left: enrichment for X-linked transcripts that escape X-chromosome inactivation (XCI) and autosomal transcripts, including several chromatin-regulatory IncRNAs (indicated). Right: enrichment for several classes of X-linked transposable elements. i. Gm14636 is a novel XCI-escape gene.Left: Gm14636 enrichment by O-MAP-Seq. Middle: co-localization of Gm14636 RNA-FISH and Xist O-MAP Note penetrant mono- or bi-allelic expression from the Xa (black arrows) and/or Xi (white arrowheads), quantified on the right. j, Kcnq1ot1 localizes near the Xi. Panel arrangement parallels that of i.

FIG. 4 shows O-MAP-ChIP for probing RNA-proximal genomic loci. a. In situ biotinylated proteins are used to enrich nearby chromatin loci, which are then quantified by DNA-sequencing. b-d. O-MAP-ChIP characterization of Xist genomic interactions, in Patski cells. b. Xist O-MAP-ChIP predominantly labels the X-chromosome (right, and inset). Data for the entire mouse genome are shown; Xist genomic locus is noted below. c. Xist O-MAP-ChIP is specific to the inactive X-chromosome (Xi). Histogram of Allelic proportions for ChIP data, quantified using SNPs specific to the Xi and Xa⁷⁸. d. Putative interactions between autosomal loci and the Xi. The kcnq1ot1 locus—but not MALAT1 and NEAT1 loci—appear enriched in Xist O-MAP-ChIP data. L2FC: log₂(Fold change, Enriched/Input) e-i. O-MAP-ChIP characterization of Nucleolar Associated Domains (NADs). e. 47S-targeted O-MAP in Hela cells recapitulates the known human NAD architecture. Most of chromosome 8 is shown. f. Conservation of NAD architecture between HeLa and HT1080 cells. g. Parallelized analysis of NAD architecture across four Pancreatic Ductal Adenocarcinoma (PDA) cell lines. Upset Plot summarizing NAD conservation, or lack thereof, between lines. The total number of NADs in each line appears nearly invariant. h. O-MAP-ChIP identifies NADs that are differentially regulated between Classical and Basal PDA subtypes. Examples of constitutive (left) and Differential (right) NADs on Chromosome 14 are shown. i. ChromHMM⁸⁰ analysis reveals differential enrichment of chromatin signatures among HeLa and PDA cell line NADs.

FIG. 5 shows O-MAP is readily ported across specimen types. a. 47S-O-MAP in cultured mammalian cell lines. NPM1 immuno-fluorescence denotes nucleoli. All human-derived lines used the same probe set and hybridization conditions as HeLa cells (FIGS. 1-4); MEFs used an analogous mouse-targeting probe set. b. 47S O-MAP in human patient-derived pancreatic ductal adenocarcinoma (PDA) organoids. FBL immunofluorescence denotes nucleoli. c, 47S O-MAP in cryo-preserved mouse tissue slices. All scale bars: 20 μm.

FIG. 6 shows O-MAP is broadly applicable to different RNA targets. a. O-MAP Probe Validation Assay (FIG. 1d) applied to a compendium of target transcripts. Note conspicuous overlap between RNA-FISH and O-MAP signals, Images from HeLa (47S, 7SK, MALAT1, NEAT1), Patski (Xist, Kcnq1ot1, Firre), patient-derived fibroblast^{#} (WDR7), and U2OS cells ((CxG)n and (G4C2)n RNAs). Firre is expressed from a single-copy transgene; (CxG)n and (G4C2)n RNAs are artificial constructs under doxycycline-inducible expression^{#}; all other targets are endogenous transcripts. Insets show zoomed-in sections of the same images, to highlight signal overlap. b. O-MAP at nascent transcripts probes subnuclear neighborhoods. Left: Although mature Xist coats the entire inactive X-chromosome (Xi), nascent Xist transcripts uniquely denote the X-inactivation center (XCI). Right: O-MAP targeting Xist introns induces biotinylation at confined foci within the Xi “cloud.” All scale bars: 20 μm.

FIG. 7 shows RNA-FISH validation of the 47S pre-rRNA-targeting probe set. a. Schematic of the 47S pre-rRNA. During ribosome biogenesis, the 18S, 5.8S, and 28S domains are processed and incorporated into mature ribosomes, while the 5′- and 3′-External Transcribed Spacers (5′-ETS and 3′-ETS) and Internal Transcribed Spacers (ITS1 and ITS2) are cleaved from the precursor transcript and degraded within the nucleolus. The 47S pre-rRNA probe set used in the initial stages of O-MAP development target ITS1. b. validation of this probe set. HEK293T cells were transiently transfected with GFP-tagged NPM1, a nucleolar marker, and subjected to conventional RNA-FISH. Note conspicuous overlap between the NPM1˜GFP and ITS1 RNA-FISH signals. Scale bar: 20 μm.

FIG. 8 shows the alternate HRP-recruitment strategies tested. Overview and limitations of preliminary O-MAP designs. a. summary of RNA-targeted HRP-recruitment strategies tested. Design 1 uses biotinylated primary probes to recruit a streptavidin-HRP conjugate. Designs 2 and 3 use Digoxigenin (DIG)-labeled primary or secondary probes to recruit an HRP-conjugated anti-DIG antibody. Our final O-MAP design, which uses HRP-conjugated oligo probes, is shown for comparison (see also, FIG. 1a). The same anti-DIG antibody is used in designs 2 and 3; the same “universal landing pad” sequences are used in designs 3 and 4. b-e. limitations of Designs 1-3. In all cases biotin was visualized by staining with a fluorescent neutravidin conjugate. b. Design 1 was disfavored because in situ biotinylation cannot be unambiguously distinguished from biotinylated primary probes. HeLa cells over-expressing NPM1˜cGFP were probed using the same 47S-targeting probes as in (Supplementary FIG. 1) and the main text, appended on their 3′-termini with biotin. Note nucleolar biotin signal even in the absence of biotin-tyramide (bottom panels). We anticipated that this background signal would be especially problematic with low-abundance target RNAs. c. Design 2—analogous to HyPro™ (PMID: 35457249)—was sometimes capable of producing well-resolved nucleolar-targeted biotinylation. Hela cells are shown. This approach was eventually disfavored due to antibody irreproducibility issues described below, and because the high cost of DIG-labeled oligos would limit its use with low-abundance transcripts, which can require dozens to hundreds of probes. d. Design 3 overcomes the oligo cost issue but still suffers from antibody background binding and irreproducibility. HeLa cells were probed with the same 47S-targeting primary probe sets used in the main text (appended with the same “landing pad” modules), a DIG-labeled secondary oligo, and four different lots or vendors of commercial HRP-conjugated antibodies. In some cases, we observed well-resolved RNA-targeted biotinylation (panel i), though other lots from the same vendor exhibited off-target labeling (panel ii, arrows). Regents from other vendors exhibited varying degrees of spatial blurring (panel iii), or conspicuous off-target biotinylation that rivaled or exceeded the target signal (panel iv). d. Design 3 is particularly problematic with lower-abundance RNA targets. Patski cells were probed with the same Xist-targeting, landing-pad-extended probes as used in the main text, divided into two sub-pools. Anti-DIG-HRP was from Vendor 1. Note that all conditions—both probe sub-pools and the omit-primary negative control-induced substantial off-target biotinylation. Compare these results to (FIG. 1e). All scale bars, 20 μm.

FIG. 9 shows reproducibility of O-MAP labeling across primary probe sets. a. Targeting the 47S pre-rRNA. Probe sets targeting the 5′-ETS, ITS1, and ITS2 Transcribed Spacer domains (FIG. 7a) produced similar patterns of nucleolar in situ biotinylation. Probes targeting the 3′-ETS yielded no signal (data not shown). b. Targeting 7SK. Each set targets the entirety of the 7SK transcript, but were designed to have different hybridization parameters. Note that in situ biotinylation and exposure times differed between 7SK probe sets, as indicated (right). Biotin was visualized by staining with fluorescent neutravidin, in Hela cells. Scale bar: 20 μm.

FIG. 10 shows more O-MAP controls. O-MAP and negative control experiments were performed in HeLa cells, as indicated. Biotin was imaged using a fluorescent neutravidin conjugate; NPM1 via immunofluorescence. Note that omitting any component of the O-MAP pipeline ablated biotinylation signal. The 47S-O-MAP, 7SK-O-MAP, omit primary and scrambled primary conditions (left four columns) are the same images presented in (FIG. 1b). In the “omit-H₂O₂” condition (far right, marked *), cells were pre-quenched with sodium azide and ascorbic acid prior to the addition of biotin-phenol and H₂O₂. Simply removing H₂O₂ from the O-MAP protocol still resulted in targeted in situ biotinylation (i.e. nucleolar labeling using 47S probes), presumably due to photoactivation of HRP. Scale bars, 20 μm.

FIG. 11 shows enrichment of biotinylated proteins for O-MAP-MS. HeLa cells were O-MAP labeled using 47S-targeting, 7SK-targeting, or scrambled probes, as indicated. For 7SK, probe set 1 was used. Cells were lysed by boiling in SDS (see methods), enriched by streptavidin pulldown, and released from the beads by again boiling in SDS. 10 μg samples of the starting lysates (“input”) or eluted proteins (“Elute”) were separated on 10% SDS PAGE gels. a. SYPRO Ruby™-stained gel. The blurriness of bands in the input samples is a consequence of formaldehyde crosslinking. b. Streptavidin˜HRP blotting of the same gel, to visualize biotinylated proteins. Note the ladder of biotinylated products in both 47S- and 7SK-conditions, and the enrichment of these proteins in the eluted samples. Longer exposure (right) reveals a weak background of biotinylated material in the Scrambled probe control.

FIG. 12 shows Reproducibility of O-MAP-MS. a. Average coefficients of Variation (CV) between biological replicates (n=3). Conventionally, CV's below 0.2 are considered acceptable. b. Correlation matrix between all samples. Note high positive correlations between biological replicates, highlighting O-MAP's reproducibility, and negative correlations between different sample types (e.g. 47S vs 7SK 1 minute), highlighting the unique composition of the proteome captured for each target. c. Heatmap of all proteomic data, clustered by unsupervised hierarchical clustering, showing defined clades of 47S-proximal and 7SK-proximal proteomes. Note that the 7SK 1-minute labeling condition used probe set 1; the 10-minute condition used probe set 2. d. Scatter plots comparing scaled protein abundances for individual biological replicates for the Scramble negative control (10 minutes' labeling time, left), 7SK probe set two (center), and 47S (right). Data are plotted on log2 scale.

FIG. 13 shows further validation of the one-shot 47S/7SK O-MAP-MS dataset. a. Enrichment profiles (as in FIG. 2b) of core components of the 7SK particle (left) and of recently discovered 7SK-interactors, including members of the BAF complex (right). n=3. Core interactors LARP7 and HEXIM2 were not detectable above noise. b. Differential enrichment of the nuclear proteome, defined by the Human Protein Atlas (HPA) as being strictly nucleolar, strictly nucleoplasmic or bilocalized between the two compartments. Fold changes calculated using the same 47S-targeting probe set, relative to either 7SK-targeting probe pool 1 (left), or 2 (right). In both cases, note the strong enrichment of nucleolar proteins, intermediate enrichment of bilocalized proteins, and de-enrichment of nucleoplasmic proteins. Note that the two 7SK probe sets gave nearly identical results. This is further supported by: c. the total number of proteins from each class that were observed with both probe sets, and d. correlation between average protein abundances observed using each probe set.

FIG. 14 shows probing the nucleolar proteome with 47S O-MAP-MS. a. Receiving-Operator Characteristic (ROC) analysis of the 47S vs 7SK O-MAP-MS experiment. True Positive and False Positive proteins were defined using lists of exclusively nucleolar and exclusively nucleoplasmic proteins, respectively, as reported by the Human Protein Atlas (HPA). An Area Under the Curve (AUC) of nearly 1.0 suggests strong and highly sensitive selectivity for nucleolar proteins over the nucleoplasmic proteome. b. These data were used to derive an optimal Log2 (fold change, 47S/7SK) cutoff value of 0.523, and to define a putative list of 258 O-MAP core nucleolar proteins, as described in (FIG. 2). c-e, parallel analysis using (47S/Scramble controls), instead of (47S/7SK). c. Volcano plot and histograms of showing the enrichment of HPA-nucleolar, HPA-Nucleoplasmic, and HPA-bilocalized proteins, using the same protein marker reference lists as in (a-b), and (FIG. 2c,d). d. ROC analysis of the (47S/Scramble) data demonstrates slightly lower sensitivity than that of the (47S/7SK) analysis, though still exceptionally. e. As in (b), these data were used to determine an optimal Log2 (fold change, 47S/Scramble) cutoff value of 2.201, defining a putative cohort of 286 O-MAP core nucleolar proteins. f. the putative nucleolar proteomes derived from the (47S/7SK) and (47S/Scramble) ROC analyses show considerable overlap (66-73%). Outliers were used for Gene Ontology (GO)-term analysis. Factors uniquely captured by the (47S/7SK) analysis were highly enriched for ribosome biogenesis factors, while those unique to the (47S/Scramble) analysis were enriched for nucleoplasmic functions. This suggests that the (47S/7SK) comparison more precisely captures the nucleolar protcome.

FIG. 15 shows probing the 7SK-proximal proteome with O-MAP-MS. a. Identifying the optimal basis of comparison for probing the 7SK-proximal compartment: (7SK/47S) or (7SK/Scramble). For each comparison, volcano plots demonstrate the enrichment of the nucleolar-and nucleoplasmic-proteomes, as derived from O-MAP analysis (FIG. 2 and FIG. 14), and of known Nuclear Speckle proteins, markers of the 7SK-proximal compartment. Box-whisker plots (above) summarize the distribution of each protein group (significance testing: two-tailed, heteroscedastic Student's t-tests; n.s: not significant, * p=0.05; **** p<1×10−5). In each comparison, speckle proteins are significantly enriched relative to the nucleolar proteome, but in the (7SK/47S) comparison (left) these proteins are indistinguishable from the broader nucleoplasmic proteome (p=0.14). In contrast, in the (7SK/Scramble) comparison (right) Speckle proteins are significantly enriched relative to the nucleoplasmic outgroup (p=5×10−10). This suggests that (7SK/Scramble) is more selective for 7SK-proximal interactors over the broader nucleoplasmic proteome. This is corroborated by Receiver-Operating Characteristic (ROC) analysis, as described below, which was used to define the “7SK-proximal” cutoff in the righthand panel. b. ROC analysis of (7SK/47S), using speckle proteins as true positives and O-MAP-nucleolar proteins as false positives. An area under the curve (AUC) of approaching 0.5 suggests a nearly complete absence of signal. c. ROC analysis of (7SK/Scramble), using the same True Positive and False Positive lists as in (b), suggests a strong and specific separation between the protein populations, and an optimal Log2 (fold change) cutoff of ≥1.794. d. A nearly identical result is obtained when using O-MAP-nucleolar proteins as False Positives. These cutoffs define a cohort of 510 putative 7SK-proximal proteins. e. Gene Ontology (GO)-term analysis on this cohort is highly enriched for biological processes involved in pre-mRNA biogenesis.

FIG. 16 shows representative highly ranked Gene Set Enrichment Analysis (GSEA) results. a. GSEA results comparing 47S and 7SK O-MAP-MS. The same ranked gene list was used for all queries. Top Gene Ontology Biological Process (GOBP) and Cellular Component (GOCC) terms are shown. For all, left-ranking genes were more highly enriched from 47S O-MAP than 7SK O-MAP; those ranked toward the right were more enriched from 7SK O-MAP. b. GSEA results comparing 7SK O-MAP--MS to Scrambled controls. The same gene list was used for all queries. Leftmost genes were 7SK-enriched; rightmost were Scramble-enriched.

FIG. 17 shows k-medoid clustering. a. Determining the optimal number of clusters. Ultimately, 12 clusters were chosen, given the dip in experimental signal (arrowhead). b. Principle component analysis (PCA) of the twelve k-medoid clusters.

FIG. 18 coverage of the nucleolar proteome during the 47S O-MAP labeling time course. a. Volcano plots for each 47S O-MAP labeling point, calculated relative to the 7SK/10-minute label condition. Enrichment of nucleolar proteins derived from our k-medoid analysis (FIG. 2e-g; FIG. 16). b. Table summarizing the recovery of the nucleolar proteome (defined either by our first-pass analysis, FIGS. 2b-d, or k-medoid analysis), at each labeling time point. Note that coverage appears to plateau at 10 minutes.

FIG. 19 shows 47S O-MAP-Seq enriches known and novel nucleolar transcripts. a. the 47S pre-rRNA. Note prominent enrichment for the 5′-ETS, IT1, and ITS2 “transcribed spacer” domains. Sequences corresponding to the mature 18S, 5.8S, and 28S rRNAs are removed during sequencing library preparation. Reads are aligned to a custom genome assembly containing a single copy of the rDNA consensus sequence (courtesy of T. Moss, U. Laval) annotated as a unique chromosome. b. the U3 noncoding RNA, which directs key cleavage events during ribosomal biogenesis, c-d. exemplar Box C/D (c) and H/ACA (d) small nucleolar RNAs (snoRNAs). SnoRNAs are often expressed within the introns of protein-coding genes (gray). e. RNase MRP (enriched) upstream of the CCDC107 gene (not enriched). f. IncRNA SLERT, which is processed from the two H/ACA snoRNAs embedded in the TBRG4 gene. g. Example of a novel nucleolar transcript—a processed pseudogene—discovered by O-MAP-Seq

FIG. 20 shows 47S O-MAP-Seq and HyPro-Seq enrich common transcripts. Volcano plot of 47S O-MAP-Seq data, merging both coding and noncoding genes (FIG. 3b). Transcripts that were reportedly enriched by 47S HyPro-Seq (PMID: 34741808) are indicated. Note that differences in transcript annotation complicated systematic comparison to HyPro-Seq data. In all, only 53 of the significantly enriched transcripts reported by HyPro-Seq were detected in our data, all of which were also enriched by 47S O-MAP-Seq.

FIG. 21 shows Xist O-MAP-Seq enriches nascent transcripts of XCI-escape genes. a. Enrichment of the Xist gene itself. Note different scales for input RNA and O-MAP-Seq tracks. The lack of intronic reads suggests that O-MAP-Seq has predominantly targeted and captured the mature Xist transcript. The absence of reads mapping to the antisense noncoding RNA TsiX, a IneRNA that is monoallelically expressed from the Xa (gray), confirms that O-MAP-Seq is precisely labeling the Xi. b. Enriched XCI-escape genes appear to be nascent transcripts. In all cases, read densities for both Input RNA (gray) and O-MAP-Seq (black) are shown, using matched scales. Note prominent intronic read density for all XCI-escape genes (Shroom4, Pbdc1, Magee1, Mid1, Erdr1). Transcript structures denoting the most prominent isoforms are displayed; other nearby genes not known to escape XCI are denoted in gray. c. Xist O-MAP-Seq does not appear to preferentially capture nascent transcripts of non-XCI-escape genes. Two examples (Tspan6 and Rps4x) are shown, neither of which was enriched by Xist O-MAP-Seq. Note the absence of prominent intronic read density, suggesting that the intronic signatures observed in (b) are not general artifacts of the O-MAP-Seq pipeline.

FIG. 22 shows genomic maps of HeLa Nucleolar-Associated Domains (NADs). Data are Log₂(O-MAP-ChIP/Input). NADs were called by merging peaks from EDD and EPIC2 (black blocks). The same pipeline was also applied to HT1080 NADs, probed by fractionation-sequencing (Fraq-Seq; PMID 20826608). Although all replicates are shown, high noise/low information in Fraq-Seq replicate 2 resulted in EDD failure after 10,000 Monte Carlo simulations; only replicate 1 was used for NAD calls (orange blocks). The mitochondrial genome exhibits a complete absence of nucleolar interactions, as expected. Note that HeLa (O-MAP-ChIP) and HT1080 (Fraq-Seq) cells were derived from patients of different genders, which may explain the divergence in NAD architecture on the X-chromosome.

FIG. 23 shows 7SK O-MAP in cultured Pancreatic Ductal Adenocarcinoma (PDA) cell lines. O-MAP was visualized using a fluorescent neutravidin conjugate; NPMI by immunofluorescence, as in (FIG. 5). Scale bars: 20 μm

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R.I. Freshney. 1987. Liss, Inc. New York, NY), Gene Transfer and Expression Protocols, pp. 109-128, ed. E.J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Additionally, the words “herein,” “above,” and “below” and words of similar import, when used in this application, shall refer to this application as a whole and not to any particular portions of the application.

In a first aspect, the disclosure provides a method for detecting one or more proteins, RNAs or genomic loci associated with a biomolecule of interest including contacting a biological sample with a first probe to produce a modified sample. The first probe has a first binding site capable of binding the biomolecule of interest, and one or a plurality of second binding sites each capable of binding with a detector oligonucleotide. The modified sample includes the first binding site of the first probe bound to the biomolecule of interest. The method for detecting one or more proteins associated with a biomolecule of interest further includes contacting the modified sample with one or more detector oligonucleotides to produce a complexed sample. Each detector oligonucleotide has a first binding site capable of binding to one or more of the second binding sites of the first probe, and an enzyme. The complexed sample includes the first binding site of the first probe bound to the biomolecule of interest; and the first binding site of one or more of the detector oligonucleotides bound with a second binding site of the first probe one or plurality of second binding sites. The method for detecting one or more proteins associated with a biomolecule of interest further includes contacting the complexed sample with a substrate capable of being converted to a reactive visible form to produce a labeled sample. The labeled sample includes the first binding site of the first probe bound to the biomolecule of interest, the first binding site of one or more of the detector oligonucleotides bound to a second binding site of the first probe one or plurality of second binding sites, and a covalent linkage between the substrate and the one or more proteins associated with the biomolecule of interest. The method for detecting one or more proteins associated with a biomolecule of interest further includes detecting the one or more proteins which are associated with to the biomolecule of interest.

As used herein the biomolecule of interest can include, but is not limited to RNA, DNA, or a protein. In one non-limiting embodiment, the biomolecule of interest comprises a genomic locus.

As used herein the biological sample can be any kind sample suitable for use according to the methods, including but not limited to cells, organoids, and tissue sections. In non-limiting embodiments, the cells can be from cell cultures and the tissue sections can be isolated from whole organisms. In other non-limiting embodiments, the biological sample can be whole model organisms, including nematode worms, flies, and bacteria. The biological sample can be a fixed sample. In one non-limiting embodiment, the biological sample is a mammalian cell sample. In one non-limiting embodiment, the biological sample is a human biological sample.

As used herein the first probe can be any type of probe suitable for use according to the methods of the invention. These include, but are limited to, an oligonucleotide, an antibody, an aptamer, and/or a lectin.

Furthermore, the first probe can comprise a probe conjugated to an oligonucleotide. As used herein a “probe conjugated to an oligonucleotide” can include any macromolecule-specific probe, chemical, drug, or moiety which can be conjugated to an oligonucleotide. This includes, but is not limited to lectin or phalloidin.

According to the methods of the invention contacting a biological sample with a first probe produces a “modified sample.”

According to the methods of the invention the first probe comprises a first binding site capable of binding the biomolecule of interest, and one or a plurality of second binding sites each capable of binding with a detector oligonucleotide.

In various embodiments, the first probe comprises an oligonucleotide probe, wherein the one or a plurality of second binding sites on the first probe are each capable of base pairing with the detector oligonucleotide. As used herein the plurality of second binding sites can include any number of second binding sites suitable for use according the methods of the invention. On various non-limiting embodiments, the plurality of second binding sites comprises 2, 3, 4 or, 5 binding sites.

In one non-limiting embodiment, the plurality of binding sites on the first probe (oligo) can be produced using Primer Exchange reaction (PER) or signal amplification by enzymatic reaction (SABER). In other embodiments, the plurality of binding sites can be produced using rolling circle amplification, chemical ligation of DNA monomers, and/or any other suitable amplification method. In another non-limiting embodiment, the plurality of binding sites on the first probe (oligo) can be produced using PER, and SABER can be performed on the first probe after it binds to the biomolecule of interest.

As used herein “Primer Exchange reaction (PER)” is a method for non-templated synthesis of single stranded DNA molecules. PER can synthesize long concatemers containing many reiterated copies of the same short DNA sequence.

As used herein “signal amplification by enzymatic reaction (SABER)” are methods for highly multiplexed in situ signal amplification via hairpin-mediated concatemerization.

In one non-limiting embodiment, signal amplification by enzymatic reaction (SABER) can comprise any suitable combination of steps as disclosed in US Publication 2020/0362398, which is incorporated by reference herein.

In various embodiments the SABER methods typically includes 4 steps. The first step includes combining a sample containing a plurality of nucleic acid targets with a plurality of probe strands, each probe strand having (i) an unpaired 5′ target domain complementary to one of the nucleic acid targets and (ii) an unpaired 3′ primer domain, and producing a first reaction mixture comprising molecular targets bound to probe strands; and then the second step includes combining the first reaction mixture produced in step 1 with dNTPs, strand-displacing polymerase, and a plurality of catalytic molecules, each catalytic molecule having, 5′ to 3′, a first domain, a second domain, and a third domain wherein the first domain is bound to the second domain, and the third domain is an unpaired 3′ toehold domain complementary to the unpaired 3′ primer domain of one of the probe strands, and producing a second reaction mixture comprising nucleic acid concatemers bound to molecular targets. The third step includes combining the second reaction mixture produced in step 2 with a plurality of signal strands, each signal strand linked to a different detectable molecule and comprising a domain complementary to the unpaired 3′ primer domain of one of the probe strands, and producing concatemers labeled by a plurality of signal strands; and the fourth step optionally includes imaging the labeled concatemers.

In another embodiment the SABER methods can include a 4 step method where the first step includes combining a plurality of probe strands with dNTPs, strand-displacing polymerase, and a plurality of catalytic molecules. In this step each probe strand has (i) an unpaired 5′ target domain complementary to a nucleic acid target of a plurality of nucleic acid targets and (ii) an unpaired 3′ primer domain, and wherein each catalytic molecule comprises, 5′ to 3′, a first domain, a second domain, and a third domain wherein the first domain is bound to the second domain, and the third domain is an unpaired 3′ toehold domain complementary to the unpaired 3′ primer domain of one of the probe strands, and producing a first reaction mixture comprising nucleic acid concatemers bound to probe strands. Step 2 includes combining the first reaction mixture produced in step 1 with a sample containing the plurality of nucleic acid targets and producing a second reaction mixture comprising nucleic acid concatemers bound to molecular targets. Step 3 includes combining the second reaction mixture produced in step 2 with a plurality of signal strands, wherein each signal strand is linked to a different detectable molecule and comprises a domain complementary to the unpaired 3′ primer domain of one of the probe strands, and producing concatemers labeled by a plurality of signal strands. Finally step 4 optionally further includes imaging the labeled concatemers.

According to the methods of the invention the “modified sample” is then contacted with one or more detector oligonucleotides to produce a complexed sample, wherein each detector oligonucleotide comprises a first binding site capable of binding to one or more of the second binding sites of the first probe, and an enzyme. In another non-limiting embodiment, each detector oligonucleotide comprises a first binding site capable of binding to one or more of the second binding sites of the first probe, and another detector oligonucleotide.

As used herein “detector oligonucleotide” is any oligonucleotide for use suitable for use according to the methods of the invention, including but not limited to DNA, RNA, PNA, LNA, or morpholinos. In various non-limiting embodiments, the detector oligonucleotide can be conjugated to an enzyme, Digoxigenin (DIG), or a fluorophore. In other, non-limiting embodiments the detector oligonucleotide is capable of binding to an enzyme, Digoxigenin (DIG), or a fluorophore.

As used herein an enzyme can be any suitable enzyme for use according to the methods of the invention. In various, non-limiting embodiments, the enzyme comprises horseradish peroxidase (HRP), ascorbate peroxidase (APEX), HRP-streptavidin, alkaline phosphatase, or microbial transglutaminase.

According to the methods of the invention the “complexed sample” comprises the first binding site of the first probe bound to the biomolecule of interest; and the first binding site of one or more of the detector oligonucleotides bound with a second binding site of the first probe one or plurality of second binding sites.

According to the methods of the invention the “complexed sample” is then contacted with a substrate capable of being converted to a reactive visible form to produce a “labeled sample.”

As used herein a “substrate” can be any substrate suitable for use according to the methods of the invention. Suitable substrates include, but are not limited to a tyramide compound or biotin aniline, or salts thereof. Non-limiting examples of a tyramide compound include biotin tyramide, cyanine tyramide, alkyne tyramide, or a fluorescent tyramide, or salts thereof.

As used herein a “labeled sample” comprises the first binding site of the first probe bound to the biomolecule of interest, the first binding site of one or more of the detector oligonucleotides bound to a second binding site of the first probe one or plurality of second binding sites, and a covalent linkage between the substrate and the one or more proteins associated with the biomolecule of interest.

As used herein the covalent linkage is between the substrate capable of being converted to a reactive visible form and the one or more proteins. In various non-limiting embodiments, the second probe comprises ascorbate peroxidase (APEX) or HRP as the enzyme and a tyramide compound. In this emodiment the tyramide compound is biotin-tyramide. In this embodiment, the HRP catalyzes the covalent addition of biotin to the nearby one or more proteins which are associated with the biomocule of interest.

According to the methods of the invention the one or more proteins, RNAs or genomic loci which are associated with the biomolecule of interest in the labeled sample are then detected.

As used herein “detecting” or “detection” can be accomplished using any suitable methods, including, but not limited to in situ hybridization, in situ sequencing, immunohistochemistry, fluorescence in situ hybridization (FISH), RNA-FISH, or catalyzed reporter deposition (CARD)-FISH, tyramide signal amplification (TSA), streptavidin pulldown, affinity-capture, western-blotting, northern-blotting, southern-blotting, quantitative PCR, quantiative RT-PCR, mass-spectrometry, or RNA-seqeunceing or DNA-Sequencing. RNA-or DNA-Sequencing can include Sanger sequencing, illumina-based sequencing, NanoPore, PacBio, Ion Torrent, or NanoString sequencing. In various embodiments detecting can comprise visualizing the one or more proteins or the biomolecule of interest.

In various non-limiting embodiments, detecting can optionally further include lysing the labeled sample prior to detection in order to produce a lysed labeled sample. Lysing can be accomplished using any suitable method, including but not limited to chemical lysis, temperature lysis, osmotic lysis, enzymatic lysis, organic extraction, utlrasonic homogenization, mechanical homogenization, or lysis by French Press. Non-limiting examples of chemical lysis include RIPA buffer, cell lysis buffer (KCl and NP40 in Tris-HCl), and nuclear lysis buffer (NP40, SDS, Na Deoxycholate in Tris-HCl).

In various other non-limiting embodiments the methods can optionally further include lysing the complexed sample prior to contacting the complexed sample with a substrate to produce a labeled sample.

In a further non-limiting embodiment the method can include multiple detection steps using different detection methods. In various examples of this embodiment, the one or more proteins, RNAs, or genomic loci which are associated with the biomolecule of interest in the labeled sample is detected using a first detection method and then the one or more proteins or biomolecules of interest in the labeled sample are separated from each other. Then a second detection method is used. In one non-limiting example of this embodiment the one or more proteins, RNAs, or genomic loci which are associated with the biomolecule of interest in the labeled sample is first detected using fluorescence in situ hybridization. The one or more proteins or biomolecule of interest in the labeled sample are then separated from each other using lysis buffer and the one or more proteins or biomolecule of interest are detected a second time using mass-spectrometry, RNA-seqeunceing, or DNA-Sequencing.

In a second aspect, the disclosure provides a method for detecting one or more proteins associated with an RNA molecule or a genomic locus of interest including contacting a biological sample with a first oligonucleotide to produce a modified sample. The first oligonucleotide has a first binding site capable of binding the RNA molecule or a genomic locus of interest, and one or a plurality of second binding sites each capable of base pairing with a second oligonucleotide. The modified sample includes the first binding site of the first oligonucleotide base paired with the RNA molecule or a genomic locus of interest. The method for detecting one or more proteins associated with an RNA molecule or a genomic locus of interest further includes contacting the modified sample with one or more second oligonucleotide to produce a complexed sample. Each second oligonucleotide bas a first binding site capable of base pairing to the second binding site of the first oligonucleotide, and horseradish peroxidase (HRP). The complexed sample includes the first binding site of the first oligonucleotide base paired with the RNA molecule or a genomic locus of interest; and the first binding site of one or more of the second oligonucleotide base paired with a second binding site of the first oligonucleotide one or plurality of second binding sites. The method for detecting one or more proteins associated with an RNA molecule or a genomic locus of interest further includes contacting the complexed sample with biotin tyramide and H₂O₂ to produce a biotinylated sample. The biotinylated sample includes the first binding site of the first oligonucleotide base paired with the RNA molecule or a genomic locus of interest, the first binding site of one or more of the second oligonucleotide base paired with a second binding site of the first oligonucleotide one or plurality of second binding sites, and a covalent linkage between the biotin tyramide and the one or more proteins associated with the RNA molecule or a genomic locus of interest. The method for detecting one or more proteins associated with an RNA molecule or a genomic locus of interest further includes detecting the one or more proteins which are associated with the RNA molecule or a genomic loci of interest.

In a third aspect, the disclosure provides a method for visualizing a biomolecule of interest including contacting a biological sample with a first probe to produce a modified biological sample. The first probe has a first binding site capable of binding to a biomolecule of interest, and a plurality of second binding sites, each capable of binding to a detector oligonucleotide. The modified biological sample includes the first binding site of the first probe bound to the biomolecule of interest. The method for visualizing a biomolecule of interest further includes contacting the modified biological sample with one or more detector oligonucleotides to produce a complexed sample. Each detector oligonucleotide has a first binding site capable of binding to the second binding site of the first probe, and an enzyme. The complexed sample includes the first binding site of the first probe bound to the biomolecule of interest and the first binding site of each detector oligonucleotide bound to a second binding site of the first probes plurality of second binding sites. The method for visualizing a biomolecule of interest further includes contacting the biological sample with one more visualizing agents and visualizing the biomolecule of interest. The one or more visualizing agents are activated by the enzyme.

As used herein, the “biomolecule of interest” of this second or third aspect can be any biomolecule suitable for use according to the methods of the invention, including, but not limited to, RNA, DNA, or a protein,

As used herein, the “first probe” of this second or third aspect can be any probe suitable for use according to the methods of the invention, including, but not limited to, an oligonucleotide, an antibody, an aptamer, or lectin. In one non-limiting embodiment, the first probe comprises a probe conjugated to an oligonucleotide.

As used herein, the “detector oligonucleotide” of this third aspect can be any detector oligonucleotide suitable for use according to the methods of the invention, including, but not limited to, DNA, RNA, PNA, LNA, or morpholino.

As used herein, the “enzyme” can be any enzyme suitable for use according to the methods of the invention, including, but not limited to, horseradish peroxidase, ascorbate peroxidase, or microbial transglutaminase.

As used herein “one more visualizing agents” can include any substrate capable of being converted to a reactive visible form and suitable for use according to the methods of the invention. Non-limiting embodiments include small molecules (such as biotin, azide, alkyne, DIBO, DBCO), fluorophore labeled oligonucleotides, and/or tyramide compounds.

In various non-limiting embodiments, the substrate capable of being converted to a reactive visible form comprises a tyramide compound, biotin aniline, DAB (3, 3-diaminobenzidine), or salts thereof.

Non-limiting examples of a tyramide compound include biotin tyramide, cyanine tyramide, alkyne tyramide, and a fluorescent tyramide, or salts thereof.

As used herein, visualizing the biomolecule of interest can be accomplished by any suitable method including, but not limited to microscopy. In various embodiments, the microscopy can include any type of microscopy including, but not limited to, confocal, widefield, and super-resolution.

In one non-limiting embodiment of the third aspect, the first probe comprises an oligonucleotide probe, wherein the one or a plurality of second binding sites on the first probe are each capable of base pairing with the detector oligonucleotide.

In a further non-limiting embodiment, the first probe comprises a plurality of first probes. In this embodiment, the first binding site of each of the plurality of first probes binds to a different biomolecule of interest, and each second binding site of the first probe is capable of binding to a different detector oligonucleotide.

In a further non-limiting embodiment, the one or more detector oligonucleotides comprises a plurality of detector oligonucleotides. In this embodiment, the first binding site of each of the plurality of detector oligonucleotides is capable of binding to the second binding site of a different first probe.

In a further non-limiting embodiment, contacting the biological sample with the first probe comprises contacting the biological sample with the plurality of first probes to produce the modified biological sample.

In a further non-limiting embodiment, contacting the modified biological sample with the one or more detector oligonucleotides includes contacting the modified biological sample is with the plurality of detector oligonucleotides serially. This embodiment can include the following steps: (1) contacting the biological sample with one of the plurality of detector oligonucleotides to produce the complexed sample, (2) contacting the biological sample with one more visualizing agents, wherein the one or more visualizing agents are activated by the enzyme, (3) visualizing the one of the plurality of detector oligonucleotides with microscopy, (4) stripping the one of the plurality of detector oligonucleotides from the first probe, and (5) repeating steps (1)-(4) with a different one of the plurality of detector oligonucleotides. According to this embodiment, the contacting the biological sample with the plurality of first probes can include contacting the biological sample serially or simultaneously to the plurality of first probes.

As used herein “visualizing” can be accomplished by any suitable method including, but not limited to microscopy. In various embodiments the microscopy can include any type of microscopy including, but not limited to, confocal, widefield, and super-resolution.

As used herein “stripping” can be accomplished by any suitable method including, but not limited to adding a chemical denaturant, such as formamide or salt solution to the buffer, temperature changes, and/or via competitive displacement by toehold-mediated branch migration.

In a fourth aspect, the disclosure provides a kit including a first probe having a first binding site that binds to biomolecule of interest, and a plurality of second binding sites, each second binding site capable of binding to a detector oligonucleotide. The kit further includes a detector oligonucleotide having a first binding site capable of binding to the second binding site of the first probe, and an enzyme.

As used herein, the “first probe” of this fourth aspect can be any probe suitable for use with the kit, including, but not limited to, an oligonucleotide, an antibody, an aptamer, or lectin. In one, non-limiting embodiment, the first probe comprises a probe conjugated to an oligonucleotide.

In a fifth aspect, the disclosure provides a kit including a first oligonucleotide having a first binding site that binds to biomolecule of interest, and a plurality of second binding sites, each second binding site capable of base pairing with a second oligonucleotide. The kit further includes a second oligonucleotide having a first binding site capable of base pairing to the second binding site of the first oligonucleotide, and an enzyme.

According to this aspect of the disclosure, the enzyme can be any enzyme suitable for use with the kit, including, but not limited to horseradish peroxidase (HRP).

According to this aspect of the disclosure, the biomolecule of interest can be any biomolecule of interest suitable for use with the kit, including, but not limited to RNA, DNA, or a protein.

### EXAMPLES

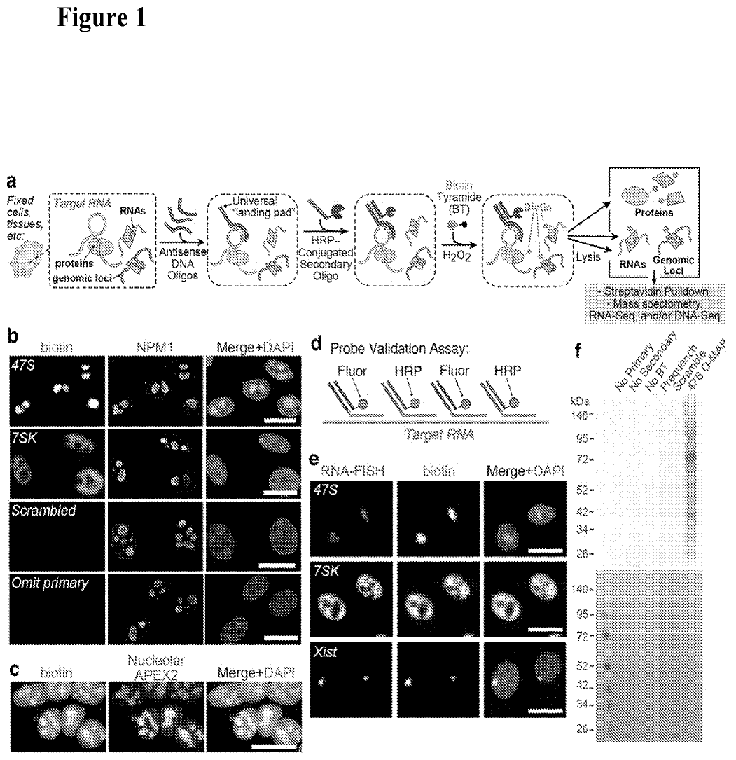

Presented herein is Oligonucleotide-directed proximity-interactome mapping (O-MAP), a straightforward and flexible method for applying proximity-labeling to individual RNA targets in genetically unmodified samples, using only off-the-shelf parts and standard manipulations. O-MAP utilized the same peroxidase/tyramide chemistry used in APEX-based proximity-omics approaches^{23, 24}, but it relied on programmable oligonucleotide probes, rather than transgenic expression, to deploy biotinylating enzymes to endogenous target RNAs (FIG. 1a). This approach enabled precise RNA-targeting that can be easily optimized and experimentally validated, overcoming a chief limitation of oligo-pulldown based approaches. Building on this, it was developed herein O-MAP mass-spectrometry (O-MAP-MS), RNA-Sequencing (O-MAP-Seq), and DNA-Sequencing (O-MAP-ChIP) workflows for unbiased, compartment-wide RNA-interaction discovery, using markedly fewer cells than established methods. Using these workflows, new interactions were discovered within the nucleolus and Barr bodies—RNA-scaffolded compartments that were difficult to isolate biochemically. Finally, it was demonstrated that O-MAP can be readily ported to different biological specimen types and to diverse classes of target RNA, demonstrating its utility as a broad-use RNA interaction-discovery tool. Overall, O-MAP's straightforward implementation and technical accessibility, its multi-omic analytical capabilities, and its flexibility across biological settings and target RNAs make this method readily adoptable by most molecular biology laboratories.

### Example 1: Materials and Methods

### Cultured cells, Tissue Sections and Organoids

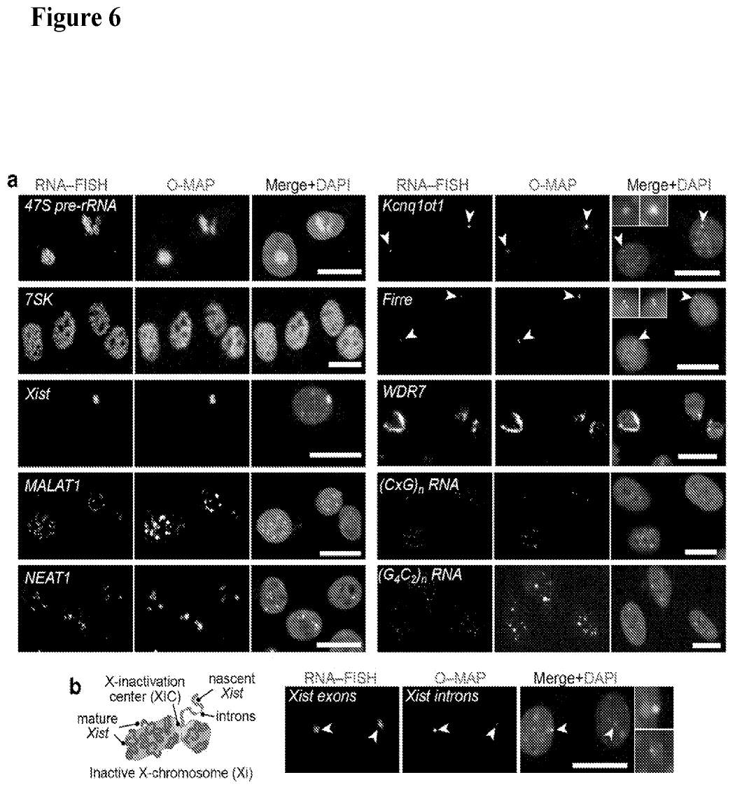

Hela cells, female mouse embryonic fibroblasts (fMEFs, a gift from Dr. C. Disteche, UW), HEK 293T, Patski, SUIT2, and U-2 OS cells were cultured in High Glucose DMEM with Pyruvate (Thermo Fisher; 11995073), supplemented with 10% (v/v) Fetal Bovine Serum (FBS, Thermo Fisher; 26140079), 100 units/mL penicillin and 100 μg/mL streptomycin (Thermo Fisher; 15140122), and 1x GlutaMAX™ (Thermo Fisher; 35050061). For Transgenic (CxG)ₙ and (G₄C₂)ₙ U-2 OS cells⁹⁸ (a gift from A. Jain, MIT) (FIG. 6a) qualified tetracycline-free FBS (Gibco; 26140079) was used. For these lines, transgenic RNA expression was induced by addition of doxycycline to a final concentration of 1 mg/mL for 24 hours prior to cell fixation. 8988T, A375, ASPCI, and Panc 3.27 cells were cultured in RPMI 1640 media (Thermo Fisher; 11875093), supplemented with 10% (v/v), FBS, 100 units/mL penicillin, and 100 μg/mL streptomycin. Patient-derived lymphoblast cell lines⁸⁸ (a gift from Dr. P. Valdmanis, UW) were cultured in IMDM (Thermo Fisher; 12440053), supplemented with 20% (v/v) FBS, 100 units/mL penicillin, 100 μg/mL streptomycin, and 2.5 μg/mL amphotericin B. In all cases, cells were maintained at 37° C., under 5% CO₂. Cell lines were authenticated by STR testing (ATCC), when possible.

For imaging experiments, cells were cultured in two-well Lab-Tek™ borosilicate glass #1.0 chambers (Thermo Fisher; 155380). To improve HEK293T adherence, chambers were treated with gelatin (0.5% (w/v), in water, Sigma; G7765) for 30 minutes at 37° C., prior to plating. For biochemistry, proteomic, and high-throughput sequencing experiments, cells were cultured in six-well plates. When necessary, material from multiple wells was harvested and merged into a single lysate, as described below.

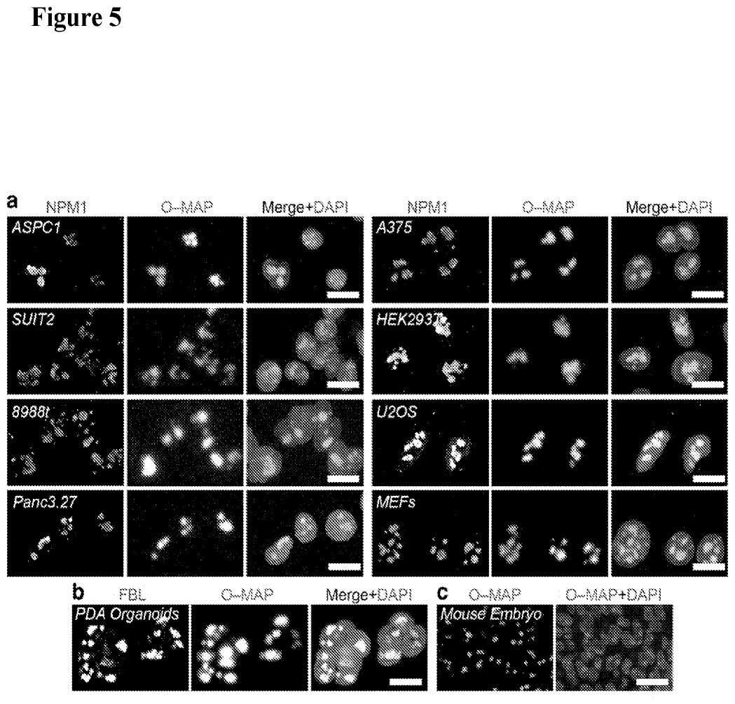

Human Pancreatic Ductal Adenocarcinoma organoids (FIG. 5b) were prepared as described⁸⁷. Mouse tissue sections (FIG. 5c, a gift from Dr. E. Nichols, UW) were prepared from day E13.5 embryos (C57BL/6J wild type mice; Jackson Laboratory) by drop fixing in 4% (v/v) formaldehyde, followed by sucrose equilibration, and thin-sectioning in OCT compound (VWR 25608-930. Cryosections (approximately 10 μm) were prepared at the UW Histology and Imaging Core and stored on glass slides at −80° C. until use.

### O-MAP Probe Design and Synthesis

Probes targeting the human 47S pre-rRNA ITSI domain were taken from (Ref. 37). All other probes were designed using OligoMiner™ pipeline³⁴ using the following settings. The blockParse script was run using the settings: −1 30-L 37-t 42-T 47-s 390-F 40. Bowtie2 was used with settings: -U--no-hd-t-k 100--very-sensitive-local-S. The outputClean script was run with the-u argument; the structure Check script was run with the settings: -F 40-s 390-m dna1998-T 42. K-mer filtering was performed in Jellyfish version 2.2.10, using a Jellyfish file for the corresponding genome (human or mouse), and using the kmerFilter function with the -m 18 and -k 1 arguments. Jellyfish files were generated for each genome assembly (hg38 for human; mm 10 for mouse), using a hash size set to the appropriate size of the genome assembly³⁴. For example, the command -s 3300M -m 18 -o hg38_18.jf-out-counter-len 1-L 2 hg38.fa was used to generate the 18mer dictionary for hg38. For most targets, all probes that passed this final filtering step—typically 10-150 probes per target—were used. For kcnq1ot1, a set of 200 k-mer-filtered probes were used.

For the O-MAP Probe Validation Assay (FIG. 1d), probes were divided into sub-pools along the length of the target RNA, appended on their 3′ termini with SABER1 or SABER2 “Landing-pad” sequences³⁵. Once probes were validated, the complete pool was reformulated, appended with SABER1 and used for O-MAP alone.

Probe sets consisting of fewer than 20 probes were ordered as individual oligos (Sigma; 0.025-0.05 μg synthesis scale, standard desalting), and further purified from preparative polyacrylamide gels, as described previously⁹⁹. Purified oligos were resuspended in nuclease-free water, quantified by UV-vis spectroscopy, pooled to a final aggregate concentration of 20 μM and stored at −20° C. Probe pools requiring more than 20 oligos were purchased as oPools™ (IDT; 50 pmol/oligo scale, unmodified), and dissolved to approximately 100 μM in nuclease free-water. 20 μL were desalted using the Oligo Clean & Concentrator Kit™ (Zymo Research; D4060), following the manufacturer's instructions. Pools were quantified by UV-vis spectroscopy, diluted to 5 μm and stored at −20° C. Fluorophore-conjugated secondary probe used in RNA-FISH (“SABER2-AF647”) was purchased from IDT (100 nmol scale; HPLC purification), resuspended to 100 μM in nuclease-free water, and stored in a light-tight container at −20° C. The HRP-conjugated secondary probe (“SABER1-HRP”) was purchased from Bio-Synthesis, resuspended to 10 μM in resuspension buffer (10 mM NaH₂PO₄, 150 mM NaCl, pH 7.2), allotted into 10 μL single-use aliquots, flash-frozen and stored at −20° C.

In some cases, the RNA-FISH signal was amplified by extending the FISH probe subpool with concatemers of additional “SABER2” Landing-pads. These were enzymatically added via the Polymerase Exchange Reaction (PER), essentially as described³⁵. Briefly, pooled probes (5 μM, aggregate) were incubated with 0.5 μM Template hairpin and 0.1 μM Clean.G DNA hairpin (IDT), in 10 mM MgSO₄, 0.6 mM each ATP, CTP, and TTP, 4 U Bst 2.0 DNA Polymerase (NEB; M0537), 1x PBS, in a final volume of 50 μL. Reactions were incubated at 37° C. for two hours in a thermocycler, heat-inactivated at 80° C. for 20 minutes, and chilled on ice. The resulting PER-extended oligos were then purified with Oligo Clean and Concentrator Kits™, eluting into nuclease-free water, and their length was examined on denaturing 10% Polyacrylamide TBE-Urea gels, stained with SYBR™-Gold (Thermo Fisher; S11494).

### O-MAP Core Protocol

The following protocol was used for omics-scale O-MAP, using cells grown in six-well dishes (3.5×10⁵ cells/well; plated one day prior to harvest). For imaging-only experiments, cells were plated at 7×10⁴ cells per well, in two-chamber LabTeks™. In all cases, RNase-free reagents and manipulations were used throughout.

The core O-MAP workflow is split over two days. The first day comprises fixation, permeabilization, peroxidase inactivation, and primary probe hybridization; the second day comprises secondary probe hybridization, an optional endogenous biotin blocking step, and in situ biotinylation. Thereafter, the protocol varies depending on the endpoint assay—imaging, proteomics, RNA-Seq, or ChIP-Seq.

O-MAP Day 1. All manipulations were performed at room temperature, unless noted. Cells were washed briefly with 1x Ca- and Mg-free DPBS (Thermo Fisher; 14190250) and fixed with freshly prepared 2% (v/v) formaldehyde (Electron Microscopy Sciences; 15710) in 1x PBS (Sigma; 6506), for 10 minutes without agitation. The formaldehyde solution was aspirated and the crosslinking reaction terminated by two washes with 250 mM glycine in 1x PBS, five minutes each, with gentle rocking (3 rpm on a platform rocker), Cells were briefly washed with DPBS, permeabilized with 0.5% (v/v) triton-X™ 100 in PBS (10 min; gentle rocking), and washed three times with DPBS. Next, to inactivate endogenous peroxidases, samples were treated with 0.5% (v/v) H₂O₂, in 1x PBS, for 10 minutes with gentle rocking, and washed twice with PBS. Samples were then equilibrated in Formamide Wash Buffer (10-40% (v/v) deionized formamide (Thermo Fisher; AM9342); 2x SSC (Thermo Fisher; AM9765); 0.1% (v/v) Tween™-20), for five minutes with gentle rocking. The formamide concentration was matched to the primary probe hybridization mix, as determined by the binding parameters of the primary probe pool. This buffer was then aspirated, and each sample was treated with 115 μL of Probe Mix (0.1 μM primary oligo probe pool, in 1x Hybridization Buffer: 10-40% deionized formamide; 2x SSC; 0.1% (v/v) Tween-20™; 10% (w/v) dextran sulfate (SIGMA; D8906); in nuclease-free water) and this mix was gently spread over the sample by covering with a clean, 30 mm diameter #1.5 thickness glass cover slip (Bioptechs; 30-1313-03192). A 2x SSC-soaked kimwipe was placed between the wells to maintain humidity during hybridization. Plates were then sealed with Parafilm and incubated without agitation for 8 hours at 37° C. or 42° C., depending on the probe set.

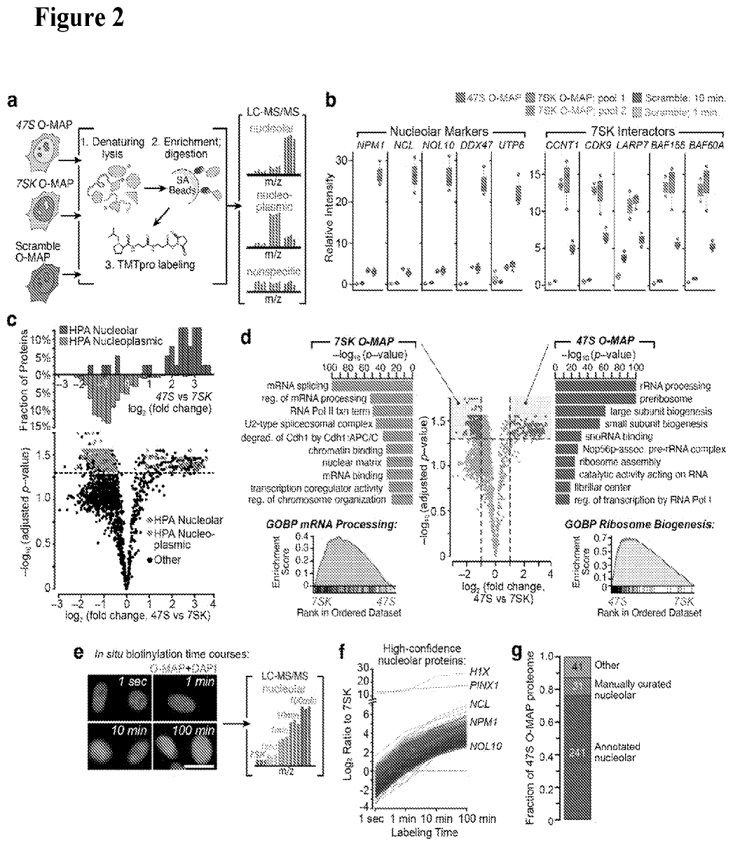

O-MAP Day 2. Following primary hybridization, coverslips were removed and cells were washed three times with pre-warmed 30% Formamide Wash Buffer, 10 minutes per wash, at 37° C. with gentle rocking. For imaging experiments, this was followed by a blocking step to mask endogenous biotinylated proteins, as described below (see “O-MAP Imaging”). In all cases, subsequent manipulations were carried out in the dark, to avoid photooxidation of the HRP conjugate. Each well was treated with 115 μL O-MAP Secondary Probe Mix (100 nM SABER1-HRP oligo, in 30% Formamide Hybridization Buffer), and covered with a clean coverslip. Samples were incubated for 1 hour at 37° C., with gentle rocking. Coverslips were then removed, and samples were washed four times with PBST (0.1% (v/v) Tween-20™ in 1x PBS), 15 minutes per wash, with gentle rocking. Buffer was aspirated, and in situ biotinylation induced by addition of 1 mL Labeling Solution (0.8 μM biotinyl-tyramide (Sigma; SML2135), 1 mM H₂O₂, 1x PBST), and incubation at room temperature. Labeling times varied between RNA targets—ranging from 1 second (FIG. 2e, top left) to 120 minutes—and were determined empirically using the O-MAP Imaging assays described below. In all cases, biotinylation was halted by addition of Sodium Azide and Sodium Ascorbate (10 mM each, final) in 1x PBST, for three washes of five minutes each.

### O-MAP Imaging

For imaging experiments, the background signal from endogenous biotinylated proteins was blocked after the secondary probe hybridization step. Briefly, samples were washed three times in 1x PBST and incubated in pre-blocking solution (1% (w/v) nuclease-free BSA (VWR; 97061-420) in 1x PBST) at room temperature for 30 min with gentle rocking. Samples were then blocked with 1 mL of Neutravidin™ Blocking Solution (10 μg/mL neutravidin™ (Thermo Fisher; 31000), 1% (w/v) nuclease-free BSA, in 1x PBST) for 15 min with gentle rocking at room temperature, and washed three times with PBST. To saturate free streptavidin binding sites, samples were next treated with 10 μg/mL D-biotin (Thermo Fisher; B20656) in 1x PBST, for 15 minutes with gentle rocking, followed by three washes with room temperature PBST. Thereafter, in situ biotinylation and quenching proceeded as described above, using 50 μL volumes for primary and secondary hybridization buffers. After biotinylation and quenching, samples were stained with 1 mL 1 μg/mL neutravidin-DyLight™ 550 conjugate (Thermo Fisher; 84606), in 1% BSA pre-blocking solution, for one hour at room temperature with gentle rocking, followed by three washes with 1x PBST. Samples were counterstained with DAPI (5 μg/mL, in 1x PBST) and imaged immediately, or were mounted in Vectashield (Vector Labs; H-1900-10) and stored at 4° C.

### O-MAP-MS

O-MAP-labeled cells (approximately 5.5×10⁶ cells per replicate; three replicates per experimental condition) were harvested by scraping into 1x PBST, supplemented with 10 mM Sodium Azide. After pelleting by centrifugation at 800×g for 10 minutes at 4° C., remaining buffer was aspirated and the pellets were flash frozen in liquid nitrogen and stored at −80° C. until use. All subsequent steps were performed at room temperature, unless noted. Cell pellets were lysed in 800 μL of MS Lysis Buffer (4% (w/v) SDS in 1x PBS, with 10 mM Sodium Azide, 1x Halt™ EDTA-Free Protease Inhibitor Cocktail) for five minutes. Samples were then sonicated using a Branson Digital Sonifier 250 outfitted with a double stepped microtip (Emerson Industrial Automation) at 10-12 Watts for 30 seconds (0.7 s on; 1.3 s off) for one cycle. Lysates were clarified by centrifugation at 15,000×g for 10 minutes and soluble protein was quantified using the Pierce BCA Protein Assay Kit™ (Thermo Fisher; 23225). For each sample, 300 μg of protein was diluted to 1% SDS by the addition of three volumes Dilution Buffer (1x PBS, supplemented with 10 mM Sodium Azide and 1x Protease Inhibitors). Protein samples were then reduced with TCEP™ (Thermo Fisher; 77720, 10 mM final concentration) for 60 minutes with gentle rotation. To alkylate free thiol groups, samples were treated with iodoacetamide (Sigma; 11149, 20 mM final concentration) and rotated for 60 minutes at room temperature, before quenching by addition of DTT (5 mM final) and incubation for 15 minutes. For streptavidin pulldown, to each sample was added 100 μL Pierce Streptavidin Magnetic Bead™ slurry (Thermo Fisher; 88817) that had been equilibrated in Diluted Lysis Buffer (1% (w/v) SDS, 1x PBS, 10 mM Sodium Azide, 1x Protease inhibitors). Samples were rocked end over end for two hours at room temperature, and streptavidin beads were then washed with the following buffers (5 minutes per wash; rocking end over end at room temperature): (1-2) Two washes in Diluted Lysis Buffer, (3) 1x PBS (4-5) Two washes in KCl Buffer (1 M KCl in 1xPBS), (6-7) Two washes in Urea Buffer (2 M Urea in 1xPBS), (8-9) Two washes in 200 mM EPPS (pH 8.5). Beads were then resuspended in 15 μL 200 mM EPPS (pH 8.5), and bound proteins were eluted by on-bead proteolytic digestion, as follows. Lysyl endopeptidase (Lys-C, Fujifilm Wako; 121-05063) was added at a ratio of 1 μg of enzyme per 100 μg of input protein, and samples were incubated for three hours at 37° C., with shaking at 500 rpm. Trypsin (Thermo Fisher; 90057) was then added at a ratio of 1 μg of enzyme per 100 μg of input protein, and digestion continued overnight at 37° C., 500 rpm shaking.

### O-MAP-MS LC-MS/MS Sample Preparation

Eluted peptides were labeled with TMTpro™ reagents using established protocols⁴⁹. Briefly, eluted peptides were supplemented to with acetonitrile to a final concentration of 30% (v/v), in 200 mM EPPS buffer (pH 8.5). TMTpro™M reagents in 100% anhydrous acetonitrile were then added to each sample at approximately a 2.5:1 (w/w) excess. The labeling reaction was allowed to continue for 1.5 hours, quenched with 5% hydroxylamine, and the labeled peptides were mixed. Pooled peptides were then dried by vacuum centrifugation. Dried, labeled peptides were resuspended in 100 μl of (5% acetonitrile, 1% formic acid) and cleaned using in-house assembled stage-tips¹⁰⁰. Pooled peptides were eluted in (70% acetonitrile, 1% formic acid). Eluates were then dried to completion and stored at −80° C. until analyzed by LC-MS/MS.

### O-MAP-MS Data Acquisition and Analysis

Pooled, labeled peptides were resuspended in (5% acetonitrile, 2% formic acid) and eluted over an in-house pulled 25 cm C18 column (Accucore, Thermo Fisher Scientific) throughout a 180 minute gradient from (6% acetonitrile, 0.125% formic acid) to (32% acetonitrile, 0.125% formic acid). Peptides were analyzed using an SPS-MS3™ method on a Thermo Fisher Orbitrap Eclipse™ to quantify TMTpro™ reporter ions. Briefly, the duty cycle consisted of three FAIMS™ (FAIMSpro, Thermo Fisher Scientific) mobility regions at Compensation Voltages (CV)=−40/−60/−80V. At each CV the following were collected within a duty cycle: an MS1™ scan (R=120,000, MaxIT=50 ms), six MS2™ scans (Ion trap, Turbo scan speed, MaxIT=50 ms, AGC=200%, CID NCE=35%), and six SPS-MS3™ scans (R=50,000, MaxIT=86 ms, HCD NCE=45%, AGC=400%). A single dynamic exclusion of 90s was used across all CVs.

Resulting spectra were analyzed using the Comet search algorithm™¹⁰¹, searched against a full human protein database with forward and reverse protein sequences (Uniprot™ 10/2020). Precursor monoisotopic peaks were estimated using the Monocle package¹⁰². Peptides and proteins were filtered to a 1% false discovery rate using the rules of parsimony and protein picking¹⁰³. Protein quantification was done using signal-to-noise estimates of reporter ions and these data were processed and plotted using the R statistical programming language. Gene Ontology analysis was performed using MetaScape™⁵⁵, and GSEA™⁵⁶.

### O-MAP-Seq

O-MAP-labeled cells (approximately 9×10⁶ cells—one six-well dish—per replicate; three biological replicates per experimental condition) were harvested by scraping into PBSTq™ (1x PBST, supplemented with 10 μM Sodium Azide, 10 μM sodium ascorbate) and pelleted by centrifugation at 3,000×g for 10 minutes. Buffer was aspirated and cells were flash frozen in liquid nitrogen and stored at −80° C. until use. Pellets were thawed on ice and resuspended by gentle pipetting in 1000 μL ice cold RIPA™ Buffer (50 mM Tris-HCl pH 7.5, 150 mM NaCl, 0.1% (w/v) SDS, 0.5% (w/v) Sodium Deoxycholate, 1% (v/v) Triton X-100, 5 mM EDTA, 0.5 mM DTT), supplemented with 1x EDTA-Free Halt™ Protease Inhibitor Cocktail, 0.1 U/μL RNase-OUT™ (Thermo Fisher; 10777019)), and 10 mM Sodium Azide, and rocked end-over-end at 4° C. for five minutes. Samples were then sheared using a Branson Digital Sonifier 250 outfitted with a double stepped microtip, at 10-12 Watts for 30 s intervals (0.7 s on; 1.3 s off), with 30 s resting steps between intervals, seven intervals total. Samples were held in ice-cold metal thermal blocks throughout sonication. Lysates were then clarified by centrifugation at 15,000×g for 10 min at 4° C., moved to fresh tubes and diluted with 1 mL Native Lysis Buffer (NLB: 25 mM Tris-HCl pH 7.5, 150 mM KCl, 0.5% (v/v) NP-40, 5 mM EDTA, 0.5 mM DTT), supplemented with 1X Halt™ Protease Inhibitor Cocktail, 0.1 U/μL RNase-OUT™ and 10 mM sodium azide. For each sample, 5% was removed as “input,” and to the remainder was added 100 μL of Pierce streptavidin magnetic bead slurry that had been equilibrated by two washes in 1:1 RIPA: NLB supplemented with 10 mM sodium azide, 0.1 U/μL RNase-OUT™, and 1X Halt Protease Inhibitor Cocktail. Samples were incubated for 2 hr at room temperature with end-over-end agitation. Beads were then washed with the following series of buffers (1 mL each, 5 min per wash at room-temperature with end-over-end agitation). All buffers were supplemented with 1x EDTA-Free Halt™ protease inhibitor cocktail, 0.1 U/μL RNase-OUT™, and 0.5 mM DTT, unless otherwise noted: (1) RIPA, supplemented with 10 mM Sodium Azide; (2) RIPA alone (3) High Salt Buffer (1 M KCl, 50 mM Tris-HCl pH 7.5, 5 mM EDTA) (4) Urea Buffer (2 M Urea, 50 mM Tris-HCl pH 7.5, 5 mM EDTA) (5) 1:1 RIPA: NLB, without protease inhibitors (6) NLB, without protease inhibitors, (7-8) two washes in TE buffer (10 mM Tris-HCl pH 7.5, 1 mM EDTA), without protease inhibitors.

RNA was isolated from both input and O-MAP-enriched samples by proteolysis in 100 μL Elution Buffer (2% (v/v) N-lauryl sarcoside, 10 mM EDTA, 5 mM DTT, 200 μg proteinase K (Thermo Fisher; AM2548), in 1x PBS). Reactions were shaken at 700 rpm in a Mixer HC (USA Scientific) for 1 hour at 42° C., followed by 1 hour at 60° C. RNA was then extracted once with 1 volume of phenol pH 4.3, and twice thereafter with an equal volume of absolute chloroform. Reactions were supplemented with 15 μg Glycoblue™ (Thermo Fisher; AM9515) and NaCl to 300 mM, and ethanol precipitated at −20° C. overnight. RNA was harvested by centrifugation at 15,000×g for 30 minutes at 4° C., washed twice with 70% ethanol, and resuspended in 84.75 μL nuclease free water. Contaminating DNA was removed by digestion with S U RQ1 RNase-free DNase I™ (Promega; M6101) in 100 μL of the manufacturer's supplied buffer (1x final concentration) at 37° C. for 30 min, and this reaction was terminated by addition of EDTA to 15 mM, final. RNA was purified by phenol extraction and ethanol precipitation, as described above, and resuspended in 15 μL nuclease free water. Sample concentration was measured using a Nanodrop One™ (Thermo Fisher)

### O-MAP-Seq Library Preparation and Sequencing

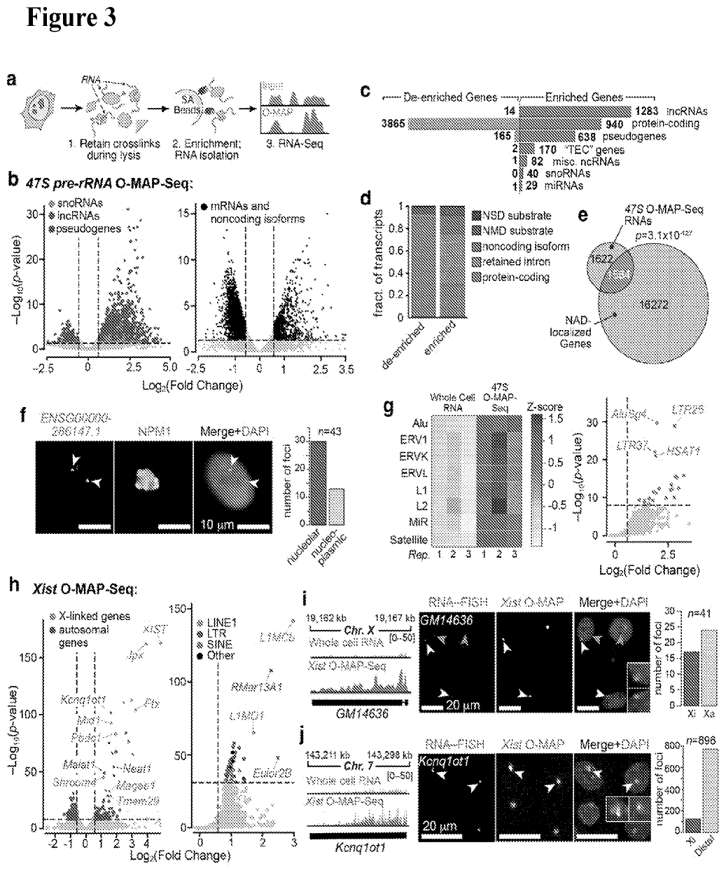

Ribosomal RNA was first depleted by RNase-H digestion, using pools of antisense DNA oligonucleotides that targeted mature rRNAs, but not the pre-rRNA “transcribed spacer” domains⁶⁵ as described previously⁶⁵. For Patski cells (FIG. 3h-j), rRNA was removed using a NEBNext™ rRNA Depletion Kit (NEB; E6310), according to the manufacturer's protocol, and RNA was further purified using acidic phenol extraction, chloroform cleanup, and ethanol precipitation. The antisense oligos used for HeLa cells (FIG. 3a-g), described previously⁶⁵, were synthesized as an oPool™ (IDT; 50 pmol per oligo, unmodified), desalted using a Zymo OligoClean™ and Concentrator Kit, following the manufacturer's instructions, and resuspended into nuclease-free water. 1 μg of antisense probes were added to 1 μg of RNA (whole cell, or O-MAP-enriched), in 200 mM NaCl, 100 mM Tris-HCl, pH 7.4, at a final volume 10 μL. This solution was heated to 95° C. for 2 minutes and then slowly cooled to 45° C. at a rate of −0.1° C./s, using a ProFlex™ PCR system (Thermo Fisher). Reactions were then supplemented with 10 μL of preheated RNase H mix (10 U Hybridase Thermostable RNase H (Lucigen; H39500), 20 mM MgCl₂). Reactions were incubated at 45° C. for 30 minutes and placed on ice. RNA was then purified by acidic phenol-chloroform extraction and ethanol precipitation, residual DNA was removed using RQ1 DNase, and RNA was again purified by phenol-chloroform extraction and ethanol precipitation, as described above.

Each sample was quantified on a Nanodrop One™ (Thermo Fisher). Sequencing libraries were prepared from 300 ng RNA using the NEBNext™ Ultra II Directional RNA Library Prep Kit and NEBNext™ Multiplex Oligos for Illumina (NEB; E7760 and E7735), according to the manufacturer's instructions. Three biological replicates were used for each experimental condition; each library was given a unique index. Libraries were quantified using the NEBNext™ Library Quant Kit for Illumina (NEB; E7360), following the manufacturer's instructions, and the quality of these libraries was confirmed using an Agilent 4200 TapeStation with an “DNA High Sensitivity” kit (Fred Huch Genomics Core). Libraries were pooled in equimolar concentrations to 20 nM aggregate concentration in nuclease-free water, with no more than 12 libraries per pool. These were subjected to 150 cycles of paired-end sequencing, followed by indexing, on one lane of an Illumina HiSeq™ 4000 per pool, run in high output mode (Azenta Life Sciences).

### O-MAP-Seq Data Analysis

For gene-and isoform-specific expression analyses (FIGS. 3b-d and 3h, left), raw RNA-seq FASTATM files were aligned to reference genomes using HISAT2 version 2.2.1, in the paired-end setting with default parameters¹⁰⁴ For 47S-O-MAP, reads were mapped to a modified GRCh38 genome assembly (courtesy of T. Moss, Université Laval) in which all rDNA repeats are replaced by a single copy of the consensus rDNA locus as an extra chromosome (“GRCh38_rDNA”)¹⁰⁵. For XIST-O-MAP, reads were mapped to mm10. The resulting SAM files were converted to BAM format and sorted using Samtools¹⁰⁶ version 1.15.1. Bigwig files for visualizing strand-specific information were created using deep Tools¹⁰⁷ version 3.5.1 with parameters: --filterRNAstrand forward/reverse --binSize 1--normalizeusingBPM. Mapped reads were quantified using String Tie™¹⁰⁸ version 2.2.1 and the String Tie output was prepared for differential expression analysis using the prepDE.py function. The resulting gene and transcript count matrices were used for differential expression analysis using DESeq2 (Ref.¹⁰⁹) with a FDR cutoff of 0.05.

Transposable element (TE) expression analysis (FIGS. 3g and h, right) was performed using the TEtranscripts pipeline¹¹⁰. Briefly, raw RNA-seq fasta files were mapped to GRCh38 (for 47S) or mm10 (for XIST), using STAR¹¹¹ version 2.7.10a, allowing multi-mapped reads with the following settings: --sjdbOverhang 149-win Anchor Multimap Nmax 200-out Filter Multimap Nmax 100-out SAM type BAM Unsorted. TE expression was then quantified using TEtranscripts¹¹⁰ version 2.2.1, with the following parameters: --stranded reverse --mode multi --minread 1 --padj 0.05-i 100. Differentially expressed TEs were identified using DESeq2 version 1.34.0 with a FDR cutoff of 0.05.

Volcano plots (FIGS. 2c,d; 3b,g,h; 17; 19) were generated using Enhanced Volcano version 1.12.0. All statistical analysis (Fisher's exact test, hypergeometric distribution test, or Student's t-test, where appropriate) was performed in R or in python using the ggplot2 (ggplot2.tidyverse.org/), or seaborn¹¹² and matplotlib¹¹³ modules.

### O-MAP-ChIP

O-MAP-labeled cells (approximately 5×10⁶ per replicate; two biological replicates per experimental condition) were harvested in PBSTq (1x PBST, supplemented with 10 μM Sodium Azide, 10 μM sodium ascorbate) by scraping, and pelleted by centrifugation at 3,000×g for 10 minutes. Buffer was aspirated and cells were frozen in liquid nitrogen and stored at −80° C. until use. Pellets were thawed on ice and gently resuspended by pipetting in 1 mL CLB (20 mM Tris pH 8.0, 85 mM KCl, 0.5% (v/v) NP-40), supplemented with 1x Halt™ EDTA-Free protease inhibitor cocktail and 10 mM Sodium Azide, for 10 minutes. Lysates were then clarified by centrifugation at 3,000×g for five minutes at 4° C. Supernatant was aspirated, and samples were subjected to another round of CLB extraction, clarification, and supernatant aspiration. Pellets were then lysed by gentle pipetting in 1 mL of NLB (10 mM Tris-HCl pH 7.5, 1% (v/v) NP-40, 0.5% (w/v) Sodium Deoxycholate, 0.1% (w/v) SDS) and incubated on ice for 10 minutes. Samples were then sheared using a Branson Digital Sonifier outfitted with a double stepped microtip, at 10-12 Watts over 30 s intervals (0.7 s on; 1.3 s off), with 30 s resting steps between intervals, 18 intervals total. This resulted in an average shearing size of approximately 200 bp, as gauged on an agarose gel. Samples were held in ice-cold metal thermal blocks throughout sonication. Lysates were then clarified by centrifugation at 15,000×g for 10 minutes at 4° C. and supernatants were moved to fresh tubes. For each sample, 10% was removed as ‘input’; to the remainder was added 100 μL of streptavidin-coated magnetic bead slurry that had been equilibrated by two washes in NLB. Samples were incubated for 2 hours at room-temperature with end-over-end agitation. Beads were subsequently washed with the following series of buffers (1 mL each, 5 minutes per wash, at room-temperature, with gentle end-over-end agitation): (1) NLB, supplemented with 5 mM EDTA, 10 mM Sodium Azide and protease inhibitors (1x Halt™ EDTA-free Protease Inhibitor Cocktail), 150 mM NaCl; (2) NLB, supplemented with 5 mM EDTA, 10 mM Sodium Azide and protease inhibitors, (3-4) two washes in 1 M KCl, 10 mM Tris-HCl pH 7.5, 5 mM EDTA, (5-6) two washes in 2 M Urea, 10 mM Tris-HCl PH 7.5, 5 mM EDTA, (7) 10 mM Tris-HCl PH 7.5, 1% (w/v) SDS, (8-9) 10 mM Tris-HCl pH 7.5, 1 mM EDTA.

DNA was isolated from both input and enriched samples by proteolysis in 100 uL of Elution Buffer (2% (v/v) N-lauryl Sarcoside, 10 mM EDTA, 5 mM DTT, in 1x PBS, supplemented with 200 μg proteinase K). Samples were shaken for 1 hour at 700 rpm in a Mixer HC at 65° C. Supernatants were transferred to 0.2 mL tubes and incubated at 65° C. overnight in a thermocycler. DNA was then extracted with an equal volume of phenol pH 6.6, followed by two extractions in equal volumes of absolute chloroform. Samples were supplemented with 1 μg GlycoBlue and NaCl to 300 mM final, and ethanol precipitated at −20° C. overnight. DNA was harvested by centrifugation at 15,000×g for 30 minutes at 4° C., washed twice with 70% ethanol, and resuspended into 20 uL nuclease free water. To remove residual RNA, each sample was supplemented with 10 μg RNase Cocktail Enzyme Mix™ (Thermo Fisher; AM2286) and incubated at 37° C. for 1 hour. DNA was then purified by phenol extraction and ethanol precipitation as described above and re-suspended in 20 μL nuclease-free water.

### O-MAP-ChIP Library Preparation and Sequencing

DNA samples were quantified using a NanoDrop One™. 300 ng DNA of each sample was used for library preparation, using the NEBNext™ Ultra II DNA Library Prep Kit and NEBNext™ Multiplex Oligos for Illumina (NEB; E7645 and E7335), according to the manufacturer's instructions. Two biological replicates were used per experimental condition; each library was given a unique index during synthesis. Library concentrations were measured using the NEBNext™ Library Quant Kit for Illumina, and the quality of each sample was confirmed using an Agilent 4200 TapeStation™ with a “DNA High Sensitivity” kit (Fred Hutch Genomics Core). Libraries were pooled in equimolar concentrations to 20 nM aggregate concentration in nuclease-free water, with no more than eight libraries per pool. These were subjected to 150 cycles of paired-end sequencing, followed by indexing, on one lane an Illumina HiSeq™ 4000 per pool, run in high output mode (Azenta Life Sciences).

### O-MAP-ChIP Data Analysis

Deep sequencing reads were trimmed using TrimGalore! ™ (www.bioinformatics.babraham.ac.uk/projects/trim_galore/) with parameters-q 30--phred33, and mapped to the appropriate reference genome using Bowtie2 version 2.4.4 (Ref. ¹¹⁴). For 47S-O-MAP-ChIP, reads were mapped to GRCH38_rDNA¹⁰⁵; for XIST, reads were mapped to mm 10. Duplicate reads were removed with the Picard MarkDuplicate™s function (broadinstitute.github.io/picard) before peak calling. O-MAP-ChIP data were normalized to replicate-matched input samples. For 47S-O-MAP, nucleolar associated domains were called by merging peaks from Enriched Domain Detector™ (EDD)¹¹⁵ and epic2™ (Ref. ¹¹⁶). EDD™ was performed using default settings and—because NADs are enriched for highly repetitive sequences like centromeres—an empty BED file for the blacklist region. Epic2 peaks were called with the settings --bin-size 50000-g 2. Peaks from EDD™ and epic2 were first concatenated and then merged with the BEDtools™¹¹⁷ merge function, using the default settings. For XIST-O-MAP, regions of enrichment were called using MACS2 (Ref. ¹¹⁸) with the broadPeak setting. Further statistical analysis was perfomed in R or python, as described for O-MAP-Seq, above.

Epigenomic analysis of NADs was performed using ChromHMM™ version 1.22. The OverlapEnrichment™ function was called using the E117_25_imputed12marks_hg38lift_segments.bed file from the RoadMap™ Epigenomics Project and the final NAD calls in BED file format. Raw enrichment of each epigenomic signature for each cell line was plotted as a heatmap using seabor version 0.10.1.

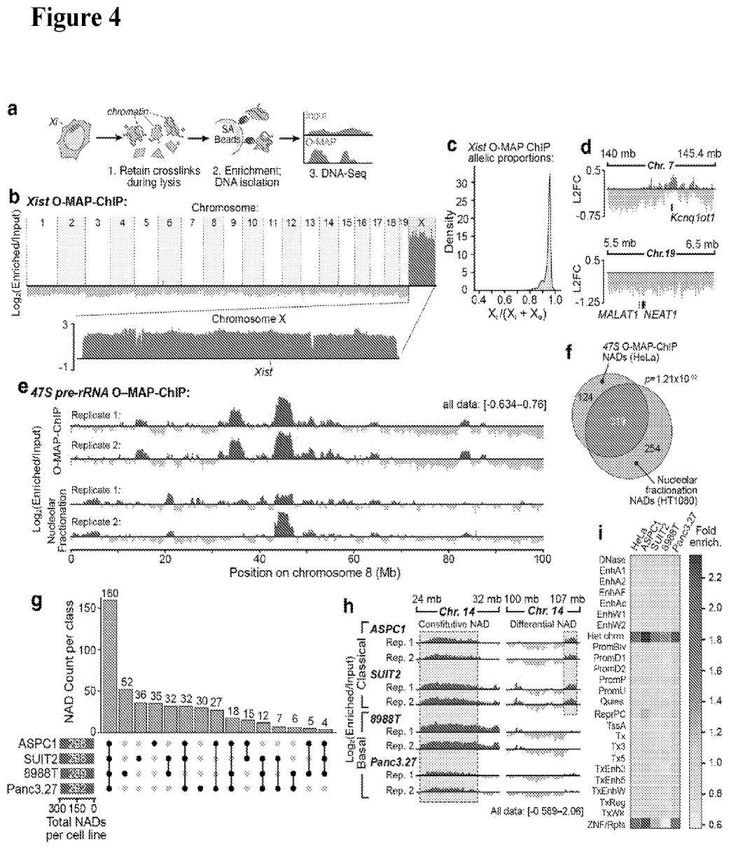

SNP analysis of the allelic segregation of XIST-interacting chromatin regions (FIG. 4b) was performed as previously described, using a method optimized for the Patski cell line¹¹⁹. Briefly, analysis was restricted to MACS2-enriched regions along chromosome X with a coverage of at least five reads. Each region's allelic proportion was then calculated by computing (Xᵢ/(Xₐ+Xᵢ)) for each read within the region. Regions with an allelic proportion >0.7 were designated as Xᵢ-specific; those with an allelic proportion <0.3 were designated as Xₐ-specific; those between 0.3 and 0.7 were classified as common to both alleles.

### Streptavidin Blotting

O-MAP labeled samples were lysed, sonicated, and clarified as described for O-MAP-MS. Samples were quantified by BCA, supplemented with 2× laemmli loading buffer and heated to 95° C. for 10 minutes. Samples, standardized by protein mass, were loaded and separated on 10% SDS-PAGE gels, transferred onto PVDF membranes and stained with Ponceau S™ (Sigma P7170). Membranes were blocked with 5% (w/v) powdered milk in TBS-T (20 mM Tris, pH 7.5, 150 mM NaCl, 0.1% (v/v) Tween™-20) for one hour with rocking at room temperature. Membranes were washed three times in TBS-T, 5 minutes per wash, and blotted with streptavidin-HRP conjugate (Thermo Fisher S911, diluted 1:20,000 in TBS-T, supplemented with 5% (w/v) BSA) overnight at 4° C. Blots were washed three times for five minutes in TBS-T, developed using the SuperSigna™l West Pico PLUS Chemiluminescent Kit (Thermo Fisher 34580), and imaged.

### RNA-FISH, O-MAP Probe-Validation Assays, and Immunofluorescence

For RNA-FISH, cells were fixed in formaldehyde, permeabilized with Triton™-X 100, equilibrated in formamide wash buffer, and hybridized to primary probes as described above (see: O-MAP Day 1), but omitting the peroxidase inactivation step. Thereafter, samples were washed three times in 30% Formamide Wash Buffer (five minutes per wash; room temperature) and incubated with 50 μL FISH Secondary Probe Mix (100 nM SABER2-AF647 oligo, in 30% Formamide Hybridization Buffer), and covered with a clean coverslip in a hybridization chamber. Samples were incubated for 1 bour at 37° C. in the dark. Samples were washed three times with PBST (five minutes per wash), counterstained with DAPI solution (5 μg/mL, in 1x PBST) and either imaged immediately, or stored sealed in vectashield at 4° C.

For combined O-MAP and RNA-FISH experiments (FIGS. 1d-e, 3i-j, and 6), including the Probe-Validation Assay (FIG. 1d), RNA-FISH signal was dramatically diminished when HRP-and Fluorophore-conjugated secondary oligos were hybridized simultaneously, presumably due to fluorophore damage during the in situ biotinylation reaction. To avoid this, O-MAP and RNA-FISH were performed sequentially. O-MAP was performed first, as described above (see: O-MAP Core Protocol), including the endogenous biotin blocking step (see: O-MAP Imaging). After quenching the O-MAP biotinylation reaction, samples were washed three times in 30% Formamide Wash Buffer (5 minutes per wash; room temperature), and incubated with 50 μL FISH Secondary Probe Mix (100 nM SABER2-AF647, in 30% Formamide Hybridization Buffer) for 1 hour at 37° C. Samples were washed four times with PBST (five minutes per wash), stained with neutravidin™-fluorophore conjugate, counterstained with DAPI solution for two minutes, and either imaged immediately in 1x PBST or mounted in vectashield.

When combined with immunofluorescence (FIGS. 1b, 3f, and 5a-b), O-MAP or RNA-FISH were performed prior to immunostaining. Cells were subjected to the RNA-FISH or O-MAP pipelines, as described above. Following secondary probe hybridization (RNA-FISH) or in situ biotinylation and quenching (O-MAP), cells were washed three times in 1x PBST, and then blocked with 5% (w/v) nuclease-free BSA (VWR 0332) in 1x PBST for one hour at room temperature. Samples were then incubated with rabbit anti-NPMI (Thermo Fisher; PA517742, used at 1:100 dilution) or mouse anti-FBL (Thermo Fisher; MA1-22000; used at 1:100 dilution), in 1% (w/v) BSA, 1x PBST, for one hour at room temperature with gentle rocking. Samples were washed four times with 1x PBST and then incubated with either AlexaFluor™ 488-conjugated anti-rabbit (Thermo Fisher; A32731TR, 1:1000 dilution), or AlexaFluor™ 488-conjugated anti-mouse (Thermo Fisher; A32723TR. 1:1000 dilution), with neutravidin™-DyLight™ 550 conjugate (Thermo Fisher; 84606, 1:1000 dilution) as appropriate, in 1% (w/v) nuclease-free BSA, 1x PBST, for one hour at room temperature. Samples were washed four times with 1X PBST (15 minutes per wash), counterstained with DAPI for two minutes, and either imaged immediately in 1x PBST or mounted in vectashield.

Fluorescence widefield microscopy was performed on a Leica™ DM IL, equipped with a HC Fluotar™ 100x oil immersion objective with a 1.32 numerical aperture and planar correction (Leic™a; 11506527), a white LED light source (Leica™; EL6000) and a DFC365 FX digital camera (Leica™, 11547004). The following filter cubes were used: Texas Red (Leica™ TX2 ET; 11504180; used with Dylight™-550 conjugates), Cy5 (Leica™ YS ET; 11504181, used for Alexafluor-647), GFP (Leica GFP ET; 11504174, used for Alexa Fluor-488), and DAPI (Leica DAPI ET; 11504204). Illumination intensity was adjusted using the light source manual control; acquisition times ranged from 40-2000 ms, as controlled by the Leica™ LASX™ software. Fluorescence confocal microscopy was performed on a Leica™ SP8X microscope (UW Keck Imaging Center), outfitted with a HC CS2 63x off immersion objective, with 1.40 numerical aperture with both planar and apochromatic correction. The average voxel size was 0.06×0.06×0.346 μm. Samples were illuminated using a 470-670 nm tunable White Light Laser system, with a typical laser power of 0.1% for DAPI, 3% for 550 nm, and 30% for 647 nm. Gain and offset settings were adjusted to avoid pixel saturation. Images were line-averaged twice, with an average pixel dwell time of 1.58 us. A bit-depth of 8 or 16 was used and zoom factor between 1-3 was used for all images.

### Image processing

Images were processed using Fiji™¹²⁰ and ImageJ™¹²¹, and multicolor overlays were made using the screen setting in Adobe Photoshop³⁵. Most confocal images are maximum projections of z-stacks; the remainder correspond to single z-slices. Brightness and contrast were adjusted for display purposes using Fiji™ and ImageJ™ or Adobe Photoshop™. In all cases, contrast adjustment was applied to improve signal visibility, by changing the minimum (black) and maximum (white) values only. Automated despeckling was applied when necessary (e.g. in RNA-FISH images with weak, diffuse speckling in between cells) to reduce residual background signal. Colocalization analysis (FIG. 3f,i,j) was performed using Fiji¹²².

### Example 2: O-MAP Design and Implementation

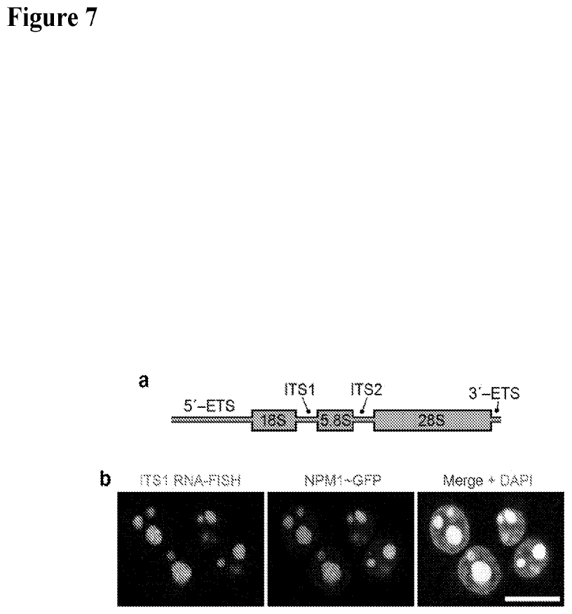

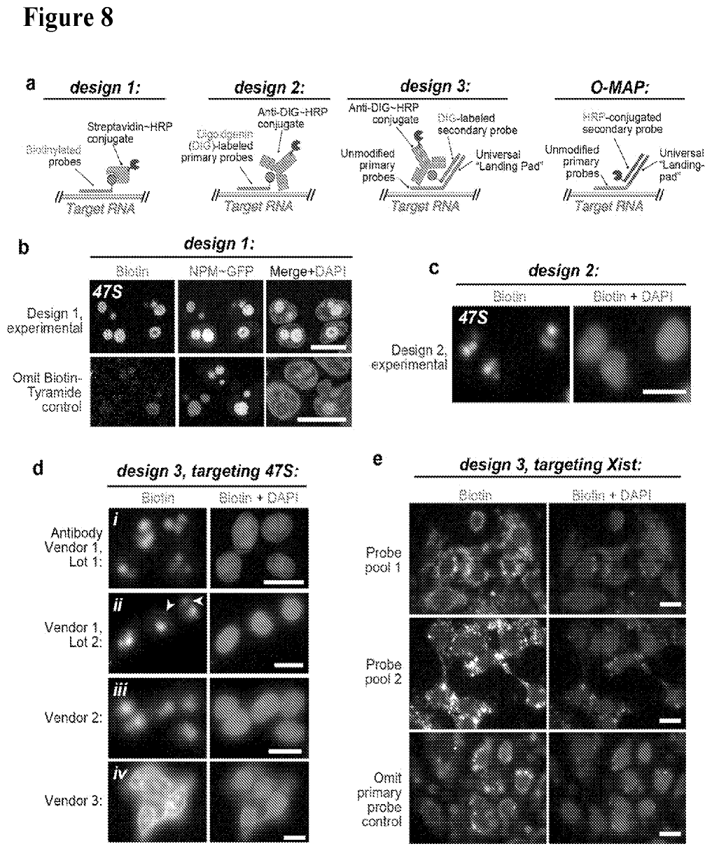

To specifically target the 47S precursor (and avoid mature ribosomes) an established RNA-FISH probe set³⁷ against ITS1, a “transcribed spacer” domain that is degraded during ribosome biogenesis and which never departs from the nucleolus (FIG. 7)³⁸ was selected. These probes were then used to explore different HRP recruitment strategies, by appending them with functional modules that can be targeted by HRP conjugates, and scoring the resulting nucleolar biotinylation by neutravidin staining (FIG. 8). Several of these strategies used small-molecule haptens (e.g. Digoxigenin) to recruit an HRP-conjugated hapten-binding protein—similar to classical Tyramide Signal Amplification (TSA)-FISH and related interaction-discovery methods^{26,30,31,39,40}. Yet, while these strategies induced prominent nucleolar biotinylation, they also exhibited variable off-target labeling that frequently rivaled the nucleolar signal (FIG. 8).

In contrast, the claimed optimized O-MAP strategy relied exclusively on programable oligonucleotide hybridization to precisely target HRP to an RNA of interest (FIG. 1a). In O-MAP, cells or tissues were chemically fixed with formaldehyde, and pools of oligo probes were annealed to the target RNA. These probes were chemically unmodified, but were appended with universal “landing pad” sequences originally developed for the FISH techniques Oligopaint⁴¹ and SABER³⁵. In a subsequent hybridization step, these landing pads recruited a common secondary oligo that is conjugated to HRP⁴². Upon addition of biotin-tyramide and hydrogen peroxide, HRP generated short-lived, highly reactive phenoxyl radicals that diffused outward and pervasively biotinylate molecules near (˜10 nm) the target RNA²⁴, enabled their enrichment.

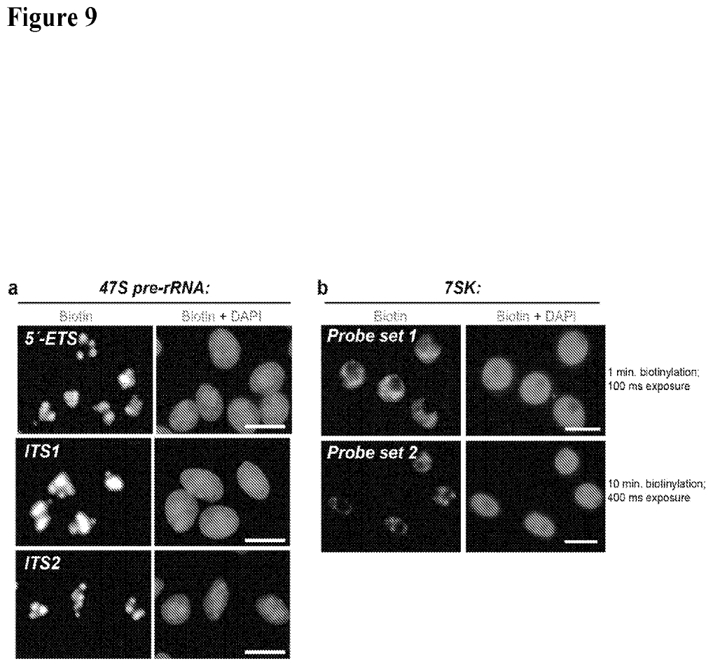

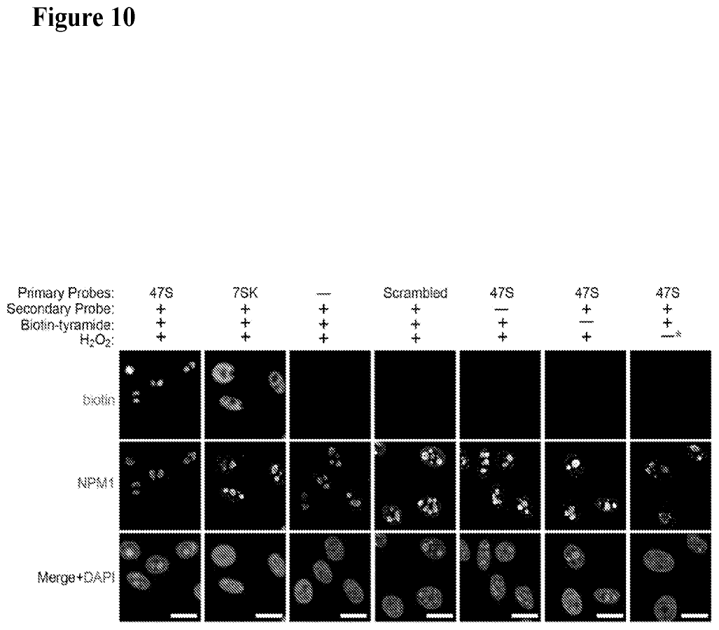

Unlike hapten-based HRP-recruitment strategies (FIG. 8), 47S-targeted O-MAP induced prominent and highly precise nucleolar biotinylation with nearly undetectable background (FIG. 1b). Likewise, O-MAP targeting 7SK—a small noncoding RNA thought to reside in nuclear speckles⁴³—produced exclusively nucleoplasmic labeling. For each RNA, similar results were observed using multiple distinct probe sets, including probes designed using the OligoMiner Pipeline³⁴ to have different hybridization parameters (FIG. 9). Importantly, omitting any component of the O-MAP workflow (primary oligos, the oligo-HRP conjugate, HRP substrates), or using scrambled primary probes, completely ablated biotinylation (FIG. 1b and FIG. 10). Moreover, 47S-targeted O-MAP appeared markedly more spatially refined than the analogous genetically encoded approach—nucleolar-targeted APEX2^{25, 44}—which generated substantial off-target, nucleoplasmic biotinylation (FIG. 1c).

O-MAP's landing pad design also enabled a straightforward strategy for optimizing the specificity of its probe pool, thus overcoming a longstanding challenge of oligo pulldown-based approaches¹⁴⁻¹⁷ (FIG. 1d). The claimed O-MAP Probe Validation Assay first grouped probes into “odd” and “even” sub-pools. Each sub-pool was outfitted with a distinct landing pad that recruited either an HRP-conjugated or fluorescent secondary oligo, enabling O-MAP and RNA-FISH to be performed simultaneously. Off-targeting (i.e. non-colocalizing) probes were then readily identified and eliminated. This assay was applied to the 47S- and 7SK-targeting probe sets, and in both cases observed strong overlap between the O-MAP and FISH signals, further underscoring O-MAP's precision (FIG. 1e). This assay was then tested on a less abundant RNA with a more confined localization. For this, Xist, the long noncoding RNA (IncRNA) that drives X-chromosome inactivation, and which coats the inactive X-chromosome (Xi)⁴⁵ was chosen. As predicted, Xist O-MAP induced strong biotinylation in a single prominent nuclear punctum, and which exclusively co-localized with the Xist RNA-FISH signal (FIG. 1e).

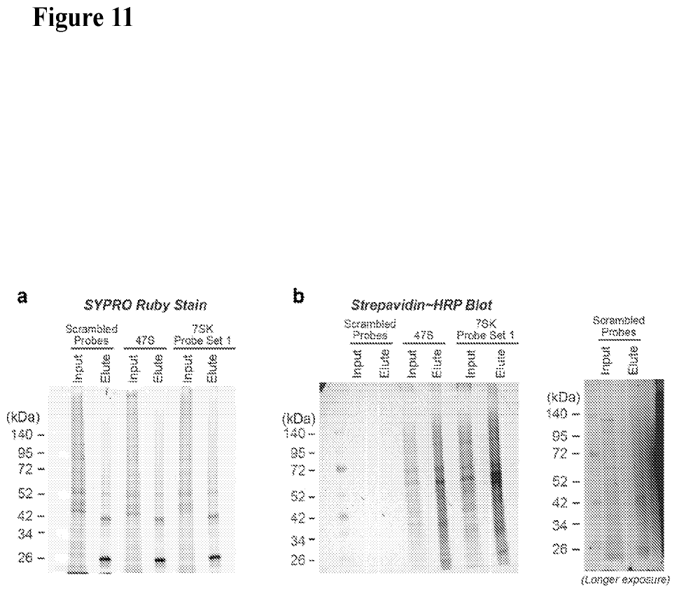

Of concern was that O-MAP's use of formaldehyde crosslinking and formamide denaturation might interfere with the solubility and recovery of biotinylated material. To examine this, 47S O-MAP to label nucleoli in Hela cells, and generated whole-cell lysates by boiling these cells in SDS was used. Streptavidin blotting of these samples revealed a prominent ladder of solubilized, biotinylated proteins, as would be expected from the diverse nucleolar proteome^{46, 47} (FIG. 1f and FIG. 11. Taken with results above, this demonstrates that O-MAP can generate exceptionally precise, RNA-targeted in situ biotinylation in a manner compatible with recovery and downstream omic analysis. A suite of tools for discovering proteins (O-MAP-MS), transcripts (O-MAP-Seq), and genomic loci (O-MAP-ChIP) interacting with a target RNA was therefore developed.

### Example 3: Mapping RNA-Proximal Proteomes with O-MAP-MS

To develop the O-MAP-MS pipeline, the primary test case was the nucleolus, the subnuclear organelle that compartmentalizes and controls ribosome biogenesis, in HeLa cells^{11,36}. The approach emulated the ratiometric quantification strategy used in APEX-based proximity-proteomics, which enhances precision by measuring the relative abundances of peptides within a target compartment and the adjoining subcellular space⁴⁸ (FIG. 2a). In parallel experiments, 47S O-MAP to label nucleoli, two independent 7SK-targeting probe sets (FIG. 9) as proxies for the neighboring nucleoplasmic compartment, and scrambled probes to model nonspecific background biotinylation was used. After O-MAP labeling, biotinylated proteins were enriched under denaturing conditions, and peptide abundances were measured by 16-plex Tandem Mass Tag (TMTPro) quantitative mass spectrometry⁴⁹. This multiplexed quantification strategy exploits the reversibility of formaldehyde crosslinks to yield chemically taggable primary amines, and would be challenging to implement with competing techniques that rely on irreversible fixatives³¹. It was also hypothesized that O-MAP-MS might require less starting material than established RNA interaction-discovery methods^{17,50}, since tyramide radical labeling amplifies the number of biotin moieties per RNA-bound HRP³⁹, enhancing capture efficiency. To test this, the initial proof-of-principle experiments employed only 5.5 million cells per replicate—approximately 20-100-fold less material than is needed for nucleolar fractionation⁵¹ or the oligo pulldown method ChIRP-MS¹⁷.

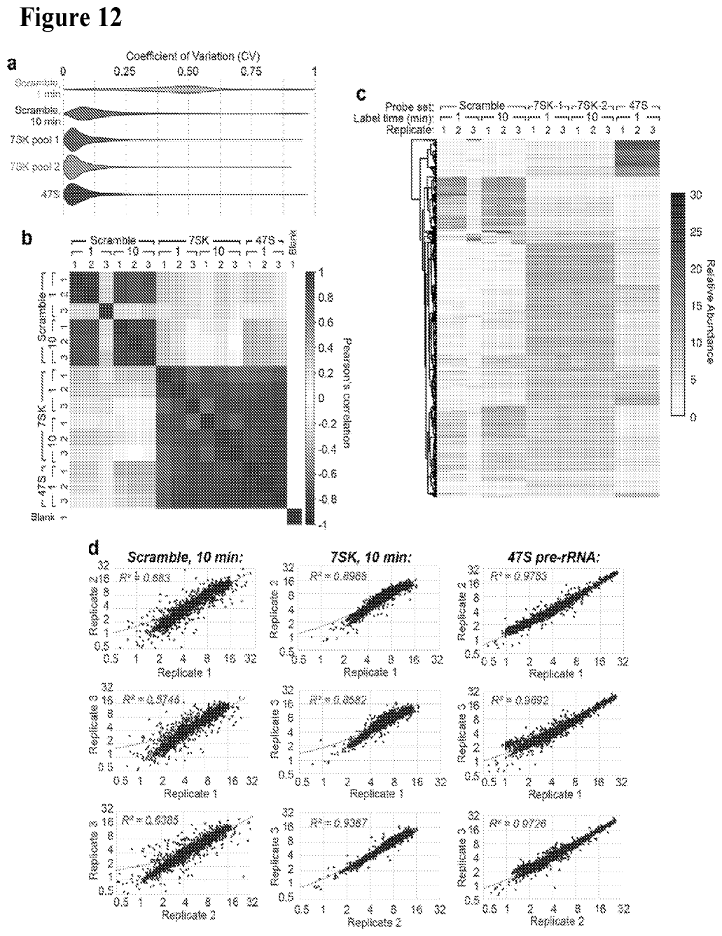

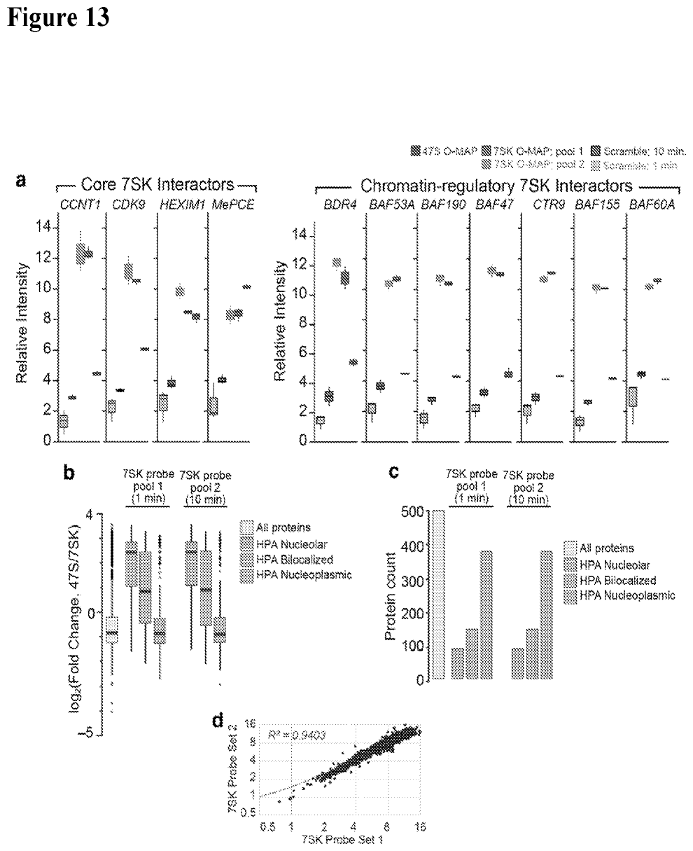

Yet, even with this small input mass, O-MAP-MS was able to accurately capture its target subcellular compartments at considerable depth (1855 total proteins detected) and reproducibility (Pearson's correlations ranging from 0.77-0.99 between replicates; FIG. 12). The data also demonstrated O-MAP's high spatial precision. For example, stereotypic nucleolar markers (e.g. NPM1, NCL), were robustly enriched by 47S O-MAP, and significantly less enriched by 7SK O-MAP or scrambled controls (FIG. 2b, left). This is consistent with the observation that many predominantly nucleolar proteins also partially localize to the nucleoplasm⁴⁷. Conversely, 7SK O-MAP highly enriched both classical components of the 7SK RNP (e.g. LARP7; Cyclin T1)⁵², and recently discovered interactors like BAF155 and BAF160A⁵³, further underscoring O-MAP's accuracy (FIG. 2b, right, and FIG. 13). Expanding on this analysis, more extensive sets of nucleolar (47S-proximal) and nucleoplasmic (7SK-proximal) marker proteins, using the Human Protein Atlas⁵⁴ (HPA), to define markers that are strictly confined to each compartment (38 strictly nucleolar and 414 strictly nucleoplasmic proteins observed in our data) were examined. This revealed a striking enrichment of resident nucleolar proteins exclusively from 47S O-MAP-MS (31 proteins, 81.5% of observed nucleolar markers; average 6.26-fold enrichment), and converse enrichment of resident nucleoplasmic proteins exclusively from 7SK O-MAP-MS (145 proteins, 35% of observed nucleoplasmic markers; average 2,62-fold enrichment), with a nearly 10-fold average separation between the two protein groups (FIG. 2c).

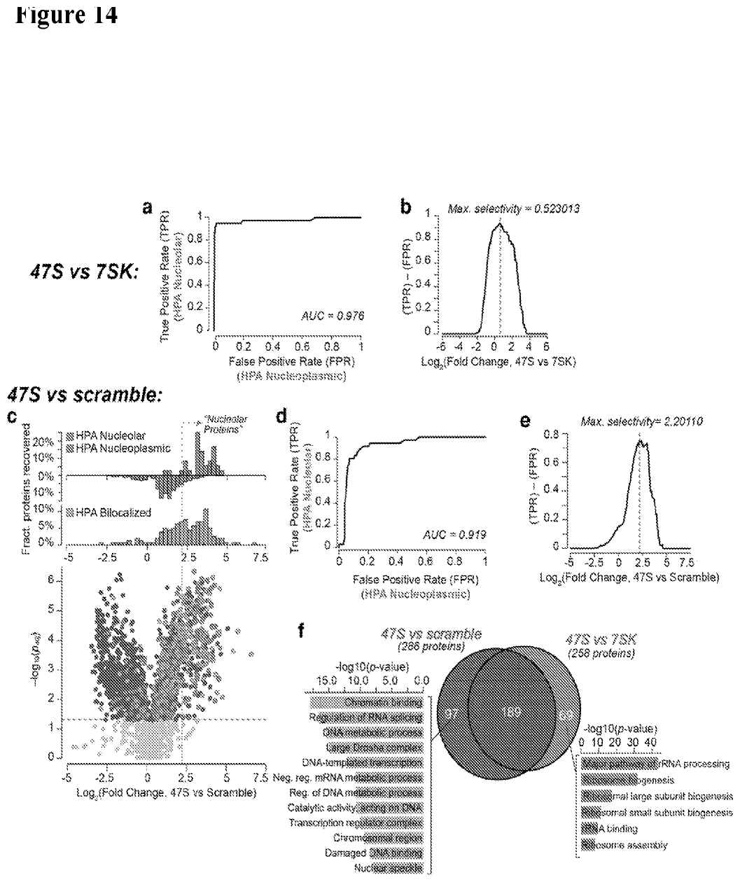

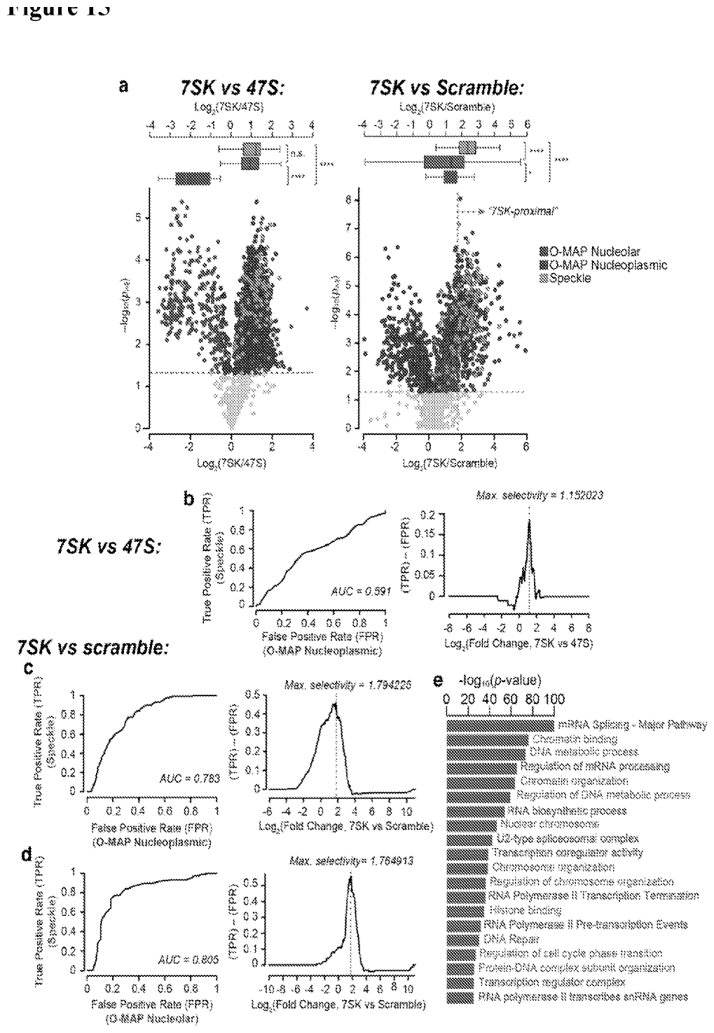

To further assess O-MAP's spatial precision, hypothesis-unbiased analysis of the putative 47S- and 7SK-proximal proteomes identified by our data was performed. These were defined as all statistically significant (p_{adj}≥0.05, t-test) proteins enriched at least twofold at either O-MAP target, relative to one another (i.e. log₂(47S/7SK) or log₂(7SK/47S)>1.0; FIG. 2d). Gene Ontology (GO)⁵⁵ and Gene Set Enrichment Analysis (GSEA)⁵⁶ of the putative 47S-proximal proteins (169, total) revealed a high enrichment of factors driving the transcription and processing of pre-ribosomal RNA, ribosome assembly, and nucleolar architecture, as predicted (FIG. 2d, right and FIG. 14). Likewise, the putative 7SK-proximal proteins (307, total) were highly enriched for factors involved in RNA Polymerase II regulation and mRNA biogenesis, consistent with 7SK's regulatory role in transcription elongation⁵² (FIG. 2d, left). The 7SK O-MAP data were also highly enriched in factors involved in pre-mRNA splicing, spliceosome assembly, and higher-order chromatin organization. Given that 7SK has been suggested to accumulate in nuclear speckles-membrane-less nuclear bodies in which spliceosomal components and splicing factors are also enriched^{43, 57}—it was hypothesized that 7SK O-MAP had specifically probed the speckle proteome. Indeed, when the set of proteins enriched from 7SK O-MAP relative to the scramble negative control (FIG. 15) was examined, it was observed that 127 (90%) of the 141 human speckle factors recently identified by protein-targeted proximity-biotinylation⁴⁰, were significantly enriched. This affirmed 7SK's localization to nuclear speckles, and further demonstrated O-MAP-MS's depth and precision, even with small-scale samples.

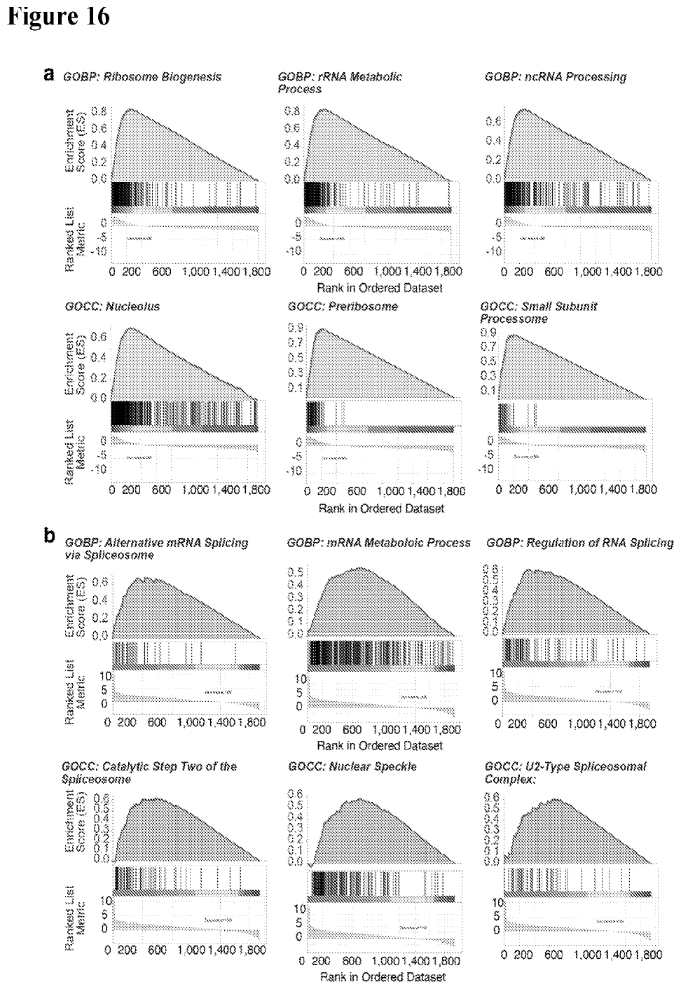

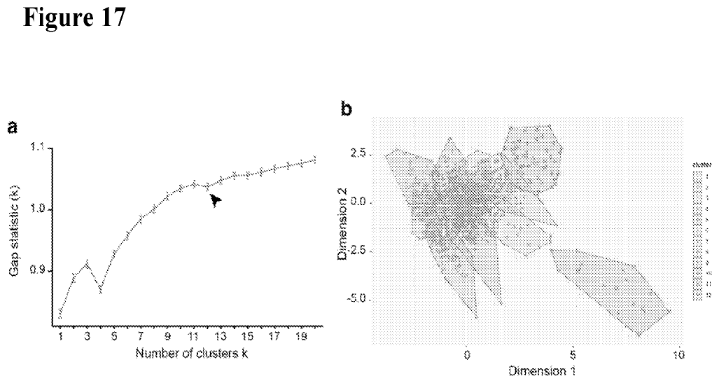

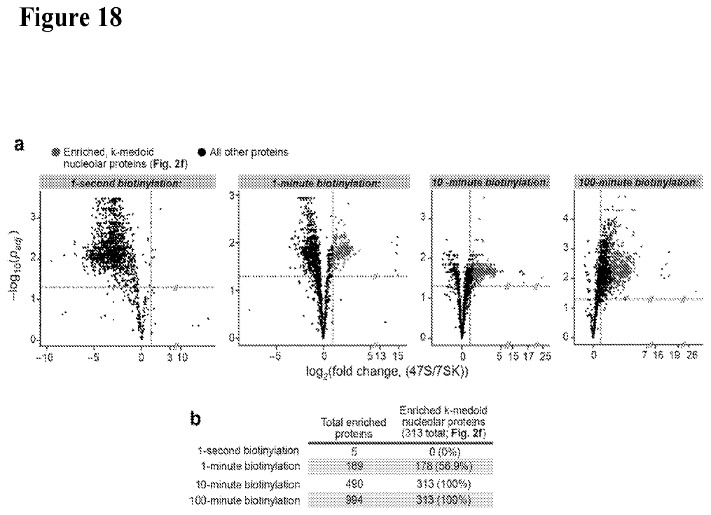

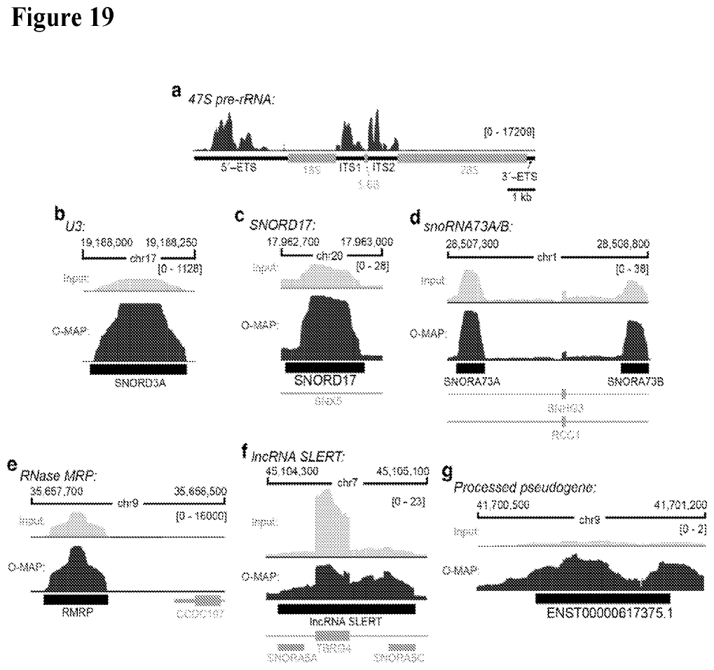

As a final proof-of-principle experiment, it was sought to use O-MAP to probe the nucleolar proteome at greater depth, by exploiting biotinylation time as a variable. It was reasoned that, during a labeling time course, proteins within a target compartment would be biotinylated with distinct kinetics from those adjoining it, allowing us to identify richer sets of compartment-specific factors by classifying common kinetic profiles. To test this hypothesis, 47S-targeted O-MAP at times ranging from 1 second to 100 minutes, using a single 7SK time point as a normalization control (FIG. 2e) was performed. Proteins by 16-plex TMTpro mass spectrometry, and used K-medoid analysis to cluster proteins based on their enrichment profile over time were quantified. This yielded twelve clusters, of which four appeared highly enriched for HPA-defined nucleolar proteins (FIG. 16) and which were merged to yield a list of 313 high confidence nucleolar proteins (FIG. 2f). Recovery of these proteins spike after one minute of in situ biotinylation and plateau by 10 minutes (FIG. 17). This cluster recovered approximately 90% (151, out of 168 proteins) of the 47S-proximal proteome observed in the first-pass analysis (FIG. 2d). Moreover, 241 (77%) proteins in this cluster have established roles in nucleolar organization or ribosome biogenesis, as annotated by the GO, HPA and Uniprot databases⁵⁸ (FIG. 2f). Manual curation of the remaining factors furthermore revealed 31 (10%) that also likely play roles in these processes, but which have been misannotated (FIG. 2f). This demonstrates the potential power of this kinetic clustering approach—which would be intractable by live-cell proximity-labeling due to the confounding effects of diffusion⁵⁹—to enable deeper analysis of the targeted compartment. Taken with the above, these data demonstrate O-MAP's ability to discover RNA-proximal proteomes with high precision.

### Example 4: Mapping RNA-Proximal Transcriptomes with O-MAP-Seq

Having established O-MAP as an RNA-targeted proteomic tool, it was next sought to expand the technique to transcriptomic analysis—mapping the transcripts localized near a target RNA. However, because tyramide-radical chemistry is markedly less efficient at labeling nucleic acids than proteins²⁶, it was anticipated that the direct capture of in situ-biotinylated RNA would be challenging, and require large-scale input cell growths²⁶. Therefore, a strategy based on APEX-RIP, in which formaldehyde crosslinks are retained during cell lysis and enrichment, and RNAs within the target compartment are captured by pulling down the biotinylated proteins to which they're covalently bound²³ (FIG. 3a) was adopted. The high capture efficiency of this approach enabled precise map RNA-proximal transcriptomes from as few as 8.3×10⁶ cells, approximately 12-24-fold lower than that required by oligo-capture-based methods^{14, 60}.

As a first test case, O-MAP-Seq to the HeLa nucleolus, using the 47S-targeting probe set established above, was applied (FIG. 1). The nucleolar transcriptome is thought to be predominantly noncoding⁶¹, comprising RNAs that regulate ribosome biogenesis (e.g. small nucleolar RNAs, snoRNAs)⁶², transcripts with putative nucleolar-architectural and chromatin-regulatory roles⁶³, and a large cohort of long noncoding RNAs (IncRNAs) with yet-undefined functions²⁵. Collectively, these transcripts arise from all three RNA polymerases and span lengths from 20 nucleotides⁶⁴ to several kilobases. It was attempted to capture these diverse species by using a non-poly (A)-selective, rRNA-depletion-based library preparation strategy similar to that established previously⁶⁵. This yielded a catalog of 47S-proximal RNAs that recapitulated much of the known nucleolar transcriptome, underscoring O-MAP-Seq's precision and accuracy (FIGS. 3b-c, 18, 19, and Table 1). Of note, conspicuous enrichment of every snoRNA detectable in our libraries (40 total, average 1.98 fold enrichment, p=0.011; FDR 0.05), the “transcribed spacer” domains within the 47S pre-rRNA itself³⁸, and nucleolar IncRNAs like SLERT⁶⁶ was observed. As predicted, coding genes were broadly de-enriched, with only 940 (4.64% of detectable genes) exhibiting significant nucleolar enrichment (FIG. 3b-c; p_{adj}=0.009; FDR 0.05). Moreover, isoform-level analysis revealed that the majority of these nucleolar-enriched, nominally protein-coding transcripts were in fact noncoding isoforms—most prominently, transcripts with retained introns (˜46% of enriched transcripts, comparted to ˜4% of the de-enriched pool, FIG. 3d). A similar intron-retention phenomenon has been recently observed in other subnuclear compartments⁶⁷, suggesting that the observations represented bona fide biological regulation and not artifacts of the O-MAP-Seq pipeline. Likewise, other genomic signatures of 47S O-MAP-Seq-enriched transcripts—notably, their genomic localization and domain architecture—suggested their validity as nucleolar RNAs. Nearly half (49.1%) of these transcripts are expressed from genes located within Nucleolar Associated chromatin Domains (NADs, FIG. 3e), compared to 7.7% of all genes. A similar enrichment of NAD-encoded transcripts was observed previously²⁵, suggesting that these RNAs may be localized to the nucleolus cotranscriptionally. Nucleolar transcripts were also enriched for nearly every family of transposable element (TE), with the AluSg4 family of Alu SINEs being the most significant (FIG. 3f, p_{adj}=2.22×10⁻³⁰; FDR 0.05). This is consistent with previous work demonstrating that Alu repeats are particularly enriched in nucleolar-and lamina-associated RNAs²⁵, and that these RNAs may play a role in stabilizing nucleolar architecture⁶⁸. Finally, the 47S O-MAP-Seq data also identified 3186 putative novel nucleolar transcripts (Table 1). It was confirmed by RNA-FISH that one such transcript, ENSG00000286147.1 (9.85-fold nucleolar enrichment; p_{adj}=0.048), exhibited nucleolar and perinucleolar localization (69.77%, n=43; FIG. 3g). This further validated O-MAP-Seq's accuracy and demonstrated its ability to discover novel RNAs within a target subcellular compartment.

TABLE 1
O-MAP Hybridization and Labeling Parameters

| | | Primary | | | |
|---|---|---|---|---|---|
| | | hybridization | Primary | | |
| | | buffer | hybridization | Biotinylating | |
| | Number of | formamide | temperature | Labeling | |
| Target RNA | Probes* | percentage (v/v) | (° C.) | Time | |
| 47S | 6 | 10 | 37 | 1 | min |
| 7sk probe | 9 | 30 | 37 | 1 | min |
| set 1 | | | | | |
| 7sk probe | 4 | 40 | 42 | 10 | min |
| set 2 | | | | | |
| Xist | 72 | 40 | 42 | 10 | min |
| MALAT1 | 151 | 40 | 42 | 10 | min |
| NEAT1 | 309 | 40 | 42 | 10 | min |
| Firre | 101 | 40 | 42 | 80 | min |
| Kcnq1ot1 | 200 | 40 | 42 | 40 | min |
| MS2** | 18 | 40 | 42 | 10 | min |
| WDR7 | 100 | 40 | 42 | 10 | min |
| pre-Xist*** | 104 | 40 | 42 | 60 | min |

*Probe number used in an O-MAP-only experiment, using the complete probe pool.
Probe Validation Assays used roughly half this number, both for O-MAP and for RNA-FISH.
**Probes use to target the tandem repeat model RNAs
***The Xist nascent transcript.

Encouraged by these results, it was next sought to apply O-MAP-Seq to a lower-abundance target with a previously uncharacterized RNA interactome. For this the IncRNA Xist, the “master regulator” of mammalian X-chromosome inactivation (XCI)⁴⁵ was chosen. In female embryos, monoallelic expression of Xist is sufficient to commit its copy of the X-chromosome to XCI, inducing widespread heterochromatinization that silences most genes on that chromosome⁶⁹⁻⁷¹. Differentiated cells typically express only 100-200 copies of the Xist transcript⁷²; these maintain epigenetic silencing by physically coating the inactive X-chromosome (Xi) and compacting it into a discrete subnuclear compartment⁷³. The protein-and chromatin-interactions that enable Xist to drive this process have been extensively studied^{69, 70}, but the RNA composition of the Xist-coated Xi compartment remains uncharacterized.

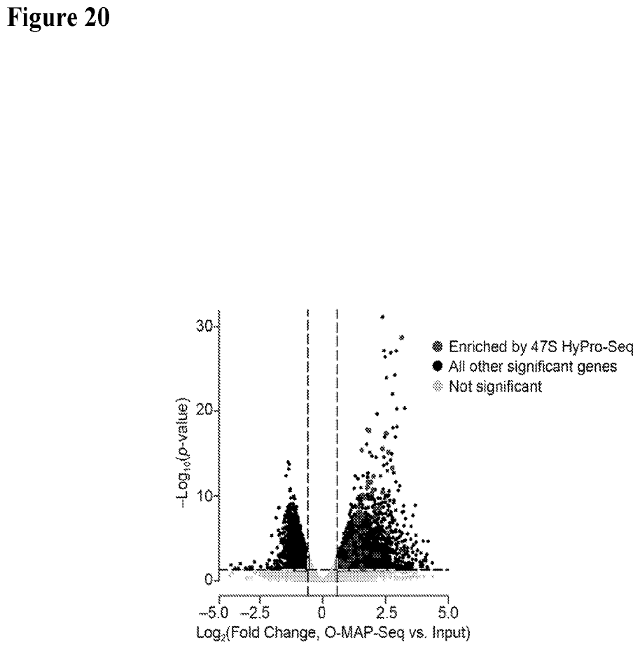

It was sought to elucidate the Xi transcriptome by O-MAP-Seq, using the validated Xist-targeting probe set (FIG. 1d) and the optimized pipeline developed above. For this mouse “Patski” cells, a classic cell-culture model system for Xist function⁷⁴ were chosen. As above, the data demonstreated that O-MAP-Seq had captured the local transcriptome near Xist with high precision and accuracy. Most strikingly, substantial enrichment of the Xist transcript itself, and of IncRNAs Jpx and Ftx (average 12.1-fold enrichment), which are located from the same genomic neighborhood and which also contribute to XC1⁷⁰ (FIG. 3h,left) was observed. Furthermore, prominent enrichment of several X-linked genes that are known to escape XCI (e.g. Mid1; Shroom4)⁷¹, likely due to the capture of nascent transcripts expressed from the Xi (FIG. 20) was observed. This was paralleled by the enrichment of the recently discovered noncoding RNA⁷⁵ GM14636, suggesting that it too might escape XCI (FIG. 3i, left), RNA-FISH analysis supported this hypothesis: though mono- and biallelic expression of GM14636 was variable in Patski cells, approximately 41.5% of observed foci co-localized with the Xist “cloud,” indicating penetrant expression from the Xi (FIG. 3i, right). Finally, it was noted that Xist O-MAP transcripts were highly enriched for several classes of X-linked transposable elements—most notably, the LIMCb and LIMD1 subfamilies of LINE1 elements (FIG. 3h, right). These elements are highly overrepresented on the X-chromosome, and have been hypothesized to play a role in XC1⁷⁶.

Intriguingly, Xist O-MAP-Seq also revealed the unexpected enrichment of several notorious chromatin-regulatory IncRNAs, including the imprinting regulator Kcnq1ot1 (FIGS. 3h and 3j, left). Much like Xist, Kcnq1ot1 is monoallelically expressed and physically coats an adjoining Mb-scale domain in cis, ultimately driving that domain's heterochromatinization and silencing⁷⁷. Remarkably, RNA FISH confirmed that Kcnq1ot1 co-localizes with the Xist “cloud” at higher-than-expected frequency (14% of puncta, n=896; FIG. 3j, right). This might indicate that Xist and Kcnq1ot1 shared a common pool of resources, or visited a shared subnuclear structure, that supported both of their epigenetic silencing functions. It furthermore highlighted O-MAP-Seq's ability to discover novel biological interactions at the “subcellular neighborhood” scale that have eluded prior detection.

### Example 5: Mapping RNA-Proximal Genomic Interactions with O-MAP-ChIP

The efficiency and precision of O-MAP-Seq led to the hypothesis that a similar approach could be used to probe RNA-DNA interactions—mapping the chromatin loci within a target transcript's subnuclear compartment. As with O-MAP-Seq, the strategy relied on formaldehyde crosslinking and the capture of in situ biotinylated proteins to enrich nearby DNA, similar to Chromatin Immunoprecipitation (ChIP; FIG. 4a). To develop this O-MAP-ChIP pipeline, it was first focused on the IncRNA Xist, in mouse Patski cells. In differentiated cells like these, Xist is thought to physically coat the inactive X-chromosome⁴⁵, and should thus exhibit a conspicuous and unambiguous ChIP signature. Indeed, when the Xist O-MAP-ChIP data genome-wide was mapped, a robust enrichment along the entirety of chromosome X (FIG. 4b) was observed. Patski cells are a hybrid line derived from a cross between M. musculus and M. spretus, and they exhibit skewed XCI in which the musculus X-chromosome is constitutively inactivated⁷⁴. This allowed for the quantification of the allelic segregation of Xist O-MAP-ChIP reads, by measuring distinct SNPs between maternal and paternal alleles. Strikingly, the average allelic proportions across Xist O-MAP X-chromosomal peaks were highly specific to the inactive (musculus) allele (94%), further underscoring O-MAP-CHIP's spatial precision (FIG. 4c).

Considerable evidence indicates that the Xist RNA makes direct chromatin contact exclusively with loci on the inactive X-Chromsome^{78, 79}. There was hence curiousity about the autosomal peaks in the O-MAP-ChIP data which, while unlikely to be points of direct interaction with the free Xist transcript, might correspond to rare, trans-chromosomal contacts near the Xist-bound Xi. A similar profile of autosomal contacts has been observed by oligo-capture methods upon Xist overexpression, suggesting that O-MAP may be able to capture weaker- or longer-range interactions for which other methods require signal amplification⁸⁰. One such putative trans-chromosomal interaction with the Kcnq1ot1 locus (FIG. 4d, top), which is thought to be directly bound by the Kcnq1ot1 RNA⁷⁷ was observed. Significant enrichment for this RNA by Xist O-MAP-Seq (FIGS. 3h,j) was also observed and implies that the Kcnq1ot1 locus—bound by its own transcript—is sometimes recruited near Xist-bound loci on the Xi. Importantly, the chromatin-regulatory IncRNAs Malat1 and Neat1, which were highly enriched at the RNA level by Xist O-MAP-Seq (FIG. 3h) but which are not thought to bind their own loci¹⁸, were not enriched at the DNA level by Xist O-MAP-ChIP (FIG. 4d, bottom).

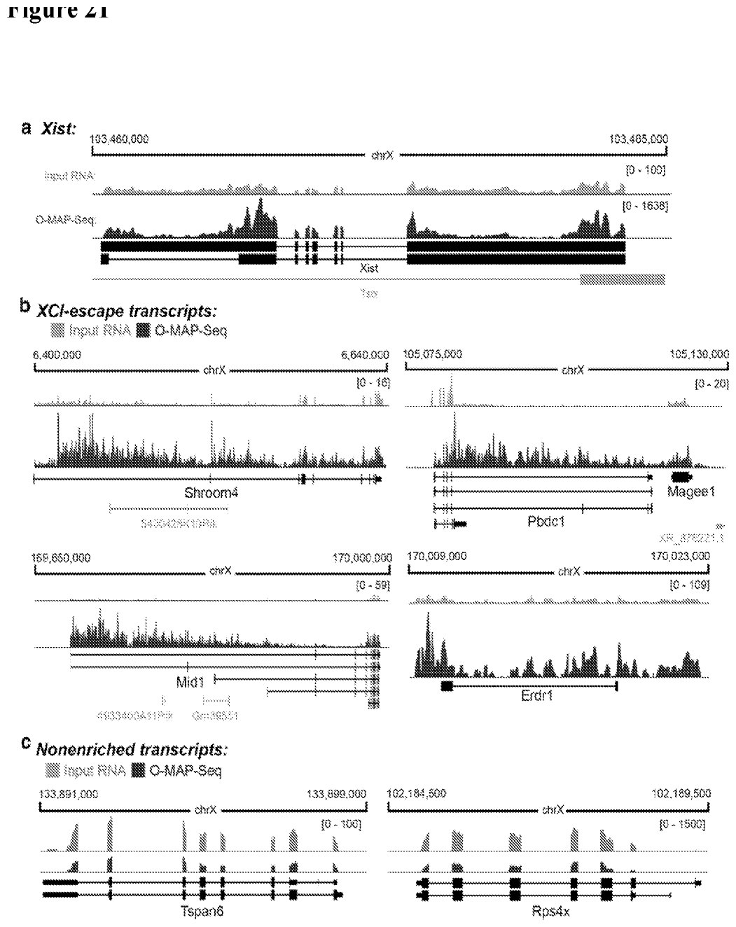

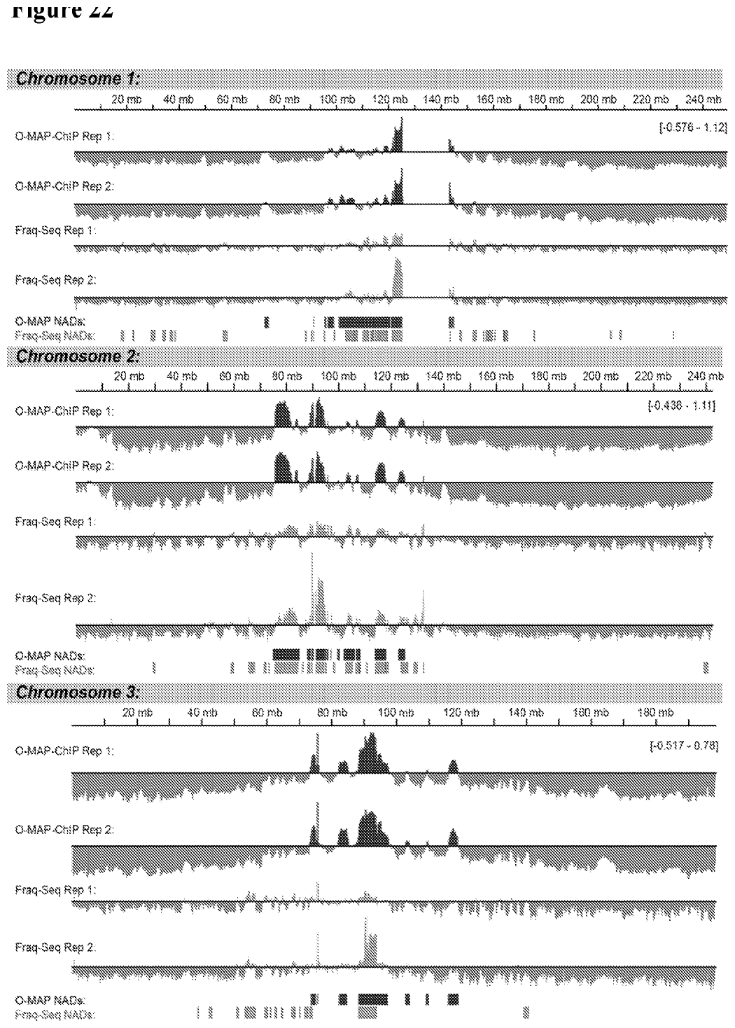

As a more challenging target, it was next sought to use O-MAP-ChIP to profile nucleolar-chromatin interactions in Hela cells, by targeting the 47S pre-rRNA. Mammalian nucleoli are surrounded by megabase-scale chromatin structures termed Nucleolar Associated Domains (NADs) which comprise nearly half of the cell's heterochromatin, and which are central to patterning epigenetic and transcriptional programs⁸¹. Although NADs have been previously characterized by isolating and sequencing intact nucleoli⁸², this demanding approach has been challenging to apply to more than a handful of human cell lines. In contrast, 47S O-MAP-ChIP enabled comprehensive, high-resolution maps of HeLa NADs from as few as 4×10⁶ cells, with approximately five days' hands-on time (FIG. 4e and FIG. 21). Remarkably, the O-MAP-ChIP data largely recapitulated the NAD architecture observed by biochemical fractionation⁸², with nearly 72% overlap between the two data sets (p=1.21×10⁻⁶², Fisher's exact), even though they were acquired from different cell lines (FIG. 4f). O-MAP-ChIP also appeared markedly less noisy than fractionation-sequencing, with higher agreement between biological replicates (FIG. 4e).

NAD architecture is almost universally remodeled in cancer, potentially driving epigenetic and transcriptional changes that facilitate oncogenesis^{11, 81, 83}. Yet, the functional impact of this dysregulation has been difficult to assess without robust methods for characterizing NAD architecture across cancer types. To demonstrate how O-MAP might facilitate this analysis, we applied 47S O-MAP-ChIP across four Pancreatic Ductal Adenocarcinoma (PDA) cell lines, systematically interrogating NAD organization across both the “classical” (ASPC1 and SUIT2 lines) and “basal” (8988T and Panc3.27 lines) PDA subtypes⁸⁴ (FIG. 4g). Although nucleolar morphology differs markedly between PDA subtypes⁸⁴, 47S O-MAP-ChIP revealed a set of invariant nucleolar-genomic interactions (160 NADs) conserved across all cell lines, suggesting a core PDA NAD architecture. These conserved domains comprise the most abundant NAD class (approximately 55% of NADs in any line), though sizeable groups of cell line-specific domains (30-52 NADs; 10-18%), or domains uniquely absent from a cell line (4-32 NADs; 1.4-11%) were also observed. NADs uniquely conserved in each PDA subtype-15 and six NADs in classical and basal, respectively (FIGS. 4g,h) was also observed. These variable domains may be particularly important for PDA, for which epigenetic regulation is thought to play a key role during oncogenesis and subtype specification⁸⁵. To examine this, ChromHMM analysis to identify cell line specific NAD epigenomic signatures was performed⁸⁶. This showed clear differences in zinc-finger/repeats (ZNF/Repeats) and heterochromatin domains between NADs of different cell types, with a profound loss of ZNF/Repeats in 8988T NADs (FIG. 4i). Any one of these data sets would represent the highest-resolution map of human NADs reported to date. Furthermore, these data—combined with the nucleolar proteomic (FIG. 2) and transcriptomic analyses (FIG. 3a-g)—demonstrated O-MAP's capacity to enable a complete, “multi-omic” dissection of an RNA-scaffolded compartment using a common experimental workflow.

### Example 6: O-MAP is Portable Across Specimen Types and Target RNAs

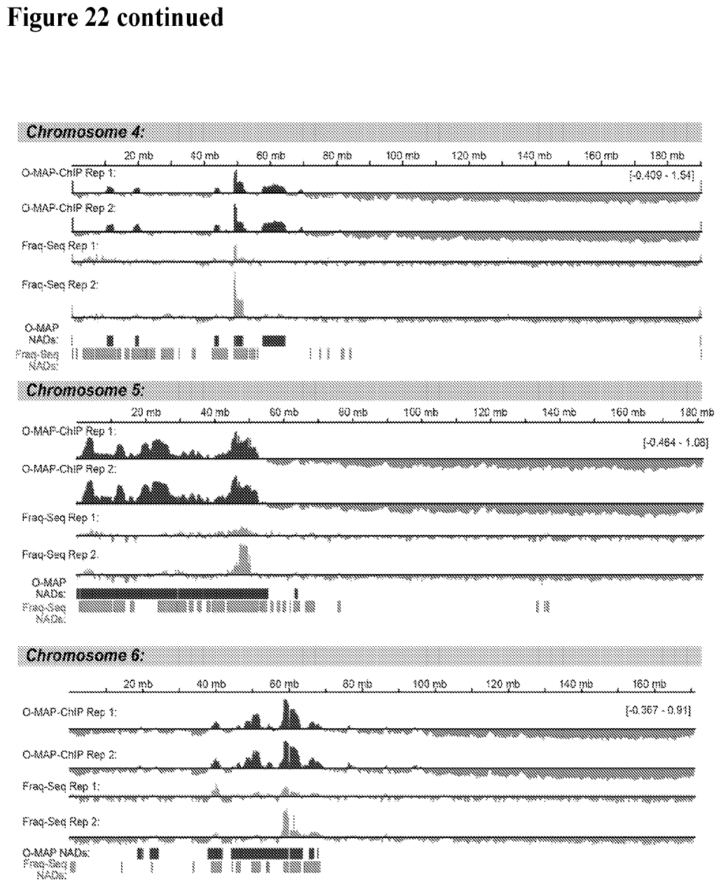

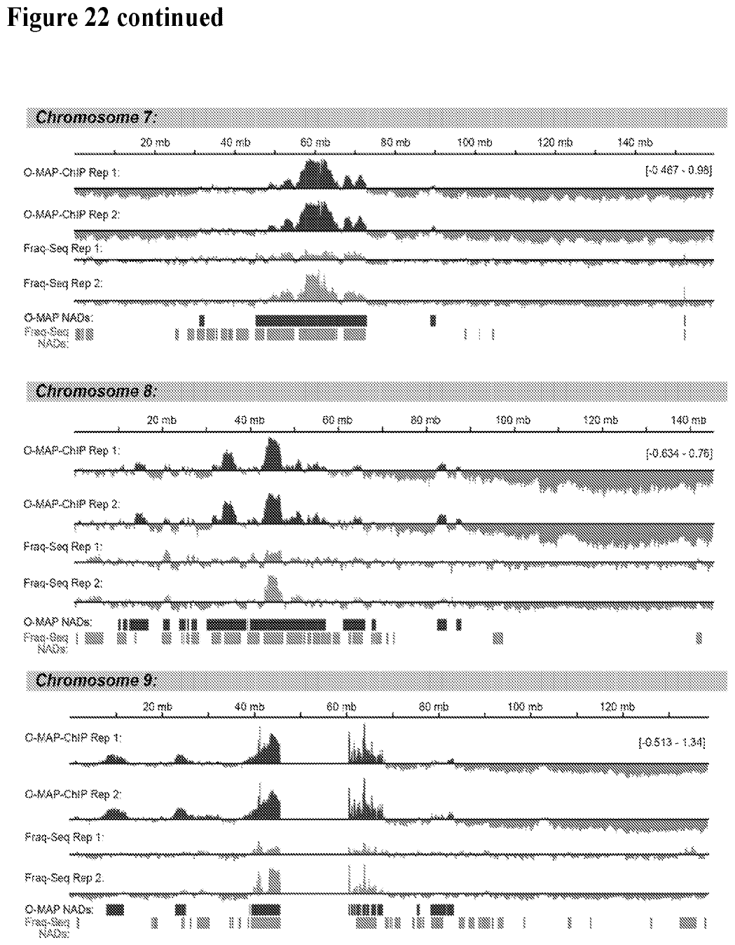

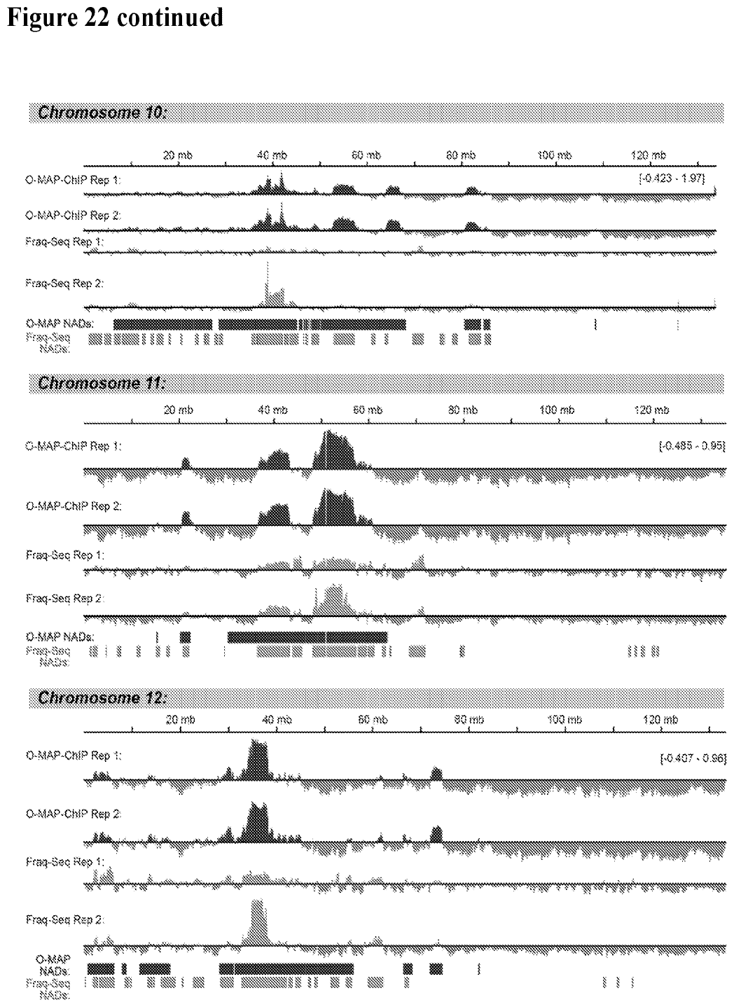

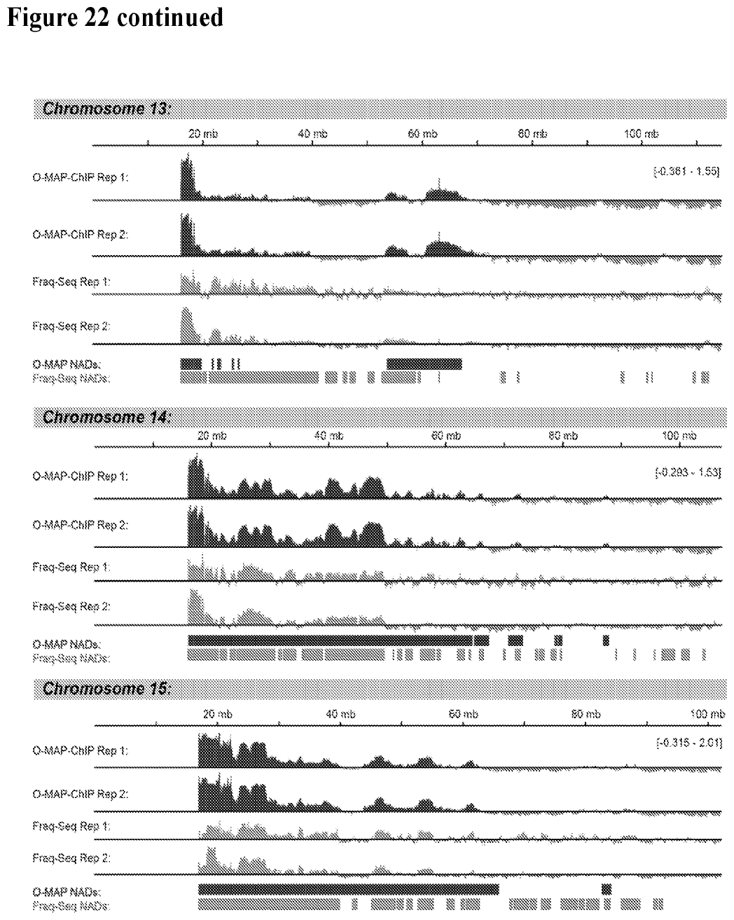

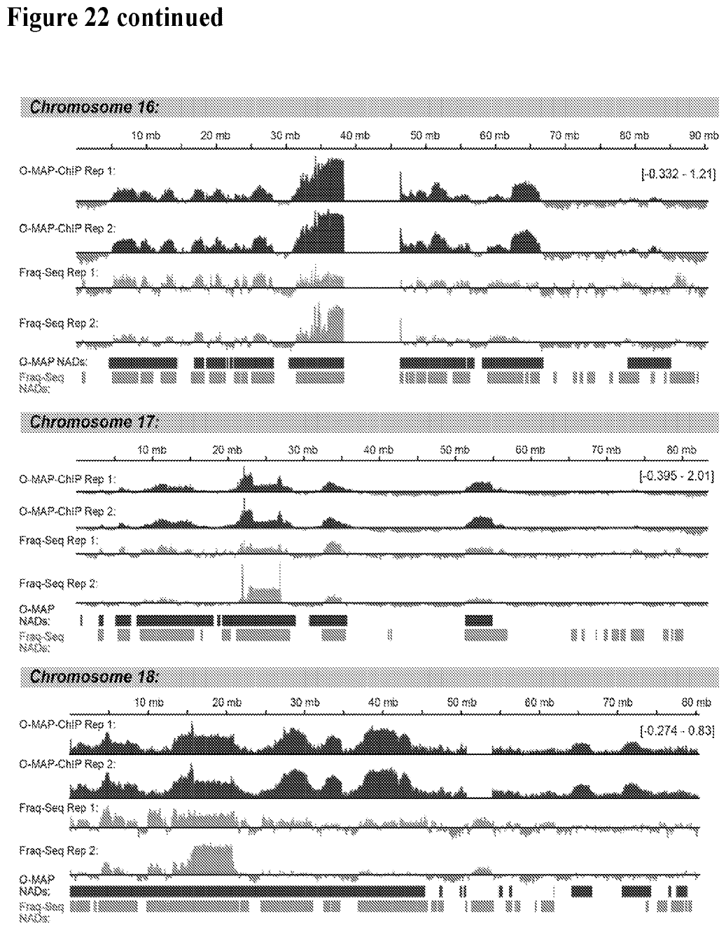

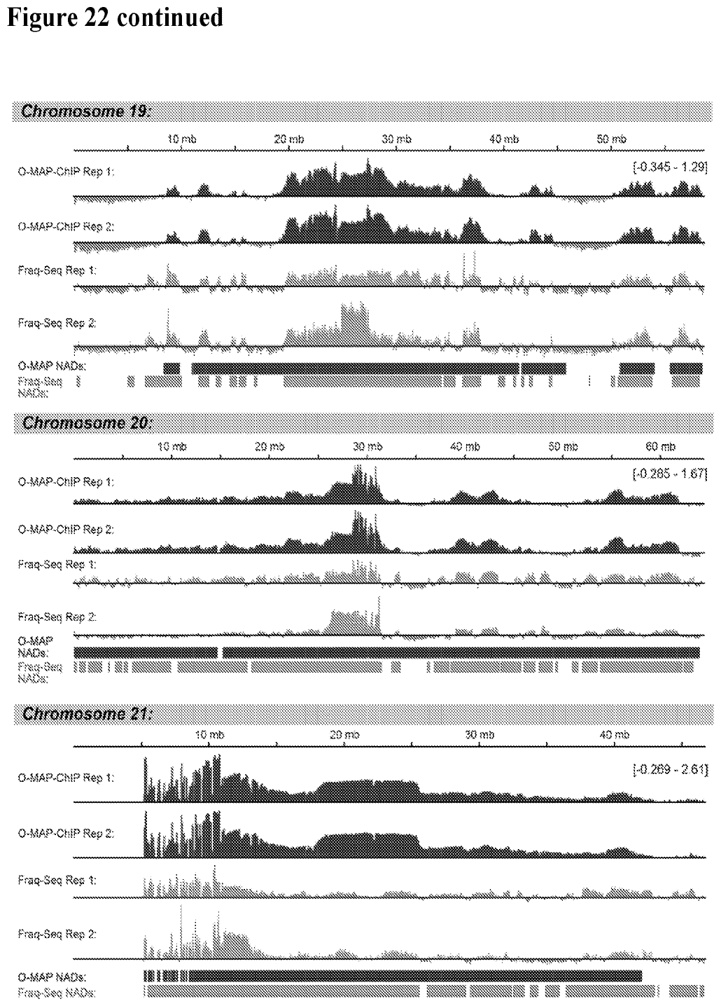

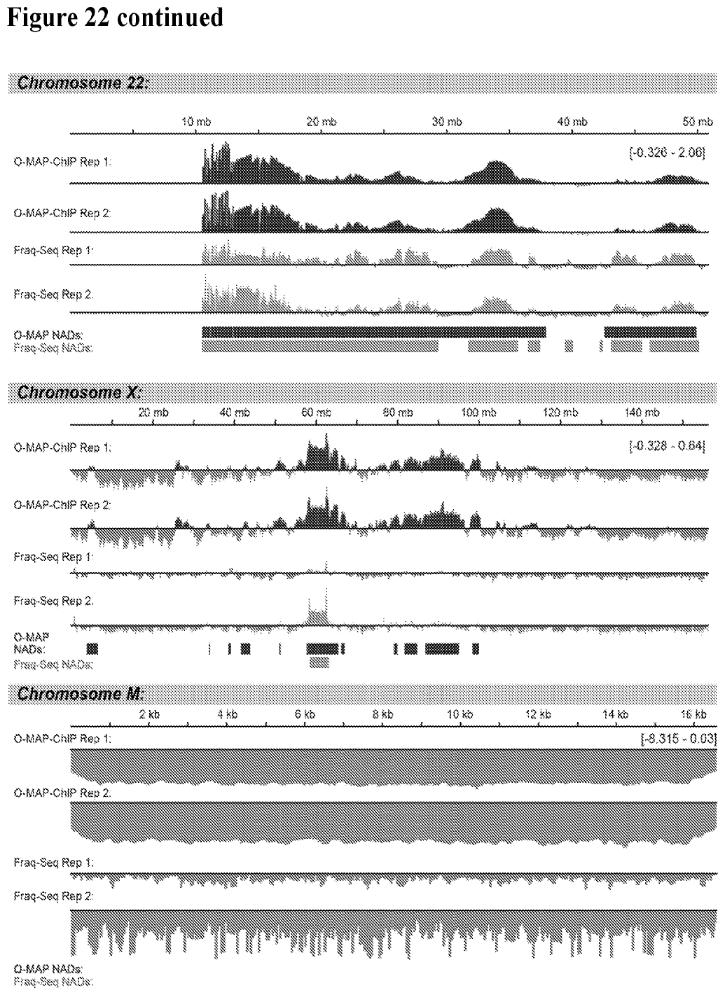

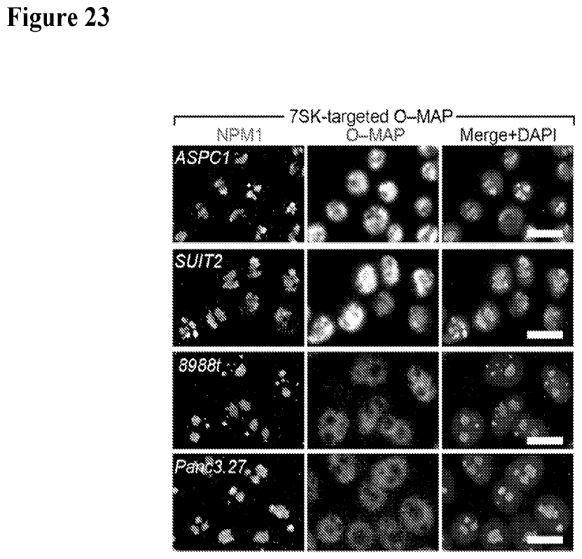

A chief limitation of genetically encoded proximity-biotinylation methods is that they require the generation of a custom-built transgenic line for each new cell culture model, tissue, or organism under interrogation²⁰. It was hypothesized that O-MAP—which doesn't require genetic manipulation to program its spatial targeting—would overcome this limitation. To test this, it was first sought to apply O-MAP to the same RNA in an array of different cultured mammalian cell lines, using the 47S-pre-rRNA as a target. In all cases, identical hybridization- and in situ-labeling conditions to those optimized in Hela cells, and analogous (species-specific) 47S-targeting probes was used (FIG. 1b). This revealed O-MAP to be remarkably modular, catalyzing precise, nucleolar-targeted biotinylation in every line tested (FIG. 5a and FIG. 22). Notably, robust labeling irrespective of nucleolar volume, density, or morphology—factors that can limit affinity-purification-and fractionation-based approaches. This modularity enabled us to perform parallelized discovery established methods was observed.

These results suggested that O-MAP might also be portable to specimens for which transgenic proximity-biotinylation approaches would be more challenging, or altogether intractable. This possibility was explored using two such specimen types: patient-derived PDA tumor organoids⁸⁷, and fixed mouse embryo tissue sections. Although each sample-type required modest re-optimization of hybridization- and in situ-labeling conditions, robust and specific 47S-targeted O-MAP in both was produced (FIG. 5b, c). This demonstrated O-MAP's portability beyond two-dimensional cell culture, and implies its potential application for RNA-interaction discovery in clinically relevant contexts⁸⁷.

Having demonstrated O-MAP's portability across biological samples, it was next sought to examine its portability to different RNAs. To this end, O-MAP to an array of transcripts with diverse lengths, expression levels, sequence composition, biogenesis pathways, and subcellular localization was targeted. For each new target, primary probes were designed using the OligoMiner pipeline³⁴, and the optimal in situ biotinylation time was determined empirically via a labeling time course. Encouragingly, in each case, O-MAP yielded prominent in situ biotinylation that recapitulated the target's known subcellular localization. Probe Validation Assay (FIG. 1d) to confirm the RNA-targeting accuracy of the probe pool was then used. Remarkably, in all cases the in situ biotinylation and RNA-FISH signals exhibited a high degree of overlap, underscoring O-MAP's precision at each target (FIG. 6a). This included highly abundant transcripts like the chromatin-regulatory IncRNAs MALAT1 and NEAT1, modestly expressed targets like the WDR7 mRNA⁸⁸, and low abundance RNAs like Firre and Kcnq1ot1. Notably, obtaining robust O-MAP at low-abundance targets required large probe pools (100-500 oligos) to bolster the number of HRP molecules recruited to the target transcript, as well as longer labeling times (60-120 minutes).

Nearly all mammalian RNAs are spliced or otherwise processed, and as a result the same transcript may contain distinct sequence elements (e.g. introns, leader segments, transcribed spacers) at different points during its biogenesis. It was hypothesized that O-MAP could be targeted to these unique elements, enabling differential analysis at various stages of an RNA's lifecycle. For example, targeting O-MAP to each of the 47S pre-rRNA “transcribed spacer” domains yielded distinct patterns of sub-nucleolar biotinylation (FIG. 9), consistent with these domains' residence in disparate cohorts of pre-rRNA species³⁸. It was reasoned that a similar strategy could be applied to nascent Pol II transcripts, by targeting O-MAP to introns. As a first test case Xist, since its nascent transcripts are thought to denote a specialized chromatin domain termed the X-chromosome Inactivation Center (XIC) that is critical for XCI initiation was selected^{45, 70} (FIG. 6b, left). To examine if O-MAP can specifically label the XIC, a variation of our probe-validation assay (FIG. 1d), in which HRP was targeted to Xist introns (nascent transcripts) and RNA-FISH was targeted to Xist exons (mature transcripts) was used. As predicted, pre-Xist O-MAP induced small, bright biotinylation puncta localized within larger “clouds” of Xist RNA-FISH, suggesting precise biotinylation of a small subcompartment within the Xi (FIG. 6b). This demonstrated that O-MAP can distinguish between populations of a target RNA's isoforms (i.e. mature vs. nascent), and further suggested a general strategy for using intron-targeted O-MAP to precisely probe subnuclear structures that would be impossible to purify biochemically.

In this disclosure, a nearly universal method for applying proximity-labeling to RNA, by co-opting the mechanics of RNA-FISH to precisely deploy biotinylating enzymes to a target transcript without genetic modification has been developed. This approach, O-MAP, enabled the building of a robust toolkit for elucidating the proteins (O-MAP-MS), transcripts (O-MAP-Seq), and genomic loci (O-MAP-ChIP) near an RNA of interest. This toolkit holds considerable advantages over established RNA interaction-discovery methods, offering superior precision, biological context, flexibility, case of use, and cost. Moreover, O-MAP's ability to probe higher-order interactions within a transcript's subcellular “neighborhood” enables unprecedented analysis of RNA-mediated compartmentalization and is a powerful new approach for spatial cell biology.

The present invention overcomes the previous technologies limitations by using a straightforward Probe Validation Assay that enabled the rapid identification and elimination of off-targeting probes (FIGS. 1d-e, and 6). Because O-MAP enzymatically amplified the number of biotin moieties per hybridized oligo probe, and enabled interacting molecules to be directly captured (rather than co-purified by pulldown), it is markedly more efficient that previous techniques. The multi-omic analysis of HeLa nucleoli obtained with O-MAP-MS, O-MAP-Seq, and O-MAP-ChIP (FIGS. 2, 3a-g, and 4e-i) collectively required fewer cells than would be needed for a single replicate of ChIRP-MS¹⁷. This higher efficiency, and the ease with which O-MAP is deployed to different specimen types (FIG. 5), enabled parallelized analysis across tissues, cell types, or other experimental variables (e.g FIG. 4g-i). Unlike previously established techniques, proximity-biotinylation approaches like O-MAP are uniquely suited to probe dynamic, higher-order interactions that are too transient or fragile to survive a pulldown.

Compared to RNA-targeted live-cell proximity-labeling methods, O-MAP is more spatially precise, more applicable across target transcripts and specimen types, and is significantly easier. Live cell approaches (e.g. RapID, MS2/Cas13-APEX, CRUIS) assemble artificial complexes between the labeling enzyme and its RNA target, by transgenically overexpressing these components fused to localization sequences or binding motifs^{21, 27, 28, 89}. Although such transgenic approaches have bad some success, they must contend with substantial background labeling catalyzed by unbound or off-targeted enzymes^{27, 28} (FIG. 1c), and with artifacts from overexpressing the target RNA outside of its native context⁹⁰. Both issues can be particularly problematic with low-abundance transcripts. In contrast, the present invention doesn't require transgenic manipulation to control enzyme localization. The sequences, abundances, and hybridization parameters of its HRP-targeting oligos were explicitly controlled to minimize off-targeting³⁵, and residual, mislocalized probes can be removed by stringent washing. This strategy enabled endogenous RNAs—even low abundance transcripts—to be biotinylated with nearly undetectable background (FIGS. 1b and 6). It furthermore enabled O-MAP to be deployed in contexts where genetic manipulation would be challenging or altogether impossible, including pre-fixed tissue samples (FIG. 5c). Applying O-MAP in samples like these, such as clinical isolates, is a powerful avenue for biomarker and therapeutic target discovery.

The methods used in the prior art are markedly noisier than O-MAP's “landing-pad” approach (FIG. 8), and their lack of modularity precludes strategies like our Probe Validation Assay (FIGS. 1d-e and 6), a vital optimization tool. Moreover, O-MAP is compatible with quantitative tandem mass-tag-based proteomics (FIG. 2), it enables ChIP-like genomic interaction discovery (FIG. 4), and it can be used with conventional formalin-fixed tissues (FIG. 5). O-MAP is also considerably cheaper, using inexpensive, chemically unmodified primary oligos, in lieu of DIG-conjugated probes. And critically, O-MAP doesn't require the synthesis of custom proteins or other reagents, using entirely off-the-shelf parts.

This disclosure illustrates O-MAP's unique ability to discover compartment-level interactions that are opaque to current methods. The present O-MAP-MS analysis represents the first proteomic characterization of this enigmatic RNA's subnuclear compartment (FIG. 2c,d). O-MAP-Seq likewise enabled a first-of-its-kind characterization of the Xist-proximal transcriptome, revealing a new XCI-escape gene and the unanticipated co-compartmentalization of Xist with other chromatin-regulatory RNAs (FIG. 3h-j). Notably, the novel interaction between Xist and Kcnq1ot1 (FIG. 3h,j) appears to occur at distances that would be intractable by currently used RNA-RNA interaction-discovery tools, as these tools predominantly query direct base-pairing contacts⁹⁴. This interaction is also paralleled in the O-MAP-ChIP analysis, which revealed a cohort of putative long-range interactions between the inactive X chromosome and autosomal loci (FIG. 4d). Trans-chromosomal contacts of this sort are notoriously challenging to probe using conventional, genomics tools⁹⁵. This demonstrates that O-MAP-ChIP enabled novel characterization of subnuclear compartments at currently unattainable distance scales, providing powerful new insight into higher-order genome organization. O-MAP-ChIP also enabled a parallelized, high-depth analysis of NAD structure across cell lines, revealing both conserved and variable elements of NAD architecture (FIG. 4e-i). Extending this analysis to other lines, and complementing it with O-MAP-MS and O-MAP-Seq, enabled an unprecedented molecular characterization of nucleolar architectural remodeling during oncogenesis⁸³.

### REFERNCES

1. Hentze, M.W., Castello, A., Schwarzl, T. & Preiss, T. A brave new world of RNA-binding proteins. Nat Rev Mol Cell Biol 19, 327-341 (2018).
2. Queiroz, R.M.L. et al. Comprehensive identification of RNA-protein interactions in any organism using orthogonal organic phase separation (OOPS). Nat Biotechnol 37, 169-178 (2019).
3. Schueler, M. et al. Differential protein occupancy profiling of the mRNA transcriptome. Genome Biol 15, R15 (2014).
4. Muller-McNicoll, M. & Neugebauer, K.M. How cells get the message: dynamic assembly and function of mRNA-protein complexes. Nat Rev Genet 14, 275-287 (2013).
5. Mitchell, S.F. & Parker, R. Principles and properties of eukaryotic mRNPs. Mol Cell 54, 547-558 (2014).
6. Bhat, P., Honson, D. & Guttman, M. Nuclear compartmentalization as a mechanism of quantitative control of gene expression. Nat Rev Mol Cell Biol 22, 653-670 (2021).
7. Roden, C. & Gladfelter, A.S. RNA contributions to the form and function of biomolecular condensates. Nat Rev Mol Cell Biol 22, 183-195 (2021).
8. Thakur, J. & Henikoff, S. Architectural RNA in chromatin organization. Biochem Soc Trans 48, 1967-1978 (2020).
9. Nickerson, J.A., Krochmalnic, G., Wan, K.M. & Penman, S. Chromatin architecture and nuclear RNA. Proc Natl Acad Sci USA 86, 177-181 (1989).
10. Nozawa, R.S. & Gilbert, N. RNA: Nuclear Glue for Folding the Genome. Trends Cell Biol 29, 201-211 (2019).
11. Farley, K.I., Surovtseva, Y., Merkel, J. & Baserga, S.J. Determinants of mammalian nucleolar architecture. Chromosoma 124, 323-331 (2015).
12. Mao, Y.S., Zhang, B. & Spector, D.L. Biogenesis and function of nuclear bodies. Trends Genet 27, 295-306 (2011).
13. Hofmann, S., Kedersha, N., Anderson, P. & Ivanov, P. Molecular mechanisms of stress granule assembly and disassembly. Biochim Biophys Acta Mol Cell Res 1868, 118876 (2021).
14. Simon, M.D. & Machyna, M. Principles and Practices of Hybridization Capture Experiments to Study Long Noncoding RNAs That Act on Chromatin. Cold Spring Harb Perspect Biol 11 (2019).
15. Ramanathan, M., Porter, D.F. & Khavari, P.A. Methods to study RNA-protein interactions. Nat Methods 16, 225-234 (2019).
16. McDonel, P. & Guttman, M. Approaches for Understanding the Mechanisms of Long Noncoding RNA Regulation of Gene Expression. Cold Spring Harb Perspect Biol 11 (2019).
17. Chu, C. & Chang, H.Y. ChIRP-MS: RNA-Directed Proteomic Discovery. Methods Mol Biol 1861, 37-45 (2018).
18. Simon, M.D. et al. The genomic binding sites of a noncoding RNA. Proc Natl Acad Sci USA 108, 20497-20502 (2011).
19. Mili, S. & Steitz, J.A. Evidence for reassociation of RNA-binding proteins after cell lysis: implications for the interpretation of immunoprecipitation analyses. RNA 10, 1692-1694 (2004).
20. Qin, W., Cho, K.F., Cavanagh, P.E. & Ting, A.Y. Deciphering molecular interactions by proximity labeling. Nat Methods 18, 133-143 (2021).
21. Ramanathan, M. et al. RNA-protein interaction detection in living cells. Nat Methods 15, 207-212 (2018).
22. Engel, K.L. et al. Analysis of subcellular transcriptomes by RNA proximity labeling with Halo-seq. Nucleic Acids Res 50, e24 (2022).
23. Kaewsapsak, P., Shechner, D.M., Mallard, W., Rinn, J.L. & Ting, A.Y. Live-cell mapping of organelle-associated RNAs via proximity biotinylation combined with protein-RNA crosslinking. Elife 6 (2017).
24. Rhee, H.W. et al. Proteomic mapping of mitochondria in living cells via spatially restricted enzymatic tagging. Science 339, 1328-1331 (2013).
25. Fazal, F.M. et al. Atlas of Subcellular RNA Localization Revealed by APEX-Seq. Cell 178, 473-490 e426 (2019).
26. Chen, Y. et al. Mapping 3D genome organization relative to nuclear compartments using TSA-Seq as a cytological ruler. J Cell Biol 217, 4025-4048 (2018).
27. Han, Y. et al. Directed Evolution of Split APEX2 Peroxidase. ACS Chem Biol 14, 619-635 (2019).
28. Han, S. et al. RNA-protein interaction mapping via MS2- or Cas13-based APEX targeting. Proc Natl Acad Sci USA 117, 22068-22079 (2020).
29. Lobingier, B.T. et al. An Approach to Spatiotemporally Resolve Protein Interaction Networks in Living Cells. Cell 169, 350-360 e312 (2017).
30. Carter, D., Chakalova, L., Osborne, C.S., Dai, Y.F. & Fraser, P. Long-range chromatin regulatory interactions in vivo. Nat Genet 32, 623-626 (2002).
31. Yap, K., Chung, T.H. & Makeyev, E.V. Hybridization-proximity labeling reveals spatially ordered interactions of nuclear RNA compartments. Mol Cell 82, 463-478 e411 (2022).
32. Raj, A., van den Bogaard, P., Rifkin, S.A., van Oudenaarden, A. & Tyagi, S. Imaging individual mRNA molecules using multiple singly labeled probes. Nat Methods 5, 877-879 (2008).
33. Raj, A. & Rinn, J.L. Illuminating Genomic Dark Matter with RNA Imaging. Cold Spring Harb Perspect Biol 11 (2019).
34. Beliveau, B.J. et al. OligoMiner provides a rapid, flexible environment for the design of genome-scale oligonucleotide in situ hybridization probes. Proc Natl Acad Sci USA 115, E2183-E2192 (2018).
35. Kishi, J.Y. et al. SABER amplifies FISH: enhanced multiplexed imaging of RNA and DNA in cells and tissues. Nat Methods 16, 533-544 (2019).
36. Pederson, T. The nucleolus. Cold Spring Harb Perspect Biol 3 (2011).
37. Padovan-Merhar, O. et al. Single mammalian cells compensate for differences in cellular volume and DNA copy number through independent global transcriptional mechanisms. Mol Cell 58, 339-352 (2015).
38. Henras, A.K., Plisson-Chastang, C., O'Donohue, M.F., Chakraborty, A. & Gleizes, P.E. An overview of pre-ribosomal RNA processing in eukaryotes. Wiley Interdiscip Rev RNA 6, 225-242 (2015).
39. Raap, A.K. et al. Ultra-sensitive FISH using peroxidase-mediated deposition of biotin- or fluorochrome tyramides. Hum Mol Genet 4, 529-534 (1995).
40. Dopie, J., Sweredoski, M.J., Moradian, A. & Belmont, A.S. Tyramide signal amplification mass spectrometry (TSA-MS) ratio identifies nuclear speckle proteins. J Cell Biol 219 (2020).
41. Beliveau, B.J. et al. Versatile design and synthesis platform for visualizing genomes with Oligopaint FISH probes. Proc Natl Acad Sci USA 109, 21301-21306 (2012).
42. Wendeberg, A. Fluorescence in situ hybridization for the identification of environmental microbes. Cold Spring Harb Protoc 2010, pdb prot5366 (2010).
43. Prasanth, K.V. et al. Nuclear organization and dynamics of 7SK RNA in regulating gene expression. Mol Biol Cell 21, 4184-4196 (2010).
44. Qin, W., Myers, S.A., Carey, D.K., Carr, S.A. & Ting, A.Y. Spatiotemporally-resolved mapping of RNA binding proteins via functional proximity labeling reveals a mitochondrial mRNA anchor promoting stress recovery. Nat Commun 12, 4980 (2021).
45. Plath, K., Mlynarczyk-Evans, S., Nusinow, D.A. & Panning, B. Xist RNA and the mechanism of X chromosome inactivation. Annu Rev Genet 36, 233-278 (2002).
46. Ahmad, Y., Boisvert, F.M., Gregor, P., Cobley, A. & Lamond, A.I. NOPdb: Nucleolar Proteome Database—2008 update. Nucleic Acids Res 37, D181-184 (2009).
47. Stenstrom, L. et al. Mapping the nucleolar proteome reveals a spatiotemporal organization related to intrinsic protein disorder. Mol Syst Biol 16, e9469 (2020).
48. Hung. V. et al. Proteomic mapping of the human mitochondrial intermembrane space in live cells via ratiometric APEX tagging. Mol Cell 55, 332-341 (2014).
49. Li, J. et al. TMTpro reagents: a set of isobaric labeling mass tags enables simultaneous proteome-wide measurements across 16 samples. Nat Methods 17, 399-404 (2020).
50. McHugh, C.A. & Guttman, M. RAP-MS: A Method to Identify Proteins that Interact Directly with a Specific RNA Molecule in Cells. Methods Mol Biol 1649, 473-488 (2018).
51. Andersen, J.S. et al. Directed proteomic analysis of the human nucleolus. Curr Biol 12, 1-11 (2002).
52. Diribame, G. & Bensaude, O. 7SK RNA, a non-coding RNA regulating P-TEFb, a general transcription factor. RNA Biol 6, 122-128 (2009).
53. Flynn, R.A. et al. 7SK-BAF axis controls pervasive transcription at enhancers. Nat Struct Mol Biol 23, 231-238 (2016).
54. Uhlen, M. et al. Towards a knowledge-based Human Protein Atlas. Nat Biotechnol 28, 1248-1250 (2010).
55. Zhou, Y. et al. Metascape provides a biologist-oriented resource for the analysis of systems-level datasets. Nat Commun 10, 1523 (2019).
56. Subramanian, A. et al. Gene set enrichment analysis: a knowledge-based approach for interpreting genome-wide expression profiles. Proc Natl Acad Sci USA 102, 15545-15550 (2005).
57. Spector, D.L. & Lamond, A.I. Nuclear speckles, Cold Spring Harb Perspect Biol 3 (2011).
58. UniProt, C. UniProt: the universal protein knowledgebase in 2021. Nucleic Acids Res 49, D480-D489 (2021).
59. Jan, C.H., Williams, C.C. & Weissman, J.S. Principles of ER cotranslational translocation revealed by proximity-specific ribosome profiling. Science 346, 1257521 (2014).
60. Engreitz, J.M. et al. RNA-RNA interactions enable specific targeting of noncoding RNAs to nascent Pre-mRNAs and chromatin sites. Cell 159, 188-199 (2014).
61. Pederson, T. & Politz, J.C. The nucleolus and the four ribonucleoproteins of translation. J Cell Biol 148, 1091-1095 (2000).
62. Bachellerie, J.P., Cavaille, J. & Huttenhofer, A. The expanding snoRNA world. Biochimie 84, 775-790 (2002).
63. Caudron-Herger, M., Pankert, T. & Rippe, K. Regulation of nucleolus assembly by non-coding RNA polymerase II transcripts. Nucleus 7, 308-318 (2016).
64. Bai, B., Liu, H. & Laiho, M. Small RNA expression and deep sequencing analyses of the nucleolus reveal the presence of nucleolus-associated microRNAs. FEBS Open Bio 4, 441-449 (2014).
65. Adiconis, X. et al. Comparative analysis of RNA sequencing methods for degraded or low-input samples. Nat Methods 10, 623-629 (2013).
66. Wu, M. et al. IncRNA SLERT controls phase separation of FC/DFCs to facilitate Pol I transcription. Science 373, 547-555 (2021).
67. Barutcu, A.R. et al. Systematic mapping of nuclear domain-associated transcripts reveals speckles and lamina as hubs of functionally distinct retained introns. Mol Cell 82, 1035-1052 e1039 (2022).
68. Caudron-Herger, M. et al. Alu element-containing RNAs maintain nucleolar structure and function. EMBO J 34, 2758-2774 (2015).
69. Disteche, C.M. Dosage compensation of the sex chromosomes. Annu Rev Genet 46, 537-560 (2012).
70. Loda, A. & Heard, E. Xist RNA in action: Past, present, and future. PLoS Genet 15, e1008333 (2019).
71. Disteche, C.M. & Berletch, J.B. X-chromosome inactivation and escape. J Genet 94, 591-599 (2015).
72. Brockdorff, N. Localized accumulation of Xist RNA in X chromosome inactivation. Open Biol 9, 190213 (2019).
73. Strehle, M. & Guttman, M. Xist drives spatial compartmentalization of DNA and protein to orchestrate initiation and maintenance of X inactivation. Curr Opin Cell Biol 64, 139-147 (2020).
74. Lingenfelter, P.A. et al. Escape from X inactivation of Smex is preceded by silencing during mouse development. Nat Genet 18, 212-213 (1998).
75. Covarrubias, S. et al. CRISPR/Cas-based screening of long non-coding RNAs (IncRNAs) in macrophages with an NF-kappaB reporter. J Biol Chem 292, 20911-20920 (2017).
76. Lyon, M.F. X-chromosome inactivation: a repeat hypothesis. Cytogenet Cell Genet 80, 133-137 (1998).
77. Pandey, R.R. et al. Kcnq1ot1 antisense noncoding RNA mediates lineage-specific transcriptional silencing through chromatin-level regulation. Mol Cell 32, 232-246 (2008).
78. Simon, M.D. et al. High-resolution Xist binding maps reveal two-step spreading during X-chromosome inactivation. Nature 504, 465-469 (2013).
79. Engreitz, J.M. et al. The Xist IncRNA exploits three-dimensional genome architecture to spread across the X chromosome. Science 341, 1237973 (2013).
80. Jachowicz, J.W. et al. Xist spatially amplifies SHARP/SPEN recruitment to balance chromosome-wide silencing and specificity to the X chromosome. Nat Struct Mol Biol 29, 239-249 (2022).
81. Matheson, T.D. & Kaufman, P.D. Grabbing the genome by the NADs. Chromosoma 125, 361-371 (2016).
82. van Koningsbruggen, S. et al. High-resolution whole-genome sequencing reveals that specific chromatin domains from most human chromosomes associate with nucleoli. Mol Biol Cell 21, 3735-3748 (2010).
83. Derenzini, M. et al. Nucleolar function and size in cancer cells. Am J Pathol 152, 1291-1297 (1998).
84. Diwakarla, C., Hannan, K., Hein, N. & Yip, D. Advanced pancreatic ductal adenocarcinoma—Complexities of treatment and emerging therapeutic options. World J Gastroenterol 23, 2276-2285 (2017).
85. Lomberk, G. et al. Distinct epigenetic landscapes underlie the pathobiology of pancreatic cancer subtypes. Nat Commun 9, 1978 (2018).
86. Ernst, J. & Kellis, M. ChromHMM: automating chromatin-state discovery and characterization. Nat Methods 9, 215-216 (2012).
87. Boj, S.F. et al. Organoid models of human and mouse ductal pancreatic cancer. Cell 160, 324-338 (2015).
88. Course, M.M. et al. Evolution of a Human-Specific Tandem Repeat Associated with ALS. Am J Hum Genet 107, 445-460 (2020).
89. Zhang, Z. et al. Capturing RNA-protein interaction via CRUIS. Nucleic Acids Res 48, e52 (2020).
90. Bassett, A.R. et al. Considerations when investigating IncRNA function in vivo. Elife 3, e03058 (2014).
91. Jiang, S. et al. A proteomics approach to the cell-surface interactome using the enzyme-mediated activation of radical sources reaction. Proteomics 12, 54-62 (2012).
92. Rees, J.S., Li, X.W., Perrett, S., Lilley, K.S. & Jackson, A.P. Selective Proteomic Proximity Labeling Assay Using Tyramide (SPPLAT): A Quantitative Method for the Proteomic Analysis of Localized Membrane-Bound Protein Clusters. Curr Protoc Protein Sci 80, 19 27 11-19 27 18 (2015).
93. Bar, D.Z. et al. Biotinylation by antibody recognition-a method for proximity labeling. Nat Methods 15, 127-133 (2018).
94. Kudla, G., Wan, Y. & Helwak, A. RNA Conformation Capture by Proximity Ligation. Annu Rev Genomics Hum Genet 21, 81-100 (2020).
95. Dekker, J. & Misteli, T. Long-Range Chromatin Interactions. Cold Spring Harb Perspect Biol 7, a019356 (2015).
96. Schnell, U., Dijk, F., Sjollema, K.A. & Giepmans, B.N. Immunolabeling artifacts and the need for live-cell imaging. Nat Methods 9, 152-158 (2012).
97. Cech, T.R. & Steitz, J.A. The noncoding RNA revolution-trashing old rules to forge new ones. Cell 157, 77-94 (2014).
98. Jain, A. & Vale, R.D. RNA phase transitions in repeat expansion disorders. Nature 546, 243-247 (2017).
99. Shechner, D.M. & Bartel, D.P. The structural basis of RNA-catalyzed RNA polymerization. Nat Struct Mol Biol 18, 1036-1042 (2011).
100. Rappsilber, J., Ishihama, Y. & Mann, M. Stop and go extraction tips for matrix-assisted laser desorption/ionization, nanoelectrospray, and LC/MS sample pretreatment in proteomics. Anal Chem 75, 663-670 (2003).
101. Eng, J.K., Jahan, T.A. & Hoopmann, M.R. Comet: an open-source MS/MS sequence database search tool. Proteomics 13, 22-24 (2013).
102. Rad, R. et al. Improved Monoisotopic Mass Estimation for Deeper Proteome Coverage. J Proteome Res 20, 591-598 (2021).
103. Schweppe, D.K. et al. Full-Featured, Real-Time Database Searching Platform Enables Fast and Accurate Multiplexed Quantitative Proteomics. J Proteome Res 19, 2026-2034 (2020).
104. Kim, D., Paggi, J.M., Park, C., Bennett, C. & Salzberg, S.L. Graph-based genome alignment and genotyping with HISAT2 and HISAT-genotype. Nat Biotechnol 37, 907-915 (2019).
105. Mars, J.C., Sabourin-Felix, M., Tremblay, M.G. & Moss, T. A Deconvolution Protocol for ChIP-Seq Reveals Analogous Enhancer Structures on the Mouse and Human Ribosomal RNA Genes. G3 (Bethesda) 8, 303-314 (2018).
106. Danecek, P. et al. Twelve years of SAMtools and BCFtools. Gigascience 10 (2021).
107. Ramirez, F., Dundar, F., Diehl, S., Gruning, B.A. & Manke, T. deepTools: a flexible platform for exploring deep-sequencing data. Nucleic Acids Res 42, W187-191 (2014).
108. Shumate, A., Wong, B., Pertea, G. & Pertea, M. Improved transcriptome assembly using a hybrid of long and short reads with String Tie. PLoS Comput Biol 18, e1009730 (2022).
109. Love, M.I., Huber, W. & Anders, S. Moderated estimation of fold change and dispersion for RNA-seq data with DESeq2. Genome Biol 15, 550 (2014).
110. Jin, Y., Tam, O.H., Paniagua, E. & Hammell, M. TEtranscripts: a package for including transposable elements in differential expression analysis of RNA-seq datasets. Bioinformatics 31, 3593-3599 (2015).
111. Dobin, A. et al. STAR: ultrafast universal RNA-seq aligner. Bioinformatics 29, 15-21 (2013).
112. Waskom, M.L. seaborn: statistical data visualization. Journal of Open Source Software 6 (2021).
113. Hunter, J.D. Matplotlib: A 2D graphics environment. Computing in Science & Engineering 9, 90-95 (2007).
114. Langdon, W.B. Performance of genetic programming optimised Bowtie2 on genome comparison and analytic testing (GCAT) benchmarks. BioData Min 8, 1 (2015).
115. Lund, E., Oldenburg, A.R. & Collas, P. Enriched domain detector: a program for detection of wide genomic enrichment domains robust against local variations. Nucleic Acids Res 42, e92 (2014).
116. Stovner, E.B. & Saetrom, P. epic2 efficiently finds diffuse domains in ChIP-seq data. Bioinformatics 35, 4392-4393 (2019).
117. Quinlan, A.R. & Hall, I.M. BEDTools: a flexible suite of utilities for comparing genomic features. Bioinformatics 26, 841-842 (2010).
118. Zhang, Y. et al. Model-based analysis of ChIP-Seq (MACS). Genome Biol 9, R137 (2008).
119. Bonora, G. et al. Orientation-dependent Dxz4 contacts shape the 3D structure of the inactive X chromosome. Nat Commun 9, 1445 (2018).
120. Schindelin, J. et al. Fiji: an open-source platform for biological-image analysis. Nat Methods 9, 676-682 (2012).
121. Rueden, C.T. et al. ImageJ2: ImageJ for the next generation of scientific image data. BMC Bioinformatics 18, 529 (2017).
122. Gilles, J.F., Dos Santos, M., Boudier, T., Bolte, S. & Heck, N. DiAna, an ImageJ tool for object-based 3D co-localization and distance analysis. Methods 115, 55-64 (2017).

1. A method for detecting one or more proteins, RNAs or genomic loci associated with a biomolecule of interest comprising:
a. contacting a biological sample with a first probe to produce a modified sample, wherein the first probe comprises
(i) a first binding site capable of binding the biomolecule of interest, and
(ii) one or a plurality of second binding sites each capable of binding with a detector oligonucleotide,
wherein the modified sample comprises the first binding site of the first probe bound to the biomolecule of interest;
b. contacting the modified sample with one or more detector oligonucleotides to produce a complexed sample, wherein each detector oligonucleotide comprises
(i) a first binding site capable of binding to one or more of the second binding sites of the first probe, and
(ii) an enzyme,
wherein the complexed sample comprises
(A) the first binding site of the first probe bound to the biomolecule of interest; and
(B) the first binding site of one or more of the detector oligonucleotides bound with a second binding site of the first probe one or plurality of second binding sites;
c. contacting the complexed sample with a substrate capable of being converted to a reactive visible form to produce a labeled sample, wherein the labeled sample comprises
(i) the first binding site of the first probe bound to the biomolecule of interest,
(ii) the first binding site of one or more of the detector oligonucleotides bound to a second binding site of the first probe one or plurality of second binding sites, and
a covalent linkage between the substrate and the one or more proteins associated with the biomolecule of interest; and
d. detecting the one or more proteins, RNAs or genomic loci which are associated with the biomolecule of interest.

2-3. (canceled)

4. The method of claim 1 wherein the biological sample is a fixed sample.

5. The method of claim 1 wherein the biological sample is a mammalian cell sample.

6. The method of claim 1 wherein the first probe comprises an oligonucleotide, an antibody, an aptamer, a lectin, and/or a probe conjugated to an oligonucleotide.

7. (canceled)

8. The method of claim 1 wherein the first probe comprises an oligonucleotide probe, wherein the one or a plurality of second binding sites on the first probe are each capable of base pairing with the detector oligonucleotide.

9. The method of claim 1 wherein the plurality of second binding sites comprises 2, 3, 4 or, 5 binding sites.

10. The method of claim 1 wherein the enzyme comprises horseradish peroxidase, ascorbate peroxidase, or microbial transglutaminase.

11. The method of claim 1 wherein the substrate is a tyramide compound, DAB (3,3-diamienzidine) or biotin aniline, or salts thereof.

12. (canceled)

13. The method of claim 1, comprising visualizing the one or more proteins or the biomolecule of interest.

14. The method of claim 1, wherein the method further comprises:
e. lysing the labeled sample to produce a lysed labeled sample; and
f. detecting the one or more proteins, RNAs or genomic loci which are associated with the biomolecule of interest from the lysed labeled sample.

15. A method for detecting one or more proteins associated with an RNA molecule or a genomic locus of interest comprising:
a. contacting a biological sample with a first oligonucleotide to produce a modified sample, wherein the first oligonucleotide comprises
(i) a first binding site capable of binding the RNA molecule or a genomic locus of interest, and
(ii) one or a plurality of second binding sites each capable of base pairing with a second oligonucleotide,
wherein the modified sample comprises the first binding site of the first oligonucleotide base paired with the RNA molecule or a genomic locus of interest;
b. contacting the modified sample with one or more second oligonucleotide to produce a complexed sample, wherein each second oligonucleotide comprises
(i) a first binding site capable of base pairing to the second binding site of the first oligonucleotide, and
(ii) horseradish peroxidase (HRP), wherein the complexed sample comprises
(A) the first binding site of the first oligonucleotide base paired with the RNA molecule or a genomic locus of interest; and
(B) the first binding site of one or more of the second oligonucleotide base paired with a second binding site of the first oligonucleotide one or plurality of second binding sites;
c. contacting the complexed sample with biotin tyramide and H₂O₂ to produce a biotinylated sample, wherein the biotinylated sample comprises
(i) the first binding site of the first oligonucleotide base paired with the RNA molecule or a genomic locus of interest,
(ii) the first binding site of one or more of the second oligonucleotide base paired with a second binding site of the first oligonucleotide one or plurality of second binding sites, and
a covalent linkage between the biotin tyramide and the one or more proteins associated with the RNA molecule or a genomic locus of interest; and
d. detecting the one or more proteins which are associated with to the RNA molecule or a genomic loci of interest.

16-19. (canceled)

20. The method of claim 6, wherein the detector oligonucleotide comprise DNA, RNA, PNA, LNA, or morpholino.

21-23. (canceled)

24. The method of claim 11, wherein the tyramide compound selected from the group consisting of biotin tyramide, cyanine tyramide, alkyne tyramide, and a fluorescent tyramide, or salts thereof.

25-26. (canceled)

27. The method of claim 1, wherein
a. the first probe comprises a plurality of first probes, wherein
(i) the first binding site of each of the plurality of first probes binds to a different biomolecule of interest, and
(ii) each second binding site of the first probe is capable of binding to a different detector oligonucleotide;
b. the one or more detector oligonucleotides comprises a plurality of detector oligonucleotides, wherein the first binding site of each of the plurality of detector oligonucleotides is capable of binding to the second binding site of a different first probe;
c. contacting the biological sample with the first probe comprises contacting the biological sample with the plurality of first probes to produce the modified biological sample;
d. contacting the modified biological sample with the one or more detector oligonucleotides comprises contacting the modified biological sample is with the plurality of detector oligonucleotides serially comprising,
(i) contacting the biological sample with one of the plurality of detector oligonucleotides to produce the complexed sample,
(ii) contacting the biological sample with one more visualizing agents, wherein the one or more visualizing agents are activated by the enzyme,
(iii) visualizing the one of the plurality of detector oligonucleotides with microscopy,
(iv) stripping the one of the plurality of detector oligonucleotides from the first probe, and
(v) repeating steps (i)-(iv) with a different one of the plurality of detector oligonucleotides.

28. The method of claim 27 wherein the contacting the biological sample with the plurality of first probes comprises contacting the biological sample serially or simultaneously to the plurality of first probes.

29. A kit comprising:
a. a first probe comprising
(i) a first binding site that binds to biomolecule of interest, and
(ii) a plurality of second binding sites, each second binding site capable of binding to a detector oligonucleotide; and
b. a detector oligonucleotide comprising
(i) a first binding site capable of binding to the second binding site of the first probe, and
(ii) an enzyme.

30. The kit of claim 29, wherein the first probe comprises an oligonucleotide, an antibody, an aptamer, lectin, or a probe conjugated to an oligonucleotide.

31-32. (canceled)

33. The kit of claim 29, wherein the enzyme comprises horseradish peroxidase (HRP).

34. The kit of claim 29, wherein the biomolecule of interest comprises RNA, DNA, or a protein
